(19) United States
(12) Patent Application Publication
(10) Pub. No.: US 2022/0305496 A1
(43) Pub. Date: Sep. 29, 2022

(54) METHODS AND COMPOSITIONS FOR DETECTING TARGET NUCLEIC ACIDS AND RESOLVING SAMPLE MATRICES

(71) Applicant: Detect, Inc., Guilford, CT (US)

(72) Inventors: Spencer Glantz, West Hartford, CT (US); Henry Kemble, Paris (FR); William A. Hansen, Clinton, CT (US); Sarai Meyer, Guilford, CT (US); Andrew Le, Branford, CT (US); Eleanor Rose Henn, Orange, CT (US); Molly Grun, New Haven, CT (US); Maya Overton, New Haven, CT (US); Rabib Shahab Chaudhury, Nanuet, NY (US)

(21) Appl. No.: 17/696,452

(22) Filed: Mar. 16, 2022

Related U.S. Application Data
(60) Provisional application No. 63/161,856, filed on Mar. 16, 2021.

(51) Int. Cl. B01L 7/00; C12Q 1/6806; C12N 15/10; C12Q 1/6848; B01L 3/00

(52) U.S. Cl. CPC B01L 7/52; C12Q 1/6806; C12N 15/1003; C12Q 1/6848; B01L 3/502715; B01L 2200/16; B01L 2300/0825

(57) ABSTRACT

Aspects of the disclosure relate to devices and methods for amplifying and/or detecting one or more target nucleic acid sequences (e.g., a nucleic acid sequence of one or more pathogens) in a biological sample obtained from a subject, wherein the biological sample is combined with a diluent and/or matrix resolving agent.

### RELATED APPLICATION

This application claims the benefit under 35 U.S.C. 119(e) of U.S. provisional application No. 63/161,856 filed Mar. 16, 2021, which is incorporated by reference herein in its entirety.

### FIELD

The disclosure generally relates to diagnostic devices, systems, and methods for detecting the presence of a target nucleic acid sequence.

### BACKGROUND

The ability to rapidly diagnose diseases—particularly highly infectious diseases—is critical to preserving human health. As one example, the high level of contagiousness, the high mortality rate, and the lack of a treatment for the coronavirus disease 2019 (COVID-19) have resulted in a pandemic that has already killed millions of people. The existence of rapid, accurate COVID-19 diagnostic tests could allow infected individuals to be quickly identified and isolated, which could assist with containment of the disease. In the absence of such diagnostic tests, COVID-19 may continue to spread unchecked throughout communities.

### SUMMARY

Aspects of the disclosure relate to compositions and methods for amplifying and/or detecting target analytes (e.g., nucleic acids) in a sample. The disclosure is based, in part, on methods and compositions that combine a biological sample (e.g., a subject sample) with a diluent or matrix resolving agent. In some embodiments, the diluent comprises a matrix resolving agent. In some embodiments, the biological sample comprises a mucous matrix, e.g., a nasal matrix. In some embodiments, the mucous matrix comprises an interfering enzyme. Without wishing to be bound by theory, the presence and/or structural integrity of a mucous matrix in a biological sample is thought to be an obstacle for rapid, accurate detection of target analytes (e.g., nucleic acids) in the sample. A method or composition combining the biological sample with a matrix resolving agent or diluent, e.g., comprising a matrix resolving agent, is thought to improve detection of the target analyte (e.g., relative to an otherwise similar method not combining the sample with a matrix resolving agent or diluent (e.g., comprising a matrix resolving agent)).

In some aspects, the disclosure is directed to a method for detecting a target nucleic acid, comprising combining a biological sample with a diluent to produce a sample-containing fluid and applying the sample-containing fluid to a rapid testing system or a rapid test for a nucleic acid (e.g. the target nucleic acid). In some aspects, the disclosure is directed to a method for detecting a target nucleic acid, comprising combining a biological sample with a transfer fluid and a matrix resolving agent to produce a sample-containing fluid and applying the sample-containing fluid to a rapid testing system or a rapid test for a nucleic acid (e.g. the target nucleic acid). In some embodiments, the diluent comprises a matrix-resolving agent. In some embodiments, the rapid test system or rapid test comprises a nucleic acid detection device. In some embodiments, applying the sample-containing fluid to a rapid testing device or rapid test for a nucleic acid comprises contacting a nucleic acid detection device (e.g., a vessel for a sample-containing fluid or amplification mixture in said device) with the sample-containing fluid and using the nucleic acid detection device to detect the target nucleic acid. In some embodiments, the rapid test device or rapid test comprises a lateral flow assay strip. In some embodiments, applying the sample-containing fluid to a rapid testing device or rapid test for a nucleic acid comprises contacting a lateral flow assay strip having a first end and a second end with the sample-containing fluid. In some embodiments, the lateral flow assay strip comprises an absorbent substrate having a first end and a second end; and an indicator region arranged on the substrate and configured to indicate the presence of the target nucleic acid. In some embodiments, the method comprises allowing the sample-containing fluid to move from the first end of the lateral flow assay strip to the indicator region.

In some aspects, the disclosure is directed to a rapid test system that detects a target nucleic acid, comprising a housing, a diluent comprising at least one lysis reagent, at least one amplification reagent, and a nucleic acid detection device (e.g., accommodated in the housing and arranged to receive the sample-containing fluid). In some aspects, the disclosure is directed to a rapid test system that detects a target nucleic acid, comprising a housing, a diluent comprising at least one lysis reagent, at least one amplification reagent, and a lateral flow assay strip accommodated in the housing and arranged to receive an amplified sample. In some aspects, the disclosure is directed to a rapid test system that detects a target nucleic acid, comprising a housing, at least one matrix resolving agent, at least one lysis reagent, at least one amplification reagent, and a nucleic acid detection device (e.g., accommodated in the housing and arranged to receive the sample-containing fluid). In some aspects, the disclosure is directed to a rapid test system that detects a target nucleic acid, comprising a housing, at least one matrix resolving agent, at least one lysis reagent, at least one amplification reagent, and a lateral flow assay strip accommodated in the housing and arranged to receive an amplified sample. In some embodiments, the diluent comprises at least one matrix resolving agent. In some embodiments, the lateral flow assay strip comprises an absorbent substrate having a first end and a second end, and an indicator region arranged on the substrate and configured to indicate the presence or absence of the target nucleic acid by interaction with the amplified sample.

In some aspects, the disclosure is directed to a sample preparation mixture comprising one or more matrix resolving agents and one or more lysis reagents. In some embodiments, the sample preparation mixture further comprises a diluent. In some aspects, the disclosure is directed to a method of making a sample preparation mixture comprising combining one or more matrix resolving agents and one or more lysis reagents. In some embodiments, the method of making the sample preparation mixture further comprises combining a diluent with the one or more matrix resolving agents and one or more lysis reagents. In some embodiments, the sample preparation mixture further comprises a biological sample. In some embodiments, the method of making the sample preparation mixture further comprises combining one, two, or all of one or more matrix resolving agents, one or more lysis reagents, or diluent with a biological sample.

In some embodiments, a diluent for use herein comprises at least one matrix resolving agent. In some embodiments, a diluent for use herein does not comprise a matrix resolving agent. In some embodiments, the at least one matrix resolving agent is present in a method or system described herein separate from or in addition to a diluent.

In some embodiments, a biological sample comprises a target nucleic acid. In some embodiments, a biological sample does not comprise a target nucleic acid. In some embodiments, interactions of the amplified sample with the lateral flow assay strip enable determination of a presence or an absence of the target nucleic acid.

In some embodiments, the biological sample comprises a mucous matrix. In some embodiments, the mucous matrix is a nasal matrix. In some embodiments, the biological sample comprises a nasal secretion. In some embodiments, the biological sample is an anterior nares specimen. In some embodiments, the mucous matrix is an oral matrix, a pharyngeal matrix, an esophageal matrix, or an aural matrix.

In some embodiments, the at least one matrix resolving agent comprises a reducing agent. In some embodiments, the reducing agent is selected from the group consisting of DTT (dithiothreitol), glutathione, DTE (dithioerythritol), TCEP, 2-mercaptoethanol, and any combination thereof. In some embodiments, the reducing agent is DTT. In some embodiments, the at least one matrix resolving agent comprises a mucolytic agent. In some embodiments, the mucolytic agent is N-acetyl-L-cysteine. In some embodiments, the at least one matrix resolving agent comprises a protein. In some embodiments, the protein comprises an enzyme. In some embodiments, the enzyme comprises a metalloproteinase, a disintegrin and metalloproteinase with thromospondin motifs (ADAMTS) family protein, or a functional fragment or variant thereof. In some embodiments, the metalloproteinase is a matrix metalloproteinase. In some embodiments, the at least one matrix resolving agent is in aqueous form. In some embodiments, the one or more enzymes comprises an RNase inhibitor. In some embodiments, the one or more enzymes comprises a protease. In some embodiments, the at least one matrix resolving agent comprises a chelator. In some embodiments, the chelator comprises EGTA.

In some embodiments, the diluent further comprises one or more lysis reagents. In some embodiments, a method described herein further comprises combining the biological sample or sample-containing fluid with one or more lysis reagents. In some embodiments, the one or more lysis reagents comprise an enzyme. In some embodiments, the enzyme comprises lysozyme, lysostaphin, zymolase, cellulase, protease, glycanase, or any combination thereof. In some embodiments, the one or more lysis reagents comprise a detergent. In some embodiments, the detergent comprises sodium dodecyl sulphate (SDS), Tween, 3-[(3-cholamidopropyl)dimethylammonio]-1-propanesulfonate (CHAPS), 3-[(3-cholamidopropyl)dimethylammonio]-2-hydroxy-1-propanesulfonate (CHAPSO), Triton X-100, and/or NP-40. In some embodiments, the Tween comprises Tween 20 and/or Tween 80. In some embodiments, the one or more lysis reagents are in aqueous form. In some embodiments, the one or more lysis reagents is comprised of a lysis buffer fluid.

In some embodiments, a method for detecting a target nucleic acid comprises applying heat to a biological sample prior to addition of diluent to the biological sample. In some embodiments, a method for detecting a target nucleic acid comprises applying heat to the biological sample prior to addition of a matrix resolving agent to the biological sample. In some embodiments, a method for detecting a target nucleic acid comprises applying heat to the sample-containing fluid prior to amplifying the sample. In some embodiments, a method for detecting a target nucleic acid comprises applying heat to the sample-containing fluid prior to lysing a cell of the sample. In some embodiments, a method for detecting a target nucleic acid comprises applying heat to the sample-containing fluid prior to adding a reagent to the sample (e.g., a matrix resolving agent, a lysis reagent, and/or an amplification reagent).

In some embodiments, the biological sample comprises a mucous matrix. In some embodiments, combining the biological sample with the diluent or matrix resolving agent results in a decrease in viscosity of the mucous matrix or sample-containing fluid; a decrease in viscoelasticity of the mucous matrix; a decrease in rigidity of the mucous matrix; an increase in porosity of the mucous matrix; a decrease in insolubility of the mucous matrix; an alteration in topography of the mucous matrix; and/or cleavage or digestion of a protein, glycan, or proteoglycan component of the mucous matrix. In some embodiments, combining the biological sample with the diluent or matrix resolving agent results in at least a 1, 2, 3, 4, 5, 6, 7, 8, 9, 10, 20, 30, 40, 50, 60, 70, 80, 90, 95, 99, or 100% decrease in viscosity of the mucous matrix or sample-containing fluid. In some embodiments, combining the biological sample with the diluent or matrix resolving agent results in at least a 1, 2, 3, 4, 5, 6, 7, 8, 9, 10, 20, 30, 40, 50, 60, 70, 80, 90, 95, 99, or 100% decrease in viscoelasticity of the mucous matrix. In some embodiments, combining the biological sample with the diluent or matrix resolving agent results in at least a 1, 2, 3, 4, 5, 6, 7, 8, 9, 10, 20, 30, 40, 50, 60, 70, 80, 90, 95, 99, or 100% decrease in rigidity in the mucous matrix. In some embodiments, combining the biological sample with the diluent or matrix resolving agent results in at least a 1, 2, 3, 4, 5, 6, 7, 8, 9, 10, 20, 30, 40, 50, 60, 70, 80, 90, 95, 99, 100, 200, 300, 400, or 500% increase in porosity of the mucous matrix. In some embodiments, combining the biological sample with the diluent or matrix resolving agent results in at least a 1, 2, 3, 4, 5, 6, 7, 8, 9, 10, 20, 30, 40, 50, 60, 70, 80, 90, 95, 99, or 100% decrease in insolubility of the mucous matrix.

In some embodiments, a method or system of the disclosure has a higher detection rate for the target nucleic acid than a method or system that does not combine the biological sample with the diluent or matrix resolving agent. In some embodiments, a method or system of the disclosure has a detection rate at least 1, 2, 3, 4, 5, 6, 7, 8, 9, 10, 12, 15, 17, 20, 22, 25, 27, 30, 32, 35, 37, 40, 45, 50, 60, 70, 80, 90, 95, 99, or 100% higher for the target nucleic acid than a method or system that does not combine the biological sample with the diluent or matrix resolving agent. In some embodiments, a method or system of the disclosure has a detection rate at least 1-100, 1-80, 1-60, 1-50, 1-45, 1-40, 1-35, 1-30, 1-25, 1-20, 1-15, 1-10, 1-5, 5-100, 5-80, 5-60, 5-50, 5-45, 5-40, 5-35, 5-30, 5-25, 5-20, 5-15, 5-10, 10-100, 10-80, 10-60, 10-50, 10-45, 10-40, 10-35, 10-30, 10-25, 10-20, 10-15, 15-100, 15-80, 15-60, 15-50, 15-45, 15-40, 15-35, 15-30, 15-25, 15-20, 20-100, 20-80, 20-60, 20-50, 20-45, 20-40, 20-35, 20-30, 20-25, 25-100, 25-80, 25-60, 25-50, 25-45, 25-40, 25-35, 25-30, 30-100, 30-80, 30-60, 30-50, 30-45, 30-40, 30-35, 35-100, 35-80, 35-60, 35-50, 35-45, 35-40, 40-100, 40-80, 40-60, 40-50, 40-45, 45-100, 45-80, 45-60, 45-50, 50-100, 50-80, 50-60, 60-100, 60-80, or 80-100% higher for the target nucleic acid than a method or system that does not combine the biological sample with the diluent or matrix resolving agent.

In some embodiments, the diluent comprises liquid water. In some embodiments, the diluent comprises an organic solvent. In some embodiments, the diluent comprises a buffer, e.g., phosphate buffered saline or Tris. In some embodiments, a diluent for use herein has a pre-selected pH. In some embodiments, a diluent for use herein has a pH of about 8 or 8.1. In some embodiments, the diluent comprises amplification buffer solution. In some embodiments, the diluent does not comprise amplification buffer solution. In some embodiments, the diluent does not comprise an amplification reagent.

In some embodiments, combining a biological sample with a transfer fluid and a matrix resolving agent does not comprise combining the biological sample with an amplification reagent (e.g., the transfer fluid does not comprise an amplification reagent). In some embodiments, combining a biological sample with a diluent does not comprise combining the biological sample with an amplification reagent (e.g., the diluent does not comprise an amplification reagent). Without wishing to be bound by theory, the disclosure is directed in part to the discovery that, prior to amplifying a target nucleic acid that may be present in a biological sample (e.g., comprising a mucous matrix), it may be advantageous to combine a biological sample with a diluent (e.g., comprising a matrix resolving agent) or a matrix resolving agent. Combining a biological sample with a diluent (e.g., comprising a matrix resolving agent) or a matrix resolving agent may improve detection of a target nucleic acid, e.g., by altering a property of a mucous matrix that could interfere with amplification of a target nucleic acid.

In some embodiments, combining the biological sample with the diluent dilutes the biological sample at least 1.5×, 1.7×, 2×, 2.5×, 3×, 3.5×, 4×, 4.5×, 5×, 5.5×, 6×, 6.5×, 7×, 7.5×, 8×, 8.5×, 9×, 9.5×, 10×, 20×, 30×, 40×, 50×, 60×, 70×, 80×, 90×, 100×, 200×, 300×, 500×, or 1000× (and optionally no more than 2000×, 1000×, 100×, 50×, 10×, or 5×). In some embodiments, combining the biological sample with the diluent dilutes the biological sample 1.5×-2×, 1.5×-3×, 1.5×-4×, 1.5×-5×, 1.5×-10×, 1.5×-30×, 1.5×-50×, 1.5×-70×, 1.5×-100×, 1.5×-500×, 2×-3×, 2×-4×, 2×-5×, 2×-10×, 2×-30×, 2×-50×, 2×-70×, 2×-100×, 2×-500×, 3×-4×, 3×-5×, 3×-10×, 3×-30×, 3×- 50×, 3×-70×, 3×-100×, 4×-500×, 4×-5×, 4×-10×, 4×-30×, 4×-50×, 4×-70×, 4×-100×, 4×-500×, 5×-10×, 5×-30×, 5×-50×, 5×-70×, 5×-100×, 5×-500×, 10×-30×, 10×-50×, 10×-70×, 10×-100×, 10×-500×, 30×-50×, 30×-70×, 30×-100×, 30×-500×, 50×-70×, 50×-100×, 50×-500×, 70×-100×, 70×-500×, or 100×-500×.

In some embodiments, combining a biological sample with a diluent or transfer fluid produces a sample-containing fluid with a volume of 100 μl to 2000 μl, e.g., 100 μl to 1000 μl.

In some embodiments, a method of the disclosure further comprises a user obtaining a biological sample from a subject to be tested. In some embodiments, the user is the subject to be tested. In some embodiments, obtaining the biological sample comprises contacting a surface of a nasal cavity or an oral cavity of the subject with a sample collecting component. In some embodiments, the sample collecting component comprises a swab. In some embodiments, combining the biological sample with the diluent comprises depositing the biological sample in a first reaction tube comprising the diluent. In some embodiments, depositing the biological sample in the first reaction tube comprises agitating the swab in the reaction tube. In some embodiments, combining the biological sample with the diluent comprises adding diluent into a first reaction tube comprising the biological sample. In some embodiments, adding comprises pipetting and/or decanting. In some embodiments, combining the biological sample with the diluent comprises mixing the biological sample and the diluent. In some embodiments, mixing the biological sample and the diluent comprises inverting the first reaction tube and/or pipetting. In some embodiments, a method of the disclosure further comprises transferring the sample-containing fluid to a second reaction tube.

In some embodiments, a method of the disclosure further comprises applying heat to the sample-containing fluid. In some embodiments, a system of the disclosure further comprises a heater. In some embodiments, a system of the disclosure comprises electronic circuitry configured to control the heater to perform at least one heating protocol. In some embodiments, a heating protocol is comprised of maintaining at predetermined temperature for a predetermined amount of time. In some embodiments, a heating protocol is comprised of establishing and maintaining a plurality of predetermined temperatures, e.g., for predetermined amounts of time, e.g., sequentially. In some embodiments, the electronic circuitry is comprised of a processor programmed to control the heater to perform the at least one heating protocol. In some embodiments, a method of the disclosure comprises applying heat to the sample-containing fluid prior to amplifying the sample, prior to lysing a cell of the sample, and/or prior to adding a reagent to the sample (e.g., a matrix resolving agent, a lysis reagent, and/or an amplification reagent).

In some embodiments, the biological sample is from a subject. In some embodiments, a method of the disclosure further comprises identifying the subject as being infected with a pathogen based upon the presence of the target nucleic acid.

In some embodiments, a method of the disclosure further comprises amplifying the sample by permitting the sample-containing fluid to interact with at least one amplification reagent. In some embodiments, amplifying the sample occurs prior to contacting the lateral flow assay strip with the sample-containing fluid.

In some embodiments, the disclosure is directed to a method comprising combining a biological sample with a diluent to produce a sample-containing fluid; combining the sample-containing fluid with one or more lysis reagents; amplifying the sample by permitting the sample-containing fluid to interact with at least one amplification reagent; contacting a lateral flow assay strip (e.g., a lateral flow assay strip described herein) with the sample-containing fluid, and allowing the sample-containing fluid to move from a first end of the lateral flow assay strip to an indicator region. In some embodiments, the disclosure is directed to a method comprising combining a biological sample with a transfer fluid and a matrix resolving agent to produce a sample-containing fluid; combining the sample-containing fluid with one or more lysis reagents; amplifying the sample by permitting the sample-containing fluid to interact with at least one amplification reagent; contacting a lateral flow assay strip (e.g., a lateral flow assay strip described herein) with the sample-containing fluid; and allowing the sample-containing fluid to move from a first end of the lateral flow assay strip to an indicator region. In some embodiments, the disclosure is directed to a method comprising combining a biological sample with a diluent to produce a sample-containing fluid; combining the sample-containing fluid with one or more lysis reagents; amplifying the sample by permitting the sample-containing fluid to interact with at least one amplification reagent; and detecting amplification using a nucleic acid detection device (e.g., that measures amplification, e.g., by monitoring fluorescence, in real time). In some embodiments, the disclosure is directed to a method comprising combining a biological sample with a transfer fluid and a matrix resolving agent to produce a sample-containing fluid; combining the sample-containing fluid with one or more lysis reagents; amplifying the sample by permitting the sample-containing fluid to interact with at least one amplification reagent; and detecting amplification using a nucleic acid detection device (e.g., that measures amplification, e.g., by monitoring fluorescence, in real time).

### BRIEF DESCRIPTION OF THE DRAWINGS

FIG. 1 shows test results obtained using an exemplary device of the disclosure comprising a lateral flow assay strip, wherein the sample tested was treated using an exemplary diluent comprising an exemplary matrix resolving agent.

FIG. 2 shows a schematic of an exemplary method of processing a biological sample to reduce interference of a mucous matrix with target analyte detection.

FIG. 3 shows a graph of quantitative Loop-mediated Isothermal Amplification (qLAMP) amplification (mean cycle threshold (Ct) value) for samples containing nasal matrix and varying concentrations of Tween or EGTA.

FIG. 4 shows a graph of time to result for qLAMP reactions containing nasal matrix or buffer and a range of EDTA concentrations.

FIG. 5 shows a graph of time to result for qLAMP reactions containing nasal matrix or buffer and a range of EGTA concentrations.

FIG. 6 shows a table summarizing LAMP Lateral Flow Assay (LFA) detection data of SARS-CoV-2 target nucleic acid in samples containing heat-inactivated SARS-CoV-2 virus input mixed with nasal matrix and containing either EGTA at 200 μM or 1000 μM or EDTA at 1000 μM.

FIG. 7 shows a table of the rate at which endogenous RNase P failed to be amplified from samples (“invalid”) using either buffer containing EGTA at 200 μM (right) or 1000 μM EGTA (left)

FIG. 8 shows a table of the time to determination for qLAMP detecting SARS-CoV-2 target nucleic acid in nasal matrix-containing samples heated to a variety of temperatures (given in ° C.). Buffer or nasal material eluted in buffer was either (1) mixed with heat inactivated SARS-CoV-2 virus and then heated at the designated temperature for 5 minutes (“pre”) OR (2) heated at the designated temperature for 5 minutes, followed by the addition of heat inactivated SARS-CoV-2 virus (“post”). Sample preparation was followed in all cases by the subsequent addition of RT-LAMP reagents and measurement of SARs-CoV-2 amplification time (in minutes) by qLAMP. Nasal matrix samples were sourced from two separate donors, “p1” and “p2.”

FIG. 9 shows a table of the time to determination for qLAMP detecting SARS-CoV-2 target nucleic acid in nasal matrix-containing samples heated to a variety of temperatures (given in ° C.). Table labels are as described in FIG. 8, except nasal matrix samples were sourced from two additional donors, “p3” and “p4.”

FIG. 10 shows a table of the time to determination for detecting SARS-CoV-2 target nucleic acid by qLAMP in samples heated to a variety of temperatures (given in ° C.). Samples were either a nasal swab from a human (“Nasal”) or a dry swab (“Clean”). Lysis/amplification buffer was either buffer (“Detect buffer”) or nuclease free water (“NF water”). Nucleic acid templates were either encapsulated SARS-CoV-2 virus (“SeraCare”) or no template (“NTC”).

FIG. 11 shows a table of the time to determination in minutes for detecting SARS-CoV-2 target nucleic acid by qLAMP in samples heated to a variety of temperatures (given in ° C.) or room temperature (RT) after addition of lysis/amplification buffer lacking the indicated component (“Drop out”).

FIG. 12 shows a table of the time to determination for qLAMP detecting SARS-CoV-2 target nucleic acid (SeraCare synthetic encapsulated SARS-CoV-2 virus) in samples (either containing nasal matrix (Nasal) or not (Clean)) heated to a variety of temperatures (given in ° C.) or room temperature (RT) after addition of lysis/amplification buffer containing Tween (left table) or lacking Tween (right table).

FIG. 13 shows a table of the time to determination for qLAMP detecting SARS-CoV-2 target nucleic acid in samples containing various numbers of pooled swabs, with samples pre-heated to either 75° C. or room temperature (RT).

FIG. 14 shows a table of the time to determination for qLAMP detecting SARS-CoV-2 target nucleic acid in samples containing vaginal matrix diluted in various volumes of lysis/amplification buffer, with samples pre-heated to either 85° C. or room temperature (RT).

FIGS. 15A-15B show graphs of qLAMP reactions (FIG. 15A) and of time to result for qLAMP reactions (FIG. 15B) detecting target nucleic acid at varying sample-containing fluid pH for nasal matrix samples.

FIGS. 16A-16C show graphs of fluorescence over time in qLAMP reactions (FIGS. 16A and 16C) detecting SARS-CoV-2 target nucleic acid in 6 different nasal matrix samples, divided into aliquots where pH was measured and adjusted to buffer pH (FIG. 16A), with pH measurements for FIG. 16A shown in FIG. 16B, or where pH was adjusted to approximately 8 (FIG. 16C). Positive control detected amplification of RNase P (RP) endogenous gene target and no template control (NTC) contained no target nucleic acid.

FIG. 17 shows a graph of time to result for LAMP LFA detecting SARS-CoV-2 target nucleic acid in nasal matrix or buffer samples with varying concentrations of murine RNase inhibitor.

FIG. 18 shows tables summarizing LAMP LFA detection data of SARS-CoV-2 target nucleic acid in samples containing heat-inactivated SARS-CoV-2 virus (BEI) input, Seracare synthetic encapsulated SARS-CoV-2 virus, or both mixed with nasal matrix and containing no RNase, 0.1 U/μL RNase inhibitor, or 0.5 U/μL RNase inhibitor.

FIG. 19 shows a table summarizing LAMP LFA detection data of SARS-CoV-2 target nucleic acid in samples with (positive) or without (negative) heat-inactivated SARS-CoV-2 virus (BEI) input. RNase inhibitor is at a concentration of 0.1 U/uL in these samples, which were allowed to rest after mixing and prior to amplification for varying times.

FIG. 20 shows a table summarizing LAMP LFA detection data of RNase P (RP) gene nucleic acid in blood matrix samples diluted to varying degrees.

FIGS. 21A-21E show graphs of qLAMP fluorescence data detecting heat-inactivated SARS-CoV-2 virus spiked into blood matrix samples diluted to varying degrees. FIG. 21A uses EvaGreen fluorescent dye to track amplification, FIG. 21B uses FAM (6-carboxyfluorescein conjugated probe) to track amplification, FIG. 21C uses HEX™ conjugated probe to track amplification, FIG. 21D uses Texas Red conjugated probe to track amplification, and FIG. 21E uses Cy5 conjugated probe to track amplification.

FIG. 22 shows a graph of time to determination for qLAMP reactions detecting endogenous RP target nucleic acid in urine matrix samples diluted to varying degrees.

### DETAILED DESCRIPTION

The disclosure relates, in some aspects, to devices and methods for amplifying and/or detecting target analytes (e.g., nucleic acids) in a sample. The disclosure is based, in part, on methods that alter (e.g., decrease the level of or degrade) the mucous matrix present in a biological sample by combining the sample with a matrix resolving agent or a diluent (e.g., comprising a matrix resolving agent). In some embodiments, the diluent comprises a matrix resolving agent that alters the mucous matrix or complements the diluent's effect on the mucous matrix. The disclosure is also based, in part, on devices that combine a biological sample with a matrix resolving agent or diluent (e.g., comprising a matrix resolving agent). In some embodiments, the devices are used in a diagnostic test (e.g., a rapid, accurate, home diagnostic test). Without wishing to be bound by theory, biological samples obtained from subjects can comprise mucous matrices which can interfere with lysis of cells, dispersion of target analytes, amplification of nucleic acids, and/or flow of amplified nucleic acid along a lateral flow assay strip in an immunoassay device. Contacting a mucous matrix or sample comprising the same with a matrix resolving agent or a diluent (e.g., comprising a matrix resolving agent) can alter one or more properties of the mucous matrix and/or make one or more biological components (e.g., target analytes) accessible to downstream processing. In some embodiments, devices and methods described herein represent an improvement over currently available amplification and detection methods because they increase the detection rate of target analytes (e.g., nucleic acids) from samples comprising a mucous matrix (e.g., a sample from a nasal cavity).

As used herein, a mucous matrix refers to a collection of sample components comprising a plurality of glycoproteins which, when hydrated, have a viscosity greater than water. In some embodiments, a mucous matrix impedes (e.g., slows or prevents) movement of biological components (e.g., cells, nucleic acids, or proteins) into and out of the mucous matrix. In some embodiments, a mucous matrix comprises one or more mucin proteins, e.g., a secreted or gel forming mucin, polymeric mucin, or non-secreted surface-bound mucin. In some embodiments, a mucous matrix comprises one or more of MUC7, MUC8, MUC2, MUC5AC, MUC5B, MUC19, MUC1, MUC4, MUC13, MUC16, MUC20, MUC21, or MUC22. In some embodiments, a mucous matrix comprises a proteoglycan. In some embodiments, a mucous matrix comprises one or more additional proteins, e.g., fibrillar collagens, elastin, fibronectin, laminin, nidogen. In some embodiments, a mucous matrix comprises an enzyme, e.g., a nuclease, e.g., an RNase. In some embodiments, a mucous matrix comprises one or more cells, e.g., cells comprising a target analyte which must be lysed to access the target analyte and/or cells not comprising a target analyte. In some embodiments, a mucous matrix comprises one or more ions (e.g., a dicationic metal). Without wishing to be bound by theory, a mucous matrix present in a biological sample may inhibit detection of a target analyte by one or more mechanisms. For example, a mucous matrix may comprise a sample component that promotes digestion of a target nucleic acid (e.g., an RNase) thereby inhibiting detection of the target nucleic acid. As a further example, the same or a different mucous matrix may comprise a sample component that increases viscosity of the sample and impedes movement of a target analyte.

Many tissues in a subject, e.g., a human subject, produce mucous matrices and may be accessed to obtain a biological sample for use in a method or device of the disclosure. The mucous matrix of a sample may be referred to herein by the orifice or tissue from which the sample was taken. For example, a mucous matrix in a biological sample from the nasal cavity (e.g., an anterior nares sample) may be referred to as a nasal matrix. In some embodiments, a mucous matrix in a biological sample is a nasal matrix. As a further example, a mucous matrix in a biological sample from the oral cavity (e.g., a cheek swab sample) may be referred to an oral matrix. In some embodiments, a mucous matrix in a biological sample is an oral matrix. As a further example, a mucous matrix in a biological sample from the throat or pharynx may be referred to as a pharyngeal matrix. In some embodiments, a mucous matrix in a biological sample is a pharyngeal matrix. As a further example, a mucous matrix in a biological sample from the esophagus may be referred to as an esophageal matrix. In some embodiments, a mucous matrix in a biological sample is an esophageal matrix. As a further example, a mucous matrix in a biological sample from the ear may be referred to as an aural matrix. In some embodiments, a mucous matrix in a biological sample is an aural matrix. As a further example, a mucous matrix in a biological sample from the vagina may be referred to as a vaginal matrix. In some embodiments, a mucous matrix in a biological sample is a vaginal matrix. As a further example, a mucous matrix in a biological sample from the blood may be referred to as a blood matrix. In some embodiments, a mucous matrix in a biological sample is a blood matrix. As a further example, a mucous matrix in a biological sample from the urine may be referred to as a urine matrix. In some embodiments, a mucous matrix in a biological sample is a urine matrix.

### Diluents and Matrix Resolving Agents

Aspects of the disclosure relate to methods comprising combining a biological sample with a matrix resolving agent or a diluent (e.g., comprising a matrix resolving agent). In some embodiments, a biological sample applicable to a method or device described herein comprises a mucous matrix. In some embodiments, a biological sample combined with a matrix resolving agent or a diluent (e.g., comprising a matrix resolving agent), e.g., to form a sample-containing fluid, is applied to a rapid testing device or a method for detecting a nucleic acid (e.g., a rapid test for a nucleic acid). In some embodiments, a method of the disclosure comprises applying heat to the sample-containing fluid prior to amplifying the sample, prior to lysing a cell of the sample, prior to applying the sample-containing fluid to a rapid testing device, and/or prior to adding a reagent to the sample (e.g., a matrix resolving agent, a lysis reagent, and/or an amplification reagent). In some embodiments, the rapid testing device or method for detecting a nucleic acid (e.g., a rapid test for a nucleic acid) is a rapid testing device or method described herein. In other embodiments, the rapid testing device or method for detecting a nucleic acid (e.g., a rapid test for a nucleic acid) is a rapid testing device or method known in the art. Rapid testing devices and methods for detecting a target nucleic acid described herein for use with the matrix resolving agents, diluents, and steps combining a biological sample with a matrix resolving agent or a diluent (e.g., comprising a matrix resolving agent) of the disclosure are exemplary only and not intended to limit the disclosure in any way.

### Matrix Resolving Agents

As used herein, a matrix resolving agent refers to an agent that performs one or more of the following functions when contacted with a sample comprising a mucous matrix (e.g., a nasal matrix): decreases viscosity, decreases viscoelasticity, decreases rigidity; increases porosity; decreases insolubility; alters topography; or cleaves, digests, binds (e.g., chelates), immobilizes, inactivates, or otherwise decreases the concentration of a protein, glycan, or proteoglycan or a cofactor (e.g., an ion) bound by any thereof. A matrix resolving agent may promote release of cells and/or cellular components (e.g., a target nucleic acid) from a mucous matrix, e.g., as measured by increased detection rates of an assay for said cells or cellular components, e.g., as described in Example 1. In some embodiments, a matrix resolving agent comprises a reducing agent. In some embodiments the reducing agent comprises DTT (dithiothreitol), glutathione, DTE (dithioerythritol), TCEP, 2-mercaptoethanol, or a combination thereof. In some embodiments, a matrix resolving agent comprises a chelating agent, e.g., EGTA or EDTA. In some embodiments, the chelating agent binds to a cation in a biological sample or sample-containing fluid and/or inactivates an interfering enzyme. For example, in some embodiments an interfering enzyme utilizes a cation cofactor and binding of cations by chelating agents inactivates the interfering enzyme. In some embodiments, the chelating agent is EGTA. In some embodiments, a matrix resolving agent comprises a protein, e.g., an enzyme. In some embodiments, the protein, e.g., enzyme, is a recombinant protein. In some embodiments, the enzyme is a metalloproteinase, e.g., a matrix metalloproteinase (MMP) or a disintegrin and metalloproteinase with thromospondin motifs (ADAMTS) family protein. In some embodiments, the MMP or ADAMTS is an enzyme from Table 1 of Lu et al. Cold Spring Harb Perspect Biol 2011; 3:a005058, e.g., MMP-1, MMP-2, MMP-3, MMP-7, MMP-8, MMP-14, ADAMTS-1, ADAMTS-4, ADAMTS-2, ADAMTS-13, ADAM-TS 7, or ADAMTS-16. In some embodiments, the enzyme is capable of degrading or digesting glycoproteins. In some embodiments, the enzyme is a nuclease, e.g., capable of degrading or digesting DNA. In some embodiments, the protein is an inhibitor of an enzyme, e.g., an RNase inhibitor. In some embodiments, the Rnase inhibitor is a murine Rnase inhibitor. In some embodiments, the protein is a protease, e.g., proteinase K. In some embodiments, a matrix resolving agent comprises a mucolytic agent, e.g., N-acetylcysteine (NAC), dornase alfa, or thymosin β4. Mucolytic agents are agents, e.g., drugs, that resolve mucous matrices. Resolving a mucous matrix (or sample comprising a mucous matrix) or resolution of a mucous matrix (or sample comprising a mucous matrix) refers to a process or step that alters a property of a mucous matrix. In some embodiments, resolving or resolution alters a property of a mucous matrix such that one or more biological components (e.g., cells, nucleic acids, or proteins) associated with the mucous matrix (or the sample comprising the mucous matrix) can freely dissociate from the mucous matrix. In some embodiments, resolving or resolution alters a property of a mucous matrix such that one or more biological components (e.g., cells, nucleic acids, or proteins) associated with the mucous matrix can participate in a downstream process (e.g., a lysis step, amplification step, or a step contacting the component to a lateral flow assay strip). In some embodiments, resolving or resolution thins or disperses a mucous matrix, e.g., by decreasing the viscosity or viscoelasticity of the matrix. In some embodiments, resolving or resolution depolymerizes a polymer comprised in the mucous matrix, e.g., actin or DNA. In some embodiments, resolving or resolution cleaves, digests, or inactivates a protein, glycan, or proteoglycan (e.g., an interfering enzyme) present in a mucous matrix (e.g., in a biological sample or sample-containing fluid (e.g., in a cell comprised therein)).

In some embodiments, a matrix resolving agent is provided (e.g., in a method or device described herein) in solid form (e.g., lyophilized, dried, crystallized, air jetted). In some embodiments, a matrix resolving agent is provided (e.g., in a method or device described herein) in aqueous form.

The disclosure is directed, in part, to methods that utilize a plurality of matrix resolving agents. In some embodiments, a method comprises combining a biological sample or sample-containing fluid with at least one matrix resolving agent, e.g., 1, 2, 3, 4, 5, 6, 7, 8, 9, or 10 matrix resolving agents. In some embodiments, a method comprises combining a biological sample or sample-containing fluid with at least two matrix resolving agent, e.g., 2, 3, 4, 5, 6, 7, 8, 9, or 10 matrix resolving agents. In some embodiments, a method comprises combining a biological sample or sample-containing fluid with at least three matrix resolving agent, e.g., 3, 4, 5, 6, 7, 8, 9, or 10 matrix resolving agents. In some embodiments, a method comprises combining a biological sample or sample-containing fluid with a reducing agent (e.g., DTT) and a chelator (e.g., EGTA). In some embodiments, a method comprises combining a biological sample or sample-containing fluid with a reducing agent (e.g., DTT) and an inhibitor of an enzyme (e.g., an RNase inhibitor). In some embodiments, a method comprises combining a biological sample or sample-containing fluid with a chelator (e.g., EGTA) and an inhibitor of an enzyme (e.g., an RNase inhibitor). In some embodiments, a method comprises combining a biological sample or sample-containing fluid with a reducing agent (e.g., DTT), an inhibitor of an enzyme (e.g., an RNase inhibitor), and a chelator (e.g., EGTA). In some embodiments, one, a plurality, or all of the matrix resolving agents are comprised within a diluent which is also combined with the biological sample or sample-containing fluid.

The disclosure is directed, in part, to compositions (e.g., rapid test systems and sample preparation mixtures) comprising a plurality of matrix resolving agents. In some embodiments, a composition (e.g., a rapid test system or sample preparation mixture) comprises at least one matrix resolving agent, e.g., 1, 2, 3, 4, 5, 6, 7, 8, 9, or 10 matrix resolving agents. In some embodiments, a composition (e.g., a rapid test system or sample preparation mixture) comprises at least two matrix resolving agents, e.g., 2, 3, 4, 5, 6, 7, 8, 9, or 10 matrix resolving agents. In some embodiments, a composition (e.g., a rapid test system or sample preparation mixture) comprises at least three matrix resolving agents, e.g., 3, 4, 5, 6, 7, 8, 9, or 10 matrix resolving agents. In some embodiments, a composition (e.g., a rapid test system or sample preparation mixture) comprises a reducing agent (e.g., DTT) and a chelator (e.g., EGTA). In some embodiments, a composition (e.g., a rapid test system or sample preparation mixture) comprises a reducing agent (e.g., DTT) and an inhibitor of an enzyme (e.g., an RNase inhibitor). In some embodiments, a composition (e.g., a rapid test system or sample preparation mixture) comprises a chelator (e.g., EGTA) and an inhibitor of an enzyme (e.g., an RNase inhibitor). In some embodiments, a composition (e.g., a rapid test system or sample preparation mixture) comprises a reducing agent (e.g., DTT), an inhibitor of an enzyme (e.g., an RNase inhibitor), and a chelator (e.g., EGTA). In some embodiments, one, a plurality, or all of the matrix resolving agents are comprised within a diluent. In some embodiments, a composition (e.g., a rapid test system or sample preparation mixture) comprises a diluent which comprises one, a plurality, or all of the matrix resolving agents.

### Diluents

In some embodiments, a biological sample, e.g., comprising a mucous matrix, is combined with a diluent to produce a sample-containing fluid, e.g., prior to a lysis step, an amplification step, or both. In some embodiments, combining a biological sample comprising a mucous matrix with a diluent to produce a sample-containing fluid dilutes the mucous matrix or at least one component thereof. Without wishing to be bound by theory, combining a biological sample comprising a mucous matrix with a diluent is thought to improve one or more physico-chemical properties of the mucous matrix and/or sample-containing fluid. In some embodiments, improvement of one or more physico-chemical properties improves processing of a biological sample in downstream steps, e.g., in one or more (e.g., all) of a lysis, amplification, or detection step. Physico-chemical properties improved by dilution of the mucous matrix include, but are not limited to: decreased viscosity, decreased viscoelasticity, decreased rigidity; increased porosity; decreased insolubility; or altered topography of the mucous matrix. Such effects are thought to improve one or more (e.g., all) of lysis of cells that may be contained in the biological sample, amplification of nucleic acids (e.g., a target nucleic acid) that may be contained in the biological sample, or detection of a target nucleic acid that may be contained in the biological sample. In some embodiments, the diluent comprises liquid water. In some embodiments, the diluent comprises an organic solvent. In some embodiments, the diluent comprises a buffer, e.g., phosphate buffered saline or Tris. In some embodiments, the diluent comprises one or more components of a downstream process, e.g., a lysis step or an amplification step. In some embodiments, the diluent comprises amplification buffer solution. In some embodiments, the diluent comprises one or more lysis reagents.

In some embodiments, combining a biological sample, e.g., comprising a mucous matrix, with a diluent dilutes the biological sample, the mucous matrix, or at least one component of either thereof by at least 1.5×, 1.7×, 2×, 2.5×, 3×, 3.5×, 4×, 4.5×, 5×, 5.5×, 6×, 6.5×, 7×, 7.5×, 8×, 8.5×, 9×, 9.5×, 10×, 20×, 30×, 40×, 50×, 60×, 70×, 80×, 90×, 100×, 200×, 300×, 500×, or 1000× (and optionally no more than 2000×, 1000×, 100×, 50×, 10×, or 5×). In some embodiments, combining a biological sample, e.g., comprising a mucous matrix, with a diluent dilutes the biological sample, the mucous matrix, or at least one component of either thereof by 1.5×-2×, 1.5×-3×, 1.5×-4×, 1.5×-5×, 1.5×-10×, 1.5×-30×, 1.5×-50×, 1.5×-70×, 1.5×-100×, 1.5×-500×, 2×-3×, 2×-4×, 2×-5×, 2×-10×, 2×-30×, 2×-50×, 2×-70×, 2×-100×, 2×-500×, 3×-4×, 3×-5×, 3×-10×, 3×-30×, 3×-50×, 3×-70×, 3×-100×, 4×-500×, 4×-5×, 4×-10×, 4×-30×, 4×-50×, 4×-70×, 4×-100×, 4×-500×, 5×-10×, 5×-30×, 5×-50×, 5×-70×, 5×-100×, 5×-500×, 10×-30×, 10×-50×, 10×-70×, 10×-100×, 10×-500×, 30×-50×, 30×-70×, 30×-100×, 30×-500×, 50×-70×, 50×-100×, 50×-500×, 70×-100×, 70×-500×, or 100×-500×. As used herein, ‘#x’ in the context of dilution refers to the fold change in concentration of a sample constituent. For example, diluting the mucous matrix or at least one component thereof by at least 2× refers to decreasing the concentration of the mucous matrix or at least one component thereof in the resulting sample-containing fluid by at least half.

In some embodiments, combining a biological sample, e.g., comprising a mucous matrix, with a diluent produces a sample-containing fluid with a volume of at least about 100 μl, at least about 200 μl, at least about 300 μl, at least about 400 μl, at least about 500 μl, at least about 600 μl, at least about 700 μl, at least about 800 μl, at least about 900 μl, or at least about 1000 μl. In some embodiments, combining a biological sample, e.g., comprising a mucous matrix, with a diluent produces a sample-containing fluid with a volume of 100-1000 μl, 200-1000 μl, 300-1000 μl, 400-1000 μl, 500-1000 μl, 600-1000 μl, 700-1000 μl, 800-1000 μl, 900-1000 μl, 100-900 μl, 200-900 μl, 300-900 μl, 400-900 μl, 500-900 μl, 600-900 μl, 700-900 μl, 800-900 μl, 100-800 μl, 200-800 μl, 300-800 μl, 400-800 μl, 500-800 μl, 600-800 μl, 700-800 μl, 100-700 μl, 200-700 μl, 300-700 μl, 400-700 μl, 500-700 μl, 600-700 μl, 100-600 μl, 200-600 μl, 300-600 μl, 400-600 μl, 500-600 μl, 100-500 μl, 200-500 μl, 300-500 μl, 400-500 μl, 100-400 μl, 200-400 μl, 300-400 μl, 100-300 μl, 200-300 μl, or 100-200 μl. Without wishing to be bound by theory, the disclosure is directed, in part, to the idea that methods forming a sample-containing fluid having a volume above a threshold value or within the described ranges may more effectively detect a target nucleic acid than a method forming a sample-containing fluid having a volume below the threshold value or outside the described ranges. For example, in some embodiments, target nucleic acids present at low, e.g., difficult to detect, concentrations in a biological sample or present with mucous matrices are more effectively detected (e.g., will be detected with fewer false positives and/or false negatives) by a method utilizes a large sample volume (above a threshold value or within the described ranges) than a small sample volume. In some embodiments, the sample volume is established by addition of a suitable volume of diluent. In some embodiments, the sample volume is maintained through one, two, or all of pre-heating, lysis, and amplification.

In some embodiments, the diluent has a relatively neutral pH. In some embodiments, the diluent establishes and/or maintains a relatively neutral pH in the sample-containing fluid. In some embodiments, the diluent has a basic pH. In some embodiments, the diluent establishes and/or maintains a basic pH in the sample-containing fluid and/or neutralizes an acidic pH in the biological sample. In some embodiments, the diluent comprises one or more buffers. Non-limiting examples of suitable buffers include phosphate-buffered saline (PBS) and Tris. In some embodiments, the diluent has a pH in a range from 5.0 to 6.0, 5.0 to 7.0, 5.0 to 8.0, 5.0 to 9.0, 6.0 to 7.0, 6.0 to 8.0, 6.0 to 9.0, 7.0 to 8.0, 7.0 to 9.0, or 8.0 to 9.0. In some embodiments, the diluent has a pH of about 7, about 7.5, about 7.75, about 8, about 8.1, about 8.2, about 8.3, about 8.4, about 8.5, about 8.6, about 8.7, about 8.8, about 8.9, about 9, about 9.1, about 9.2, or about 9.3. In some embodiments, the diluent has a pH selected to produce a sample-containing fluid (e.g., an amplification mixture) with a pH of about 8, about 8.1, about 8.2, or about 8.3. In some embodiments, the diluent has a pH selected to produce a sample-containing fluid (e.g., an amplification mixture) with a pH of from about 8 to about 8.1, e.g., 8-8.1 or 8.05-8.1. In some embodiments a method described herein comprises forming a sample-containing fluid having a pH described herein. Without wishing to be bound by theory, a slightly basic pH, e.g., a pH from 7-9.3 described herein, may improve target nucleic acid detection by a method described herein relative to a method employing a sample-containing fluid or diluent pH outside the recited ranges. As used herein, “about” refers to a range around a value of ±10%, ±9%, ±8%, ±7%, ±6%, ±5%, ±4%, ±3%, ±2%, or ±1% (e.g., in some embodiments, a pH of about 8 includes pH 7.2-8.8 (i.e., ±10%); in other embodiments, a pH of about 8 includes pH 7.92-8.08 (i.e., ±1%)).

In some embodiments, a diluent comprises a matrix resolving agent. Without wishing to be bound by theory, it is thought that the effects of combining a biological sample comprising a mucous matrix with a diluent and the effects of combining a biological sample comprising a mucous matrix with a matrix resolving agent are complementary. In some embodiments, the beneficial effects of combining a diluent with a matrix resolving agent are additive. In some embodiments, the improvement to a physico-chemical property or to a downstream process, e.g., a lysis step or an amplification step, obtained from using a diluent comprising a matrix resolving agent is greater than the corresponding improvement obtained from using a diluent alone or a matrix resolving agent alone. In some embodiments, a synergistic improvement to a physico-chemical property or to a downstream process, e.g., a lysis step or an amplification step, is obtained from using a diluent comprising a matrix resolving agent, e.g., an improvement greater than the expected additive improvement from use of a diluent or a matrix resolving agent alone.

In some embodiments, a matrix resolving agent is combined with a biological sample, e.g., comprising a mucous matrix, without combining the biological sample with a diluent. In some embodiments, the matrix resolving agent is added to the biological sample in solid form. In some embodiments, a biological sample is deposited into a transfer fluid, e.g., in a device described herein or a reaction tube. In some embodiments, the matrix resolving agent (e.g., in solid form) is combined with a biological sample in a transfer fluid. In some embodiments, the matrix resolving agent and transfer fluid are combined with the biological sample. In some embodiments, the matrix resolving agent and transfer fluid are combined prior to combining either with the biological sample. A transfer fluid, as used herein, may be any liquid (e.g., solution) suitable for a matrix resolving agent to act upon a biological sample (e.g., a mucous matrix comprised in a biological sample). In some embodiments, the transfer fluid comprises one or more components of a downstream process, e.g., a lysis step or an amplification step. In some embodiments, the transfer fluid comprises amplification buffer solution. In some embodiments, the transfer fluid comprises one or more lysis reagents.

In some embodiments, combining the biological sample with the diluent and/or matrix resolving agent results in at least a 1, 2, 3, 4, 5, 6, 7, 8, 9, 10, 20, 30, 40, 50, 60, 70, 80, 90, 95, 99, or 100% decrease in viscosity of the mucous matrix or sample-containing fluid. In some embodiments, combining the biological sample with the diluent or matrix resolving agent results in at least a 1, 2, 3, 4, 5, 6, 7, 8, 9, 10, 20, 30, 40, 50, 60, 70, 80, 90, 95, 99, or 100% decrease in viscoelasticity of the mucous matrix or sample-containing fluid. In some embodiments, combining the biological sample with the diluent or matrix resolving agent results in at least a 1, 2, 3, 4, 5, 6, 7, 8, 9, 10, 20, 30, 40, 50, 60, 70, 80, 90, 95, 99, or 100% decrease in rigidity in the mucous matrix or sample-containing fluid. In some embodiments, combining the biological sample with the diluent or matrix resolving agent results in at least a 1, 2, 3, 4, 5, 6, 7, 8, 9, 10, 20, 30, 40, 50, 60, 70, 80, 90, 95, 99, 100, 200, 300, 400, or 500% increase in porosity of the mucous matrix or sample-containing fluid. In some embodiments, combining the biological sample with the diluent or matrix resolving agent results in at least a 1, 2, 3, 4, 5, 6, 7, 8, 9, 10, 20, 30, 40, 50, 60, 70, 80, 90, 95, 99, or 100% decrease in insolubility in of the mucous matrix. In some embodiments, combining the biological sample with the diluent or matrix resolving agent results in at least a 1, 2, 3, 4, 5, 6, 7, 8, 9, 10, 20, 30, 40, 50, 60, 70, 80, 90, 95, 99, or 100% decrease in activity of an interfering enzyme in the biological sample or the sample-containing fluid. Physico-chemical properties of mucous matrices and changes thereof may be determined by methods known to those of skill in the art, e.g., by methods described by Atanosova and Reznikov. Respir Res. 2019 Nov. 21; 20(1):261; and Lu et al. Cold Spring Harb Perspect Biol. 2011 Dec. 1; 3(12):a005058.

In some embodiments, combining comprises depositing the biological sample in a first reaction tube comprising the diluent and/or matrix resolving agent or into a device (e.g., a chamber of a device described herein) or reaction tube comprising the diluent and/or matrix resolving agent. In some embodiments, combining comprises depositing the biological sample in a first reaction tube or into a device (e.g., a chamber of a device described herein) and then adding diluent to the device or reaction tube. In some embodiments, adding comprises mixing the biological sample and the diluent and/or matrix resolving agent. In some embodiments, adding comprises pipetting and/or decanting. In some embodiments, the biological sample is deposited into the lysis chamber of a device (e.g., a rapid test device) described herein. In some embodiments, the biological sample is deposited into the sample preparation chamber of a device (e.g., a rapid test device) described herein.

The disclosure is directed, in part, to a method for detecting a target nucleic acid comprising combining a biological sample with a matrix resolving agent or diluent (e.g., comprising a matrix resolving agent) to produce a sample-containing fluid. In some embodiments, the combining occurs prior to one or more downstream steps of the method. In some embodiments, the combining occurs prior to contacting a lateral flow assay strip having a first end and a second end with the sample-containing fluid. In some embodiments, the combining occurs prior to combining the biological sample or sample-containing fluid with one or more lysis reagents (e.g., in a lysis chamber of a device described herein). In some embodiments, the combining occurs prior to amplifying the sample, e.g., by permitting the sample-containing fluid to interact with at least one amplification reagent, e.g., in an amplification chamber of a test device. In some embodiments, a method for detecting a target nucleic acid comprising combining a biological sample with a matrix resolving agent or diluent (e.g., comprising a matrix resolving agent) to produce a sample-containing fluid has a higher detection rate for the target nucleic acid than an otherwise similar method that does not combine the biological sample with a matrix resolving agent or diluent. In some embodiments, a method for detecting a target nucleic acid comprises combining a biological sample with a matrix resolving agent or diluent (e.g., comprising a matrix resolving agent) to produce a sample-containing fluid has a lower false negative rate for the target nucleic acid than an otherwise similar method that does not combine the biological sample with a matrix resolving agent or diluent. In some embodiments, the method has a detection rate at least 1, 2, 3, 4, 5, 6, 7, 8, 9, 10, 12, 15, 17, 20, 22, 25, 27, 30, 32, 35, 37, 40, 45, 50, 60, 70, 80, 90, 95, 99, or 100% higher for the target nucleic acid than an otherwise similar method that does not combine the biological sample with a matrix resolving agent or diluent. In some embodiments, the method has a detection rate at least 1-100, 1-80, 1-60, 1-50, 1-45, 1-40, 1-35, 1-30, 1-25, 1-20, 1-15, 1-10, 1-5, 5-100, 5-80, 5-60, 5-50, 5-45, 5-40, 5-35, 5-30, 5-25, 5-20, 5-15, 5-10, 10-100, 10-80, 10-60, 10-50, 10-45, 10-40, 10-35, 10-30, 10-25, 10-20, 10-15, 15-100, 15-80, 15-60, 15-50, 15-45, 15-40, 15-35, 15-30, 15-25, 15-20, 20-100, 20-80, 20-60, 20-50, 20-45, 20-40, 20-35, 20-30, 20-25, 25-100, 25-80, 25-60, 25-50, 25-45, 25-40, 25-35, 25-30, 30-100, 30-80, 30-60, 30-50, 30-45, 30-40, 30-35, 35-100, 35-80, 35-60, 35-50, 35-45, 35-40, 40-100, 40-80, 40-60, 40-50, 40-45, 45-100, 45-80, 45-60, 45-50, 50-100, 50-80, 50-60, 60-100, 60-80, or 80-100% higher for the target nucleic acid than a method that does not combine the biological sample with a diluent or a matrix resolving agent. In some embodiments, the method has a false negative rate at least 1, 2, 3, 4, 5, 6, 7, 8, 9, 10, 12, 15, 17, 20, 22, 25, 27, 30, 32, 35, 37, 40, 45, 50, 60, 70, 80, 90, 95, 99, or 100% lower for the target nucleic acid than an otherwise similar method that does not combine the biological sample with a matrix resolving agent or diluent. In some embodiments, the method has a false negative at least 1-100, 1-80, 1-60, 1-50, 1-45, 1-40, 1-35, 1-30, 1-25, 1-20, 1-15, 1-10, 1-5, 5-100, 5-80, 5-60, 5-50, 5-45, 5-40, 5-35, 5-30, 5-25, 5-20, 5-15, 5-10, 10-100, 10-80, 10-60, 10-50, 10-45, 10-40, 10-35, 10-30, 10-25, 10-20, 10-15, 15-100, 15-80, 15-60, 15-50, 15-45, 15-40, 15-35, 15-30, 15-25, 15-20, 20-100, 20-80, 20-60, 20-50, 20-45, 20-40, 20-35, 20-30, 20-25, 25-100, 25-80, 25-60, 25-50, 25-45, 25-40, 25-35, 25-30, 30-100, 30-80, 30-60, 30-50, 30-45, 30-40, 30-35, 35-100, 35-80, 35-60, 35-50, 35-45, 35-40, 40-100, 40-80, 40-60, 40-50, 40-45, 45-100, 45-80, 45-60, 45-50, 50-100, 50-80, 50-60, 60-100, 60-80, or 80-100% lower for the target nucleic acid than a method that does not combine the biological sample with a diluent or a matrix resolving agent.

Pre-Heating of Biological Samples and/or Sample-Containing Fluids

The disclosure is directed, in part, to a method for detecting a target nucleic acid comprising heating a biological sample or sample-containing fluid. Without wishing to be bound by theory, heating a biological sample or sample-containing fluid may resolve a mucous matrix in the biological sample or sample-containing fluid. In some embodiments, resolving or resolution alters a property of a mucous matrix such that one or more biological components (e.g., cells, nucleic acids, or proteins) associated with the mucous matrix (or the sample comprising the mucous matrix) can freely dissociate from the mucous matrix. In some embodiments, resolving or resolution alters a property of a mucous matrix such that one or more biological components (e.g., cells, nucleic acids, or proteins) associated with the mucous matrix can participate in a downstream process (e.g., a lysis step, amplification step, or a step contacting the component to a lateral flow assay strip). In some embodiments, resolving or resolution thins or disperses a mucous matrix, e.g., by decreasing the viscosity or viscoelasticity of the matrix. In some embodiments, resolving or resolution depolymerizes a polymer comprised in the mucous matrix, e.g., actin or DNA. In some embodiments, resolving or resolution cleaves, digests, or inactivates a protein, glycan, or proteoglycan (e.g., an interfering enzyme) present in a mucous matrix (e.g., in a biological sample or sample-containing fluid (e.g., in a cell comprised therein)). As used herein, heating encompasses both applying or removing heat from a sample; e.g., a sample may be heated to a series of temperatures (e.g., room temperature, 85° C., and 60° C.), wherein heat is removed (e.g., by cooling) to ‘heat’ the sample from 85° C. to 60° C. In some embodiments, heat is removed by cooling, e.g., passive cooling wherein a sample is incubated in a lower temperature (e.g., room temperature) environment.

In some embodiments, heating a biological sample or sample-containing fluid results in at least a 1, 2, 3, 4, 5, 6, 7, 8, 9, 10, 20, 30, 40, 50, 60, 70, 80, 90, 95, 99, or 100% decrease in viscosity of the mucous matrix or sample-containing fluid. In some embodiments, heating a biological sample or sample-containing fluid results in at least a 1, 2, 3, 4, 5, 6, 7, 8, 9, 10, 20, 30, 40, 50, 60, 70, 80, 90, 95, 99, or 100% decrease in viscoelasticity of the mucous matrix or sample-containing fluid. In some embodiments, heating a biological sample or sample-containing fluid results in at least a 1, 2, 3, 4, 5, 6, 7, 8, 9, 10, 20, 30, 40, 50, 60, 70, 80, 90, 95, 99, or 100% decrease in rigidity in the mucous matrix or sample-containing fluid. In some embodiments, heating a biological sample or sample-containing fluid results in at least a 1, 2, 3, 4, 5, 6, 7, 8, 9, 10, 20, 30, 40, 50, 60, 70, 80, 90, 95, 99, 100, 200, 300, 400, or 500% increase in porosity of the mucous matrix or sample-containing fluid. In some embodiments, heating a biological sample or sample-containing fluid results in at least a 1, 2, 3, 4, 5, 6, 7, 8, 9, 10, 20, 30, 40, 50, 60, 70, 80, 90, 95, 99, or 100% decrease in insolubility in of the mucous matrix. In some embodiments, heating a biological sample or sample-containing fluid results in at least a 1, 2, 3, 4, 5, 6, 7, 8, 9, 10, 20, 30, 40, 50, 60, 70, 80, 90, 95, 99, or 100% decrease in activity of an interfering enzyme in the biological sample or the sample-containing fluid. Physico-chemical properties of mucous matrices and changes thereof may be determined by methods known to those of skill in the art, e.g., by methods described by Atanosova and Reznikov. Respir Res. 2019 Nov. 21; 20(1):261; and Lu et al. Cold Spring Harb Perspect Biol. 2011 Dec. 1; 3(12):a005058.

In some embodiments, a method for detecting a target nucleic acid comprising applying heat to a biological sample or sample-containing fluid prior to one or more lysis or amplification reagents being present in the sample-containing fluid. In some embodiments, the heat is applied to a biological sample or sample-containing fluid prior to any amplification reagents being present in the sample-containing fluid. In some embodiments, the heat is applied to a biological sample or sample-containing fluid prior to any lysis reagents being present in the sample-containing fluid. In some embodiments, a method for detecting a target nucleic acid comprises applying heat to the biological sample prior to addition of diluent to the biological sample. In some embodiments, a method for detecting a target nucleic acid comprises applying heat to the biological sample prior to addition of a matrix resolving agent to the biological sample. In some embodiments, a method for detecting a target nucleic acid comprises applying heat to the sample-containing fluid prior to amplifying the sample. In some embodiments, a method for detecting a target nucleic acid comprises applying heat to the sample-containing fluid prior to lysing a cell of the sample. In some embodiments, a method for detecting a target nucleic acid comprises applying heat to the sample-containing fluid prior to adding a reagent to the sample (e.g., a matrix resolving agent, a lysis reagent, and/or an amplification reagent). For example, in some embodiments, a biological sample is mixed with a diluent and/or matrix resolving agent to form a sample-containing fluid, heat is applied (e.g., to resolve a mucous matrix), and then one or more reagents (e.g., lysis reagents and/or amplification reagents) are added. Without wishing to be bound by theory, heating a biological sample or sample-containing fluid prior to a downstream step (such as, e.g., addition of lysis reagents or amplification reagents) may improve a method of detecting a target nucleic acid (e.g., as measured by increased detection rates, e.g., as described in Example 1) by cleaving, digesting, or inactivating one or more interfering enzymes, e.g., prior to the interfering enzymes contacting an amplification reagent and/or prior to the interfering enzymes being released from a cell. Alternately or additionally, heating a biological sample or sample-containing fluid prior to a downstream step (such as, e.g., addition of lysis reagents or amplification reagents) may improve a method of detecting a target nucleic acid (e.g., as measured by increased detection rates, e.g., as described in Example 1) by not inactivating a lysis reagent or amplification reagent, e.g., that is sensitive to heat or the presence of another reagent in conjunction with heat. For example and without wishing to be bound by theory, in some embodiments, Tween 20 is a lysis reagent and the presence of Tween 20 and heat may inactivate one or more amplification reagents (e.g., a polymerase) or degrade a target nucleic acid (e.g., by lysing a cell, e.g., releasing a nuclease).

In some embodiments, a method for detecting a target nucleic acid comprises applying heat to a biological sample or sample-containing fluid to resolve a mucous matrix and applying heat to a biological sample or sample-containing fluid to lyse the sample and/or amplify a target nucleic acid. In some embodiments, application of heat to resolve a mucous matrix is a separate step from application of heat to lyse the sample and/or amplify a target nucleic acid; in other words, application of heat to resolve a mucous matrix begins and ends prior to the beginning and ending of application of heat to lyse the sample and/or amplify a target nucleic acid. In other embodiments, application of heat to resolve a mucous matrix overlaps with application of heat to lyse the sample and/or amplify a target nucleic acid. For example, in some embodiments, a method comprises applying heat to a biological sample or sample-containing fluid prior to resolve a mucous matrix prior to lysing a cell of the sample and/or prior to amplifying the sample, and continuing to apply heat upon lysing a cell of the sample (e.g., continuing to apply heat after contacting the sample-containing fluid with one or more lysis reagents) and/or continuing to apply heat during amplification of a target nucleic acid (e.g., continuing to apply heat after contacting the sample with one or more amplification reagents).

In some embodiments, heat is applied to a biological sample after contacting the biological sample with a diluent. In some embodiments, the diluent is water (i.e., consists of water). In some embodiments, a method for detecting a target nucleic acid comprises applying heat to a sample-containing fluid, wherein the sample-containing fluid is produced by combining a biological sample with water.

In some embodiments, applying heat to a biological sample or sample-containing fluid to resolve a mucous matrix comprises establishing and/or maintaining a first temperature in the biological sample or sample-containing fluid and applying heat to a biological sample or sample-containing fluid to lyse the sample and/or amplify a target nucleic acid comprises establishing and/or maintaining a second temperature in the biological sample or sample-containing fluid. In some embodiments, the first temperature and the second temperature are the same. In some embodiments, the first temperature and the second temperature are different. In some embodiments, applying heat to a biological sample or sample-containing fluid to resolve a mucous matrix comprises establishing and/or maintaining a first temperature in the biological sample or sample-containing fluid, applying heat to a biological sample or sample-containing fluid to lyse the sample comprises establishing and/or maintaining a second temperature in the biological sample or sample-containing fluid, and applying heat to a biological sample or sample-containing fluid to amplify a target nucleic acid comprises establishing and/or maintaining a third temperature in the biological sample or sample-containing fluid. In some embodiments, the first temperature, the second temperature, and the third temperature are the same. In some embodiments, the first temperature and the second temperature are the same and the third temperature is different from the first and second. In some embodiments, the first temperature and the third temperature are the same and the second temperature is different from the first and third. In some embodiments, the second temperature and the third temperature are the same and the first temperature is different from the second and third.

In some embodiments, a method comprises heating a sample to above about 55° C. (e.g., to about 60° C., about 65° C., about 70° C., about 75° C., about 80° C., about 85° C., about 90° C., or about 95° C.) to resolve a mucous matrix. In some embodiments, heating a sample to above about 55° C. (e.g., to about 60° C., about 65° C., about 70° C., about 75° C., about 80° C., about 85° C., about 90° C., or about 95° C.) resolves a mucous matrix and lyses the sample. In some embodiments, a method comprises heating a sample to about room temperature (e.g., 20° C.-25° C.) or above about 37° C. (e.g., to about 37° C., about 40° C., about 50° C., about 60° C., about 65° C., about 70° C., about 80° C., or about 90° C.) to lyse the sample. In some embodiments, a method comprises heating a sample to between about 60° C. and about 65° C. for amplification of a target nucleic acid (e.g., to about 60° C., about 61° C., about 62° C., about 63° C., about 64° C., or about 65° C.). For example, in some embodiments, a method comprises heating a sample to above about 55° C. (e.g., to about 60° C., about 65° C., about 70° C., about 75° C., about 80° C., about 85° C., about 90° C., or about 95° C.) to resolve a mucous matrix, to about room temperature (e.g., 20° C.-25° C.) or above about 37° C. (e.g., to about 37° C., about 40° C., about 50° C., about 60° C., about 65° C., about 70° C., about 80° C., or about 90° C.) to lyse the sample, and to between about 60° C. and about 65° C. for amplification of a target nucleic acid. As a further example, in some embodiments, a method comprises heating a sample to above about 55° C. (e.g., to about 60° C., about 65° C., about 70° C., about 75° C., about 80° C., about 85° C., about 90° C., or about 95° C.) to resolve a mucous matrix and lyse a sample and to between about 60° C. and about 65° C. for amplification of a target nucleic acid.

In some embodiments, a method for detecting a target nucleic acid comprising applying heat to a biological sample or sample-containing fluid prior to amplifying the sample, prior to lysing a cell of the sample, prior to applying the sample-containing fluid to a rapid testing device, and/or prior to adding a reagent to the sample (e.g., a matrix resolving agent, a lysis reagent, and/or an amplification reagent) has a higher detection rate for the target nucleic acid than an otherwise similar method that does not apply heat to the biological sample or sample-containing fluid with similar timing. In some embodiments, a method for detecting a target nucleic acid comprising applying heat to a biological sample or sample-containing fluid prior to amplifying the sample, prior to lysing a cell of the sample, prior to applying the sample-containing fluid to a rapid testing device, and/or prior to adding a reagent to the sample (e.g., a matrix resolving agent, a lysis reagent, and/or an amplification reagent) has a lower false negative rate for the target nucleic acid than an otherwise similar method that does not apply heat to the biological sample or sample-containing fluid with similar timing. In some embodiments, the method has a detection rate at least 1, 2, 3, 4, 5, 6, 7, 8, 9, 10, 12, 15, 17, 20, 22, 25, 27, 30, 32, 35, 37, 40, 45, 50, 60, 70, 80, 90, 95, 99, or 100% higher for the target nucleic acid than an otherwise similar method that does not apply heat to the biological sample or sample-containing fluid with similar timing. In some embodiments, the method has a detection rate at least 1-100, 1-80, 1-60, 1-50, 1-45, 1-40, 1-35, 1-30, 1-25, 1-20, 1-15, 1-10, 1-5, 5-100, 5-80, 5-60, 5-50, 5-45, 5-40, 5-35, 5-30, 5-25, 5-20, 5-15, 5-10, 10-100, 10-80, 10-60, 10-50, 10-45, 10-40, 10-35, 10-30, 10-25, 10-20, 10-15, 15-100, 15-80, 15-60, 15-50, 15-45, 15-40, 15-35, 15-30, 15-25, 15-20, 20-100, 20-80, 20-60, 20-50, 20-45, 20-40, 20-35, 20-30, 20-25, 25-100, 25-80, 25-60, 25-50, 25-45, 25-40, 25-35, 25-30, 30-100, 30-80, 30-60, 30-50, 30-45, 30-40, 30-35, 35-100, 35-80, 35-60, 35-50, 35-45, 35-40, 40-100, 40-80, 40-60, 40-50, 40-45, 45-100, 45-80, 45-60, 45-50, 50-100, 50-80, 50-60, 60-100, 60-80, or 80-100% higher for the target nucleic acid than a method that does not apply heat to the biological sample or sample-containing fluid with similar timing. In some embodiments, the method has a false negative rate at least 1, 2, 3, 4, 5, 6, 7, 8, 9, 10, 12, 15, 17, 20, 22, 25, 27, 30, 32, 35, 37, 40, 45, 50, 60, 70, 80, 90, 95, 99, or 100% lower for the target nucleic acid than an otherwise similar method that does not apply heat to the biological sample or sample-containing fluid with similar timing. In some embodiments, the method has a false negative at least 1-100, 1-80, 1-60, 1-50, 1-45, 1-40, 1-35, 1-30, 1-25, 1-20, 1-15, 1-10, 1-5, 5-100, 5-80, 5-60, 5-50, 5-45, 5-40, 5-35, 5-30, 5-25, 5-20, 5-15, 5-10, 10-100, 10-80, 10-60, 10-50, 10-45, 10-40, 10-35, 10-30, 10-25, 10-20, 10-15, 15-100, 15-80, 15-60, 15-50, 15-45, 15-40, 15-35, 15-30, 15-25, 15-20, 20-100, 20-80, 20-60, 20-50, 20-45, 20-40, 20-35, 20-30, 20-25, 25-100, 25-80, 25-60, 25-50, 25-45, 25-40, 25-35, 25-30, 30-100, 30-80, 30-60, 30-50, 30-45, 30-40, 30-35, 35-100, 35-80, 35-60, 35-50, 35-45, 35-40, 40-100, 40-80, 40-60, 40-50, 40-45, 45-100, 45-80, 45-60, 45-50, 50-100, 50-80, 50-60, 60-100, 60-80, or 80-100% lower for the target nucleic acid than a method that does not apply heat to the biological sample or sample-containing fluid with similar timing.

In some embodiments, a method of the disclosure comprises: (a) applying heat to a biological sample or sample-containing fluid prior to amplifying the sample, prior to lysing a cell of the sample, prior to applying the sample-containing fluid to a rapid testing device, and/or prior to adding a reagent to the sample (e.g., a matrix resolving agent, a lysis reagent, and/or an amplification reagent), and (b) combining a biological sample with a diluent and/or a matrix resolving agent (e.g., a diluent comprising a matrix resolving agent). Without wishing to be bound by theory, it is thought that the effects of applying heat to a biological sample comprising a mucous matrix with the timing described herein and of combining a biological sample comprising a mucous matrix with a diluent and/or a matrix resolving agent (e.g., a diluent comprising a matrix resolving agent) are complementary. In some embodiments, the beneficial effects of applying heat to a biological sample comprising a mucous matrix with the timing described herein and of combining a biological sample comprising a mucous matrix with a diluent and/or a matrix resolving agent (e.g., a diluent comprising a matrix resolving agent) are additive. In some embodiments, the improvement to a physico-chemical property or to a downstream process, e.g., a lysis step or an amplification step, obtained from applying heat to a biological sample comprising a mucous matrix with the timing described herein and of combining a biological sample comprising a mucous matrix with a diluent and/or a matrix resolving agent (e.g., a diluent comprising a matrix resolving agent) is greater than the corresponding improvement obtained from applying heat with the described timing or using a diluent and/or a matrix resolving agent alone. In some embodiments, a synergistic improvement to a physico-chemical property or to a downstream process, e.g., a lysis step or an amplification step, is obtained from applying heat to a biological sample comprising a mucous matrix with the timing described herein and of combining a biological sample comprising a mucous matrix with a diluent and/or a matrix resolving agent (e.g., a diluent comprising a matrix resolving agent), e.g., an improvement greater than the expected additive improvement from applying heat with the timing described or use of a diluent and/or a matrix resolving agent alone.

Biological Samples Aspects of the disclosure relate to methods for detecting one or more target nucleotides in a biological sample. In some embodiments, a biological sample is obtained from a subject (e.g., a human subject, an animal subject). In some embodiments, a biological sample comprises a mucous matrix. Exemplary biological samples include one or more of mucus, saliva, sputum, or cell scrapings (e.g., a scraping from the mouth or interior cheek). In some embodiments, a biological sample comprises saliva and/or mucus. In some embodiments, a biological sample can be collected from a subject who is also the user of a method or device described herein. For example, a subject may collect their own biological sample using a method or device described herein.

In some embodiments, the biological sample comprises a mucous matrix. In some embodiments, the mucous matrix comprises mucus. Many exterior exposed surfaces of the human body comprise mucous matrix containing secretions that protect epithelial surfaces from desiccation, particulates, pathogens, and toxicants. For example, the airways of the respiratory tract, including the nasal and esophageal passages, are coated in airway surface liquid containing mucous matrices.

In some embodiments, the biological sample comprises a nasal secretion. In certain instances, for example, the sample is an anterior nares specimen. An anterior nares specimen may be collected from a subject by inserting a swab element of a sample-collecting component into one or both nostrils of the subject for a period of time. In some embodiments, the period of time is at least 5 seconds, at least 10 seconds, at least 20 seconds, or at least 30 seconds. In some embodiments, the period of time is 30 seconds or less, 20 seconds or less, 10 seconds or less, or 5 seconds or less. In some embodiments, the period of time is in a range from 5 seconds to 10 seconds, 5 seconds to 20 seconds, 5 seconds to 30 seconds, 10 seconds to 20 seconds, or 10 seconds to 30 seconds. In some embodiments, the biological sample comprises a cell scraping. In certain embodiments, the cell scraping is collected from the mouth or interior cheek. The cell scraping may be collected using a brush or scraping device formulated for this purpose. The sample may be self-collected by the subject or may be collected by another individual (e.g., a family member, a friend, a coworker, a health care professional) using a sample-collecting component described herein.

In some embodiments, the sample comprises an oral secretion (e.g., saliva). In certain cases, the volume of saliva in the sample is at least 1 mL, at least 1.5 mL, at least 2 mL, at least 2.5 mL, at least 3 mL, at least 3.5 mL, or at least 4 mL. In some embodiments, the volume of saliva in the sample is in a range from 1 mL to 2 mL, 1 mL to 3 mL, 1 mL to 4 mL, or 2 mL to 4 mL. Saliva has been found to have a mean concentration of SARS-Cov-2 RNA of 5 fM (Kai-Wang To et al., 2020)— an amount that is detectable by any one of the methods described herein. In some embodiments, methods described herein are capable of detecting a concentration of a target nucleic acid (e.g., SARS-Cov-2 RNA) in a biological sample that is less than 5 fM.

In some embodiments, a biological sample (e.g., nasal secretion or saliva sample) is deposited directly into a reaction tube. In some embodiments, the concentration of a target nucleic acid molecule (e.g., SARS-CoV-2 RNA) in the biological sample is at least 5 aM, at least 10 aM, at least 15 aM, at least 20 aM, at least 25 aM, at least 30 aM, at least 35 aM, at least 40 aM, at least 50 aM, at least 75 aM, at least 100 aM, at least 150 aM, at least 200 aM, at least 300 aM, at least 400 aM, at least 500 aM, at least 600 aM, at least 700 aM, at least 800 aM, at least 900 aM, at least 1 fM, at least 5 fM, at least 10 fM, at least 15 fM, at least 20 fM, at least 25 fM, at least 30 fM, at least 35 fM, at least 40 fM, at least 50 fM, at least 75 fM, at least 100 fM, at least 150 fM, at least 200 fM, at least 300 fM, at least 400 fM, at least 500 fM, at least 600 fM, at least 700 fM, at least 800 fM, at least 900 fM, at least 1 pM, at least 5 pM, or at least 10 pM. In some embodiments, the concentration of a target nucleic acid molecule (e.g., SARS-CoV-2 RNA) in the biological sample is 10 pM or less, 5 pM or less, 1 pM or less, 500 fM or less, 100 fM or less, 50 fM or less, 10 fM or less, 1 fM or less, 500 aM or less, 100 aM or less, 50 aM or less 10 aM or less, or 5 aM or less. In some embodiments, the concentration of a target nucleic acid molecule (e.g., SARS-CoV-2 RNA) in the biological sample is in a range from 5 aM to 50 aM, 5 aM to 100 aM, 5 aM to 500 aM, 5 aM to 1 fM, 5 aM to 10 fM, 5 aM to 50 fM, 5 aM to 100 fM, 5 aM to 500 fM, 5 aM to 1 pM, 5 aM to 10 pM, 10 aM to 50 aM, 10 aM to 100 aM, 10 aM to 500 aM, 10 aM to 1 fM, 10 aM to 10 fM, 10 aM to 50 fM, 10 aM to 100 fM, 10 aM to 500 fM, 10 aM to 1 pM, 10 aM to 10 pM, 100 aM to 500 aM, 100 aM to 1 fM, 100 aM to 10 fM, 100 aM to 50 fM, 100 aM to 100 fM, 100 aM to 500 fM, 100 aM to 1 pM, 100 aM to 10 pM, 1 fM to 10 fM, 1 fM to 50 fM, 1 fM to 100 fM, 1 fM to 500 fM, 1 fM to 1 pM, 1 fM to 10 pM, 5 fM to 10 fM, 5 fM to 50 fM, 5 fM to 100 fM, 5 fM to 500 fM, 5 fM to 1 pM, 5 fM to 10 pM, 10 fM to 100 fM, 10 fM to 500 fM, 10 fM to 1 pM, 10 fM to 10 pM, 100 fM to 500 fM, 100 fM to 1 pM, 100 fM to 10 pM, or 1 pM to 10 pM.

The biological sample, in some embodiments, is collected from a subject who is suspected of having the disease(s) the test screens for, such as a coronavirus (e.g., COVID-19) and/or influenza (e.g., influenza type A or influenza type B). Other indications, as described herein, are also envisioned. In some embodiments, the subject is a human. Subjects may be asymptomatic, or may present with one or more symptoms of the disease(s). Symptoms of coronaviruses (e.g., COVID-19) include, but are not limited to, fever, cough (e.g., dry cough), generalized fatigue, sore throat, headache, loss of taste or smell, runny nose, nasal congestion, muscle aches, and difficulty breathing (shortness of breath). Symptoms of influenza include, but are not limited to, fever, chills, muscle aches, cough, congestion, runny nose, headaches, and generalized fatigue. In some embodiments, the subject is asymptomatic, but has had contact within the past 14 days with a person that has tested positive for the virus.

A subject may be any mammal, for example a human, non-human primate (e.g., monkey, chimpanzee, ape, etc.), dog, cat, pig, horse, hamster, guinea pig, rat, mouse, etc. In some embodiments, a subject is a human. In some embodiments, a subject is an adult human (e.g., a human older than 16 years of age, 18 years of age, etc.). In some embodiments, a subject is a child (e.g., a pediatric subject), for example a subject that is less than 18 years of age, 16 years of age, etc. In some embodiments, a subject is an infant, for example a subject less than one year of age.

### Nucleic Acids

The disclosure relates, in some aspects, to methods and systems for detecting nucleic acids and nucleic acid sequences. A “nucleic acid” sequence refers to a DNA or RNA (or a sequence encoded by DNA or RNA). In some embodiments, a nucleic acid is isolated. As used herein, the term “isolated” means artificially produced. As used herein, with respect to nucleic acids, the term “isolated” means: (i) amplified in vitro by, for example, polymerase chain reaction (PCR); (ii) recombinantly produced by cloning; (iii) purified, as by cleavage and gel separation; or (iv) synthesized by, for example, chemical synthesis. An isolated nucleic acid is one which can be manipulated by recombinant DNA techniques well known in the art. An isolated nucleic acid may be substantially purified, but need not be. For example, a nucleic acid that is isolated within a cloning or expression vector is not pure in that it may comprise only a tiny percentage of the material in the cell in which it resides. Such a nucleic acid is isolated, however, as the term is used herein because it is readily manipulable by standard techniques known to those of ordinary skill in the art.

In some embodiments, a nucleic acid or isolated nucleic acid is a referred to as a “polynucleotide” or “oligonucleotide.” The terms “polynucleotide” and “oligonucleotide” refer to nucleic acids comprising two or more units (e.g., nucleotides) connected by a phosphate-based backbone (e.g., a sugar-phosphate backbone), for example genomic DNA (gDNA), complementary DNA (cDNA), RNA (e.g., mRNA, shRNA, dsRNA, miRNA, tRNA, etc.), synthetic nucleic acids and synthetic nucleic acid analogs. Polynucleotides (or oligonucleotides) may include natural or non-natural bases, or combinations thereof and natural or non-natural backbone linkages, such as phosphorothioate linkages, peptide nucleic acids (PNA), 2′-0-methyl-RNA, or combinations thereof.

The length of a polynucleotide (or each strand of a double stranded or duplex molecule) may vary. In some embodiments, a polynucleotide ranges from about 2 to 10, 2 to 20, 2 to 30, 2 to 40, 2 to 50, 2 to 75, 2 to 100, 2 to 150, 2 to 200, 2 to 300, 2 to 400, 2 to 500, 2 to 1000, 2 to 2000, 2 to 5000, 2 to 10,000, 2 to 50,000, 2 to 500,000, or 2 to 1,000,000 nucleotides in length. In some embodiments, a polynucleotide is more than 1,000,000 nucleotides in length (e.g., longer than a megabase). A nucleic acid (e.g., a polynucleotide) may be single stranded or double stranded.

In some embodiments, a nucleic acid (e.g., polynucleotide) is a primer. As used herein, a “primer” refers to a polynucleotide that is capable of selectively binding (e.g., hybridizing or annealing) to a nucleic acid template and allows the synthesis of a sequence complementary to the corresponding polynucleotide template. A nucleic acid template may be a target nucleic acid or control nucleic acid. In some embodiments, a nucleic acid template comprises a region of complementarity with one or more primers. Generally, a primer ranges in size from about 10 to 100 nucleotides, and functions as a point of initiation for template-directed, polymerase mediated synthesis of a polynucleotide complementary to the template. In some embodiments, a primer is specific for a target nucleic acid.

A nucleic acid may be unmodified or modified. A modified nucleotide may comprise one or more modified nucleic acid bases and/or a modified nucleic acid backbone. In some embodiments, a modified nucleic acid comprises one or more nucleotide analogs.

In some embodiments, a nucleic acid is modified by conjugation to one or more biological or chemical moieties. Examples of moieties used for modifying nucleic acids include fluorophores, radioisotopes, chromophores, purification tags (e.g., polyHis, FLAG, biotin, etc.), barcoding molecules, haptens (e.g., FITC, digoxigenin (DIG), fluorescein, bovine serum albumin (BSA), dinitrophenyl, oxazole, pyrazole, thiazole, nitroaryl, benzofuran, triperpene, urea, thiourea, rotenoid, coumarin, etc.), extension blocking groups, and combinations thereof. In some embodiments, a nucleic acid (e.g., a primer) comprises one or more modifications (e.g., moieties described herein). In some embodiments, a modified nucleic acid comprises an extension blocking group or a hapten.

### Target Nucleic Acids

Methods and devices described by the disclosure may be used, in some embodiments, to detect the presence or absence of any target nucleic acid sequence (e.g., from any pathogen of interest). Target nucleic acid sequences may be associated with a variety of diseases or disorders, as described below. In some embodiments, the diagnostic devices, systems, and methods are used to diagnose at least one disease or disorder caused by a pathogen. In certain instances, the diagnostic devices, systems, and methods are configured to detect a nucleic acid encoding a protein (e.g., a nucleocapsid protein) of SARS-CoV-2, which is the virus that causes COVID-19. In some embodiments, the diagnostic devices, systems, and methods are configured to identify particular strains of a pathogen (e.g., a virus). In certain embodiments, a diagnostic device comprises a lateral flow assay strip comprising a first test line configured to detect a nucleic acid sequence of SARS-CoV-2 and a second test line configured to detect a nucleic acid sequence of a SARS-CoV-2 virus having a D614G mutation (i.e., a mutation of the 614th amino acid from aspartic acid (D) to glycine (G)) in its spike protein. In some embodiments, one or more target nucleic acid sequences are associated with a single-nucleotide polymorphism (SNP). In certain cases, diagnostic devices, systems, and methods described herein may be used for rapid genotyping to detect the presence or absence of a SNP, which may affect medical treatment.

In some embodiments, the diagnostic devices, systems, and methods are configured to diagnose two or more diseases or disorders. In certain cases, for example, a diagnostic device comprises a lateral flow assay strip comprising a first test line configured to detect a nucleic acid sequence of SARS-CoV-2 and a second test line configured to detect a nucleic acid sequence of an influenza virus (e.g., an influenza A virus or an influenza B virus). In some embodiments, a diagnostic device comprises a lateral flow assay strip comprising a first test line configured to detect a nucleic acid sequence of a virus and a second test line configured to detect a nucleic acid sequence of a bacterium. In some embodiments, a diagnostic device comprises a lateral flow assay strip comprising three or more test lines (e.g., test lines configured to detect SARS-CoV-2, SARS-CoV-2 D614G, an influenza type A virus, and/or an influenza type B virus). In some embodiments, a diagnostic device comprises a lateral flow assay strip comprising four or more test lines (e.g., test lines configured to detect SARS-CoV-2, SARS-CoV-2 D614G, an influenza type A virus, and/or an influenza type B virus).

In some embodiments, a diagnostic device, system, or method is configured to detect at least 1, at least 2, at least 3, at least 4, at least 5, at least 6, at least 7, at least 8, at least 9, or at least 10 target nucleic acid sequences. Each target nucleic acid sequence may independently be a nucleic acid of a pathogen (e.g., a viral, bacterial, fungal, protozoan, or parasitic pathogen) and/or a cancer cell.

In some embodiments, the diagnostic devices, systems, and methods are configured to detect a target nucleic acid sequence of a viral pathogen. Non-limiting examples of viral pathogens include coronaviruses, influenza viruses, rhinoviruses, parainfluenza viruses (e.g., parainfluenza 1-4), enteroviruses, adenoviruses, respiratory syncytial viruses, and metapneumoviruses. In certain embodiments, the viral pathogen is SARS-CoV-2 and/or SARS-CoV-2 D614G. In certain embodiments, the viral pathogen is an influenza virus. The influenza virus may be an influenza A virus (e.g., H1N1, H3N2) or an influenza B virus.

In some embodiments, the diagnostic devices, systems, and methods are configured to detect a target nucleic acid sequence of a bacterium (e.g., a bacterial pathogen). In some embodiments, the diagnostic devices, systems, and methods are configured to detect a target nucleic acid sequence of a fungus (e.g., a fungal pathogen). In some embodiments, the diagnostic devices, systems, and methods are configured to detect a target nucleic acid sequence of one or more protozoa (e.g., a protozoan pathogen)

In some embodiments, the diagnostic devices, systems, and methods are configured to detect a target nucleic acid sequence of a cancer cell. Cancer cells have unique mutations found in tumor cells and absent in normal cells. In some embodiments, the diagnostic devices, systems, and methods are configured to examine a subject's predisposition to certain types of cancer based on specific genetic mutations. In some embodiments, the diagnostic devices, systems, and methods are configured to detect a target nucleic acid sequence associated with a genetic disorder. In some embodiments, the diagnostic devices, systems, and methods are configured to detect a target nucleic acid sequence of an animal pathogen.

### Control Nucleic Acid Sequences

In some embodiments, methods and systems described herein comprise (or use) primers designed to amplify a human or animal nucleic acid that is not associated with a target nucleic acid from a pathogen, a cancer cell, or a contaminant. In some such embodiments, the human or animal nucleic acid may act as a control and is referred to as a “control nucleic acid”. For example, successful amplification and detection of a control nucleic acid may indicate that a sample was properly collected, and the diagnostic test was properly run (e.g., an amplification reaction was successful).

### Lysis of Sample

A biological sample may be lysed prior to (or while) performing an amplification reaction (e.g., an isothermal amplification reaction, such as RT-LAMP). In some embodiments, lysis of a sample is performed after combining the sample with a matrix resolving agent or a diluent (e.g., comprising a matrix resolving agent). In some embodiments, lysis of a sample is performed in conjunction with combining the sample with a matrix resolving agent or a diluent (e.g., comprising a matrix resolving agent). In some embodiments, one or more lysis reagents are provided in a solid form with one or more solid matrix resolving agents, e.g., lyophilized together. In some embodiments, a matrix resolving agent is provided to one or more lysis reagents in aqueous (e.g., rehydrated) form.

In some embodiments, lysis of a biological sample is performed by chemical lysis (e.g., exposing a sample to one or more lysis reagents) and/or thermal lysis (e.g., heating a sample). Chemical lysis may be performed by one or more lysis reagents. In some embodiments, the one or more lysis reagents comprise one or more enzymes. Non-limiting examples of suitable enzymes include lysozyme, lysostaphin, zymolase, cellulase, protease, and glycanase.

In some embodiments, the one or more lysis reagents comprise one or more detergents. Non-limiting examples of suitable detergents include sodium dodecyl sulphate (SDS), Tween (e.g., Tween 20, Tween 80), 3-[(3-cholamidopropyl)dimethylammonio]-1-propanesulfonate (CHAPS), 3-[(3-cholamidopropyl)dimethylammonio]-2-hydroxy-1-propanesulfonate (CHAPSO), Triton X-100, and NP-40. In some embodiments, an amplification buffer described herein comprises one or more detergents. In some embodiments, the one or more detergents comprises Tween.

In some embodiments, combining a biological sample or a sample-containing fluid with one or more lysis reagents produces a biological sample or sample-containing fluid with a volume of at least about 100 μl, at least about 200 μl, at least about 300 μl, at least about 400 μl, at least about 500 μl, at least about 600 μl, at least about 700 μl, at least about 800 μl, at least about 900 μl, or at least about 1000 μl. In some embodiments, combining a biological sample or a sample-containing fluid with one or more lysis reagents produces a biological sample or sample-containing fluid with a volume of 100-1000 μl, 200-1000 μl, 300-1000 μl, 400-1000 μl, 500-1000 μl, 600-1000 μl, 700-1000 μl, 800-1000 μl, 900-1000 μl, 100-900 μl, 200-900 μl, 300-900 μl, 400-900 μl, 500-900 μl, 600-900 μl, 700-900 μl, 800-900 μl, 100-800 μl, 200-800 μl, 300-800 μl, 400-800 μl, 500-800 μl, 600-800 μl, 700-800 μl, 100-700 μl, 200-700 μl, 300-700 μl, 400-700 μl, 500-700 μl, 600-700 μl, 100-600 μl, 200-600 μl, 300-600 μl, 400-600 μl, 500-600 μl, 100-500 μl, 200-500 μl, 300-500 μl, 400-500 μl, 100-400 μl, 200-400 μl, 300-400 μl, 100-300 μl, 200-300 μl, or 100-200 μl.

In some cases, at least one of the one or more lysis reagents is in solid form (e.g., lyophilized, dried, crystallized, air jetted). In some cases, all of the one or more lysis reagents are in solid form (e.g., lyophilized, dried, crystallized, air jetted). In certain embodiments, one or more lysis reagents are in the form of a lysis pellet or tablet. The lysis pellet or tablet may comprise any lysis reagent described herein. In certain embodiments, the lysis pellet or tablet may comprise one or more additional reagents (e.g., reagents to reduce or eliminate cross contamination). In a particular, non-limiting embodiment, a lysis pellet or tablet comprises Thermolabile Uracil-DNA Glycosylase (UDG) (e.g., at a concentration of about 0.02 U/μL) and murine RNAse inhibitor (e.g., at a concentration of about 1 U/μL).

In some embodiments, the one or more lysis reagents are active at approximately room temperature (e.g., 20° C.-25° C.). In some embodiments, the one or more lysis reagents are active at elevated temperatures (e.g., at least 37° C., at least 40° C., at least 50° C., at least 60° C., at least 65° C., at least 70° C., at least 80° C., at least 90° C.). In some embodiments, cell lysis is accomplished by applying heat to a sample (thermal lysis). In certain instances, thermal lysis is performed by applying a lysis heating protocol comprising heating the sample at one or more temperatures for one or more time periods using any heater described herein.

### Nucleic Acid Amplification

Following combining a biological sample with a matrix resolving agent or diluent (e.g., comprising a matrix resolving agent), or a lysis step, one or more target nucleic acids (e.g., a nucleic acid of a target pathogen) may be amplified. In some cases, a target pathogen may have RNA as its genetic material. In certain instances, for example, a target pathogen may be an RNA virus (e.g., a coronavirus, an influenza virus). In some such cases, the target pathogen's RNA may need to be reverse transcribed to DNA prior to amplification.

In some embodiments of the present technology, reverse transcription may be performed by exposing lysate to one or more reverse transcription reagents. In certain instances, the one or more reverse transcription reagents may comprise a reverse transcriptase, a DNA-dependent polymerase, and/or a ribonuclease (RNase). A reverse transcriptase generally may refer to an enzyme that transcribes RNA to complementary DNA (cDNA) by polymerizing deoxyribonucleotide triphosphates (dNTPs). An RNase generally may refer to an enzyme that catalyzes the degradation of RNA. In some cases, an RNase may be used to digest RNA from an RNA-DNA hybrid.

In some embodiments of the present technology, DNA may be amplified according to any nucleic acid amplification method known in the art. In some embodiments, the nucleic acid amplification method may be an isothermal amplification method. Isothermal amplification methods may include, but are not limited to, loop-mediated isothermal amplification (LAMP), recombinase polymerase amplification (RPA), nicking enzyme amplification reaction (NEAR), nucleic acid sequence-based amplification (NASBA), strand displacement amplification (SDA), helicase-dependent amplification (HDA), isothermal multiple displacement amplification (IMDA), rolling circle amplification (RCA), transcription mediated amplification (TMA), signal mediated amplification of RNA technology (SMART), single primer isothermal amplification (SPIA), circular helicase-dependent amplification (cHDA), and whole genome amplification (WGA). In one embodiment, the nucleic acid amplification method may be loop-mediated isothermal amplification (LAMP).

In some embodiments of the present technology, at least one of the one or more amplification reagents may be in solid form (e.g., lyophilized, dried, crystallized, air jetted, etc.). In some cases, all of the one or more amplification reagents may be in solid form (e.g., lyophilized, dried, crystallized, air jetted, etc.). In certain embodiments, one or more amplification reagents may be in the form of an amplification pellet, capsule, gelcap, or tablet. The amplification pellet, capsule, gelcap, or tablet may comprise any amplification reagent described herein.

In some embodiments, combining a biological sample or a sample-containing fluid with one or more amplification reagents produces a biological sample or sample-containing fluid with a volume of at least about 100 μl, at least about 200 μl, at least about 300 μl, at least about 400 μl, at least about 500 μl, at least about 600 μl, at least about 700 μl, at least about 800 μl, at least about 900 μl, at least about 1000 μl, at least about 1200 μl, at least about 1400 μl, at least about 1500 μl, at least about 1600 μl, at least about 1800 μl, or at least about 2000 μl. In some embodiments, combining a biological sample or a sample-containing fluid with one or more amplification reagents produces a biological sample or sample-containing fluid with a volume of 100-2000 μl, 200-2000 μl, 300-2000 μl, 400-2000 μl, 500-2000 μl, 600-2000 μl, 700-2000 μl, 800-2000 μl, 900-2000 μl, 1000-2000 μl, 1500-2000 μl, 100-1500 μl, 200-1500 μl, 300-1500 μl, 400-1500 μl, 500-1500 μl, 600-1500 μl, 700-1500 μl, 800-1500 μl, 900-1500 μl, 1000-1500 μl, 100-1000 μl, 200-1000 μl, 300-1000 μl, 400-1000 μl, 500-1000 μl, 600-1000 μl, 700-1000 μl, 800-1000 μl, 900-1000 μl, 100-900 μl, 200-900 μl, 300-900 μl, 400-900 μl, 500-900 μl, 600-900 μl, 700-900 μl, 800-900 μl, 100-800 μl, 200-800 μl, 300-800 μl, 400-800 μl, 500-800 μl, 600-800 μl, 700-800 μl, 100-700 μl, 200-700 μl, 300-700 μl, 400-700 μl, 500-700 μl, 600-700 μl, 100-600 μl, 200-600 μl, 300-600 μl, 400-600 μl, 500-600 μl, 100-500 μl, 200-500 μl, 300-500 μl, 400-500 μl, 100-400 μl, 200-400 μl, 300-400 μl, 100-300 μl, 200-300 μl, or 100-200 μl.

In some embodiments of the present technology, an isothermal amplification method described herein may comprise applying heat to a sample. In certain instances, an amplification method may comprise applying an amplification heating protocol comprising heating the sample at one or more temperatures for one or more time periods using any appropriate heater, such as any of the heaters described herein. In some embodiments of the present technology, a lysis heating protocol may comprise heating a sample at one or more additional temperatures for one or more additional time periods.

### LAMP

In some embodiments of the present technology, the nucleic acid amplification reagents may be LAMP reagents. LAMP refers to a method of amplifying a target nucleic acid through the creation of a series of stem-loop structures using a plurality of primers. Due to its use of multiple primers, LAMP may be highly specific for a target nucleic acid sequence.

In some embodiments of the present technology, the LAMP reagents may comprise four or more primers. In certain embodiments, the four or more primers may comprise a forward inner primer (FIP), a backward inner primer (BIP), a forward outer primer (F3), and a backward outer primer (B3). In some cases, the four or more primers may target at least six specific regions of a target gene. In some embodiments, the LAMP reagents may further comprise a forward loop primer (Loop F or LF) and a backward loop primer (Loop B or LB). In certain cases, the loop primers may target cyclic structures formed during amplification and may accelerate amplification.

Methods of designing LAMP primers are known in the art. In some cases, LAMP primers may be designed for each target nucleic acid a diagnostic device is configured to detect. For example, a diagnostic device configured to detect a first target nucleic acid (e.g., a nucleic acid of SARS-CoV-2) and a second target nucleic acid (e.g., a nucleic acid of an influenza virus) may comprise a first set of LAMP primers directed to the first target nucleic acid and a second set of LAMP primers directed to the second target nucleic acid. In some embodiments, the LAMP primers may be designed by alignment and identification of conserved sequences in a target pathogen (e.g., using Clustal X or a similar program) and then using a software program (e.g., PrimerExplorer). The specificity of different candidate primers may be confirmed using a BLAST search of the GenBank nucleotide database. Primers may be synthesized using any method known in the art.

In some embodiments of the present technology, the LAMP reagents may comprise deoxyribonucleotide triphosphates (“dNTPs”). In some embodiments of the present technology, the LAMP reagents may comprise magnesium sulfate (MgSO4). In some embodiments of the present technology, the LAMP reagents may comprise betaine.

### RPA

In some embodiments of the present technology, the nucleic acid amplification reagents may be RPA reagents. RPA generally refers to a method of amplifying a target nucleic acid using a recombinase, a single-stranded DNA binding protein, and a strand-displacing polymerase.

In some embodiments of the present technology, the RPA reagents may comprise a probe, a forward primer, and a reverse primer. The probe, forward primer, and reverse primer may be designed for each target nucleic acid a diagnostic device is configured to detect.

In some embodiments, the RPA reagents may comprise a reverse primer. In certain embodiments, the reverse primer may be at least 70%, at least 75%, at least 80%, at least 85%, at least 90%, at least 95%, at least 99%, or 100% identical to SEQ ID NO: 23. In some embodiments, the reverse primer may be at least 1 base pair, at least 2 base pairs, at least 3 base pairs, at least 4 base pairs, or at least 5 base pairs longer or shorter than SEQ ID NO: 23. In some embodiments, the reverse primer may comprise an antigenic tag.

In some embodiments of the present technology, the RPA reagents may further comprise a probe. In certain embodiments, the probe may be at least 70%, at least 75%, at least 80%, at least 85%, at least 90%, at least 95%, at least 99%, or 100% identical to SEQ ID NO: 24

In some embodiments of the present technology, the RPA reagents may comprise RPA primers designed to amplify a human or animal nucleic acid that may not be associated with a pathogen, a cancer cell, or a contaminant. In some such embodiments, the human or animal nucleic acid may act as a control. In some embodiments of the present technology, the control nucleic acid may be a nucleic acid sequence encoding human RNase P. In some embodiments, the RPA reagents may comprise primers (e.g., forward primers, reverse primers) and probes configured to detect a nucleic acid sequence encoding human RNase P.

In some embodiments of the present technology, the RPA reagents may comprise one or more recombinase enzymes. Non-limiting examples of suitable recombinase enzymes include T4 UvsX protein and T4 UvsY protein. In some embodiments of the present technology, the RPA reagents may comprise one or more single-stranded DNA binding proteins. A non-limiting example of a suitable single-stranded DNA binding protein is T4 gp32 protein. In some embodiments of the present technology, the RPA agents may comprise a DNA polymerase. A non-limiting example of a suitable DNA polymerase is Staphylococcus aureus DNA polymerase (Sau). In some embodiments of the present technology, the RPA agents may comprise an endonuclease. A non-limiting example of a suitable endonuclease is Endonuclease IV. In some embodiments of the present technology, the RPA reagents may comprise dNTPs (e.g., dATP, dGTP, dCTP, dTTP). In some embodiments of the present technology, the RPA reagents may comprise one or more additional components. Non-limiting examples of suitable components include DL-Dithiothreitol, phosphocreatine disodium hydrate, creatine kinase, and adenosine 5′-triphosphate disodium salt.

### Nicking Enzyme Amplification Reaction (NEAR)

In some embodiments of the present technology, amplification of one or more target nucleic acids may be accomplished through the use of a nicking enzyme amplification reaction (NEAR). NEAR generally refers to a method for amplifying a target nucleic acid using a nicking endonuclease and a strand displacing DNA polymerase. In some cases, NEAR may allow for amplification of very small amplicons.

In some embodiments of the present technology, the NEAR reagents may comprise a forward template and a reverse template. In certain embodiments, the forward template may comprises a nucleic acid sequence having a hybridization region at the 3′ end that is complementary to the 3′ end of a target antisense strand (e.g., an antisense sequence to the reverse-transcribed SARS-CoV-2 nucleocapsid sequence), a nicking enzyme binding site and a nicking site upstream of the hybridization region, and a stabilizing region upstream of the nicking site. In certain embodiments, the first reverse template may comprises a nucleic acid sequence having a hybridization region at the 3′ end that is complementary to the 3′ end of a target gene sense strand (e.g., a SARS-CoV-2 nucleocapsid gene sense strand), a nicking enzyme binding site and a nicking site upstream of the hybridization region, and a stabilizing region upstream of the nicking site. Designs of templates suitable for NEAR methods disclosed herein are provided in, for example, U.S. Pat. Nos. 9,617,586 and 9,689,031, each of which is incorporated herein by reference.

In some embodiments of the present technology, the NEAR composition may further comprise a probe oligonucleotide. In certain embodiments, the probe may comprise a nucleotide sequence complementary to the target gene nucleotide sequence. In some instances, for example, the probe may be a SARS-CoV-2 specific probe. In some embodiments of the present technology, the probe may be conjugated to a detectable label. In some embodiments of the present technology, the NEAR reagents may comprise a DNA polymerase. In some embodiments, the NEAR reagents may comprise at least one nicking enzyme.

In some embodiments of the present technology, amplification may be performed under essentially isothermal conditions.

### Oligonucleotide Strand Displacement Probes

Aspects of the disclosure relate to the recognition that inclusion of certain oligonucleotide strand displacement (OSD) probes in isothermal amplification reactions (e.g., LAMP) allows for direct application of the resulting amplicon mixtures to immunoassay devices without any intervening fluid transfer or dilution steps. An “oligonucleotide strand displacement probe” generally refers to a modified polynucleotide primer comprising a region of complementarity with one or more target nucleotides that is capable of displacing pre-hybridized strands of target nucleic acid amplicons (e.g., amplicons generated by LAMP). Oligonucleotide stand-displacement probes are generally known, for example as described by Jiang et al., Angew Chem Int Ed Engl. 2014 Feb. 10; 53(7): 1845-1848; Phillips et al., Anal Chem. 2018 Jun. 5; 90(11): 6580-6586; and Bhadra et al. (2020) https://doi.org/10.1101/2020.04.13.039941.

### Detection

In some embodiments, amplified nucleic acids (i.e., amplicons of target nucleic acids or control nucleic acids) may be detected using any suitable methods. In some embodiments, one or more target nucleic acid sequences are detected using a nucleic acid detection device, e.g., that monitors amplification of the target nucleic acid in real time, e.g., by monitoring fluorescence. In some embodiments, one or more target nucleic acid sequences are detected using a lateral flow assay strip. In some embodiments, one or more target nucleic acid sequences are detected using a colorimetric assay.

Aspects of the disclosure relate to methods that result in detection of amplification of one or more target nucleic acids using a nucleic acid detection device. In some embodiments, a nucleic acid detection device is a device capable of detecting amplification of one or more target nucleic acids, e.g., in real time (i.e., as amplification is occurring). In some embodiments, a nucleic acid detection device is a device capable of amplifying one or more target nucleic acids and detecting amplification of the one or more target nucleic acids, e.g., in real time (i.e., as amplification is occurring). In some embodiments, a nucleic acid detection device detects amplification by monitoring fluorescence, e.g., a change in fluorescence associated with a nucleic acid binding dye or a change in fluorescence associated with incorporation of a nucleotide comprising a fluorescent moiety. Any amplification method known in the art can be used with a nucleic acid detection device of the disclosure. In some embodiments, a nucleic acid detection device is capable of amplifying a target nucleic acid using qLAMP (e.g., as described in the Examples).

Aspects of the disclosure relate to methods that result in detection of one or more signals on a lateral flow assay that are brighter (e.g., more intense, more easily visible to an unaided eye, etc.) than lateral flow assay signals produced using previous methods. In some embodiments, signals (e.g., colored bands) produced using isothermal amplification methods described herein are between 10% and 100% (e.g., about 10, 20, 30, 40, 50, 60, 70 80, 90, 95, 99, or 100%) brighter than lateral flow signals produced using previously available isothermal amplification methods.

In some embodiments, one or more target nucleic acid sequences are detected using a lateral flow assay strip (e.g., in a “chimney” detection component, a cartridge, a blister pack). In some embodiments, a fluidic sample (e.g., fluidic contents of a reaction tube, a reagent reservoir, and/or a blister pack chamber) is transported through the lateral flow assay strip via capillary action. In some embodiments, the fluidic sample may comprise labeled amplicons.

In some embodiments, the fluidic sample is introduced to a first sub-region (e.g., a sample pad) of the lateral flow assay strip. In certain embodiments, the fluidic sample subsequently flows through a second sub-region (e.g., a particle conjugate pad) comprising a plurality of labeled particles. In some cases, the particles comprise gold nanoparticles (e.g., colloidal gold nanoparticles). The particles may be labeled with any suitable label. Non-limiting examples of suitable labels include biotin, streptavidin, fluorescein isothiocyanate (FITC), fluorescein amidite (FAM), fluorescein, and digoxigenin (DIG). In some cases, as an amplicon-containing fluidic sample flows through the second sub-region (e.g., a particle conjugate pad), a labeled nanoparticle binds to a label of an amplicon, thereby forming a particle-amplicon conjugate.

In some embodiments, the fluidic sample (e.g., comprising a particle-amplicon conjugate) subsequently flows through a third sub-region (e.g., a test pad) comprising one or more test lines. In some embodiments, a first test line comprises a capture reagent (e.g., an immobilized antibody) configured to detect a first target nucleic acid. In some embodiments, a particle-amplicon conjugate may be captured by one or more capture reagents (e.g., immobilized antibodies), and an opaque marking may appear.

In certain embodiments, the lateral flow assay strip comprises one or more additional test lines. In some instances, each test line of the lateral flow assay strip is configured to detect a different target nucleic acid. In some instances, two or more test lines of the lateral flow assay strip are configured to detect the same target nucleic acid.

In certain embodiments, the third sub-region (e.g., the test pad) of the lateral flow assay strip further comprises one or more control lines.

In certain embodiments, the lateral flow assay strip comprises a fourth sub-region (e.g., a wicking area) to absorb fluid flowing through the lateral flow assay strip.

In some embodiments, the disclosure relates to rapid, self-administrable tests for detecting the presence of one or more target nucleic acids derived from one or more pathogens. A “self-administrable” test refers to a test in which all testing steps are performed by the subject of the test. In some embodiments, a “rapid test” refers to a test in which all testing steps (e.g., sample collection, lysis, isothermal amplification, detection, etc.) may be completed in less than 3 hours, less than 2 hours, or less than 1 hour. In some embodiments, a method of detecting a target nucleic acid described herein is a rapid test.

### Isothermal Amplification CRISPR-based Detection

In some embodiments of the present technology, a diagnostic method or device may use CRISPR/Cas detection techniques and/or a diagnostic system may comprise one or more reagents for performing CRISPR/Cas detection. CRISPR generally may refer to Clustered Regularly Interspaced Short Palindromic Repeats, and Cas generally may refer to a particular family of proteins. In some embodiments, the CRISPR/Cas detection platform or techniques may be combined with an isothermal amplification method to create a single step reaction (Joung et al., “Point-of-care testing for COVID-19 using SHERLOCK diagnostics,” 2020). For example, the amplification and CRISPR detection may be performed using reagents having compatible chemistries (e.g., reagents that do not interact detrimentally with one another and are sufficiently active to perform amplification and detection). In some embodiments, CRISPR/Cas detection may be combined with LAMP.

CRISPR/Cas detection platforms are known in the art. Examples of such platforms include SHERLOCK® and DETECTR® (see, e.g., Kellner et al., Nature Protocols, 2019, 14: 2986-3012; Broughton et al., Nature Biotechnology, 2020; Joung et al., 2020).

### Diagnostic Systems and Methods of Use

Diagnostic devices, systems, and methods described herein may be safely and easily operated or conducted by untrained individuals. Unlike prior art diagnostic tests, some embodiments described herein may not require knowledge of even basic laboratory techniques (e.g., pipetting). Similarly, some embodiments described herein may not require expensive laboratory equipment (e.g., thermocyclers). In some embodiments, reagents are contained within a reaction tube, a cartridge, and/or a blister pack, such that users are not exposed to any potentially harmful chemicals. Diagnostic devices, systems, and methods described herein are also highly sensitive and accurate. Through nucleic acid amplification, the diagnostic devices, systems, and methods are able to accurately detect the presence of extremely small amounts of a target nucleic acid.

As a result, the diagnostic devices, systems, and methods described herein may be useful in self-administered, at home, or non-medical facility contexts. In some embodiments, diagnostic devices described herein are relatively small and inexpensive. In some embodiments, any reagents contained within a diagnostic device or system described herein may be thermostabilized, and the diagnostic device or system may be shelf stable for a relatively long period of time.

The present disclosure provides diagnostic devices, systems, and methods for rapidly and in a home environment detecting one or more target nucleic acid sequences (e.g., a nucleic acid sequence of a pathogen, such as SARS-CoV-2 or an influenza virus). Such diagnostic devices, systems, and methods are also referred to herein as rapid test devices, systems, or methods, respectively. A diagnostic system, as described herein, may be self-administrable and comprise a sample-collecting component (e.g., a swab) and a diagnostic device. The diagnostic device may comprise a cartridge, a blister pack, and/or a “chimney” detection device, according to some embodiments. In some cases, the diagnostic device comprises a detection component (e.g., a lateral flow assay strip, a colorimetric assay), results of which are self-readable, or automatically read by a computer algorithm. In certain embodiments, the diagnostic device further comprises one or more reagents (e.g., matrix resolving agents, lysis reagents, nucleic acid amplification reagents, CRISPR/Cas detection reagents). In certain other embodiments, the diagnostic system separately includes one or more reaction tubes comprising the one or more reagents. The diagnostic device may also comprise an integrated heater, or the diagnostic system may comprise a separate heater. The isothermal amplification technique employed yields not only fast but very accurate results.

In some embodiments, a diagnostic device comprises a housing comprising a detection component comprising a “chimney.” In certain embodiments, the “chimney” detection component comprises a chimney configured to receive a reaction tube. In certain embodiments, the “chimney” detection component comprises a puncturing component configured to puncture the reaction tube. The puncturing component may comprise one or more blades, needles, or other elements capable of puncturing a reaction tube. In certain embodiments, the “chimney” detection component comprises a lateral flow assay strip. As described herein, the lateral flow assay strip may comprise one or more test lines configured to detect one or more target nucleic acid sequences. In some embodiments, the lateral flow assay strip further comprises one or more control lines.

In some embodiments, the “chimney” detection component comprises a chimney, a front panel, and a bottom panel comprising a lateral flow assay strip and a puncturing component. The chimney and the front panel may be integrally formed or may be separately formed. The chimney, the front panel, and the back panel may be formed from any suitable material(s). In some cases, for example, the chimney, the front panel, and/or the back panel comprise one or more thermoplastic materials and/or metals. In some embodiments, the chimney, the front panel, and/or the back panel may be manufactured by injection molding, an additive manufacturing technique (e.g., 3D printing), and/or a subtractive manufacturing technique (e.g., laser cutting).

In some embodiments, a diagnostic system comprises a sample-collecting component (e.g., a swab), a reaction tube comprising one or more reagents, and a “chimney” detection component. In some embodiments, the diagnostic system further comprises a heater, as described herein. In some embodiments, a reaction tube may be inserted into a “chimney” detection component following heating.

In some aspects, the disclosure is directed to a device or diagnostic system (e.g., a rapid test device) comprising a housing, wherein the housing accommodates one or more chambers. In some embodiments, the one or more chambers are in fluidic communication with one another. In some embodiments, the fluidic communication between a pair of chambers is toggleable (e.g., the passage of fluid from one chamber to another chamber may be permitted or prohibited). In some embodiments, a device or diagnostic system comprises a lysis chamber, e.g., accommodated in the housing, wherein the lysis chamber comprises at least one matrix resolving agent and at least one lysis reagent. In some embodiments, the lysis chamber is configured to receive a biological sample. In some embodiments, the lysis chamber is configured to receive a sample-containing fluid. In some embodiments, a device or diagnostic system comprises an amplification chamber, e.g., accommodated in the housing, comprising at least one amplification reagent. In some embodiments, the lysis chamber is in fluidic communication with the amplification chamber.

In some embodiments, a device or diagnostic system comprises a sample preparation chamber in addition to a lysis chamber, e.g., accommodated in the housing. In some embodiments, the sample preparation chamber comprises diluent. In some embodiments, the sample preparation chamber comprises a matrix resolving agent. In some embodiments, the sample preparation chamber is configured to combine the biological sample with a matrix resolving agent or diluent (e.g., comprising a matrix resolving agent) to produce a sample-containing fluid. In some embodiments, the sample preparation chamber is in fluidic communication with the lysis chamber. In some embodiments, the sample preparation chamber is configured to receive a biological sample (e.g., for a biological sample to be deposited therein). In some embodiments, a device or diagnostic system does not comprise a sample preparation chamber, but does comprise a lysis chamber, e.g., comprising diluent and/or a matrix resolving agent. In some embodiments, the lysis chamber is configured to combine a biological sample with a matrix resolving agent or diluent (e.g., comprising a matrix resolving agent) to produce a sample-containing fluid, e.g., in addition to combining a sample-containing fluid with one or more lysis reagents.

In some embodiments, the diagnostic system comprises a sample-collecting component. The sample-collecting component may be configured to collect a sample (e.g., a nasal secretion, an oral secretion, a cell scraping, blood, urine) from a subject (e.g., a human subject, an animal subject). In some embodiments, a sample collected by a sample-collecting component may be deposited in a device (e.g., a chamber of a device, e.g., a sample preparation chamber or lysis chamber) described herein or a reaction tube. In some embodiments, depositing a sample comprises agitating the sample-collecting component in the device (e.g., a chamber of a device, e.g., a sample preparation chamber or lysis chamber) or a reaction tube.

In some embodiments, the sample-collecting component comprises a swab element.

In some embodiments, the swab element of the sample-collecting component is proximal to a stem element (e.g., a handle, an applicator). In certain cases, the stem element facilitates collection of a sample with the swab element. In some instances, for example, the stem element facilitates insertion of the swab element into a nasal cavity (e.g., anterior nares) or an oral cavity of a subject. In some embodiments, the stem element comprises one or more markings and/or flanges. The markings and/or flanges may, in some instances, facilitate sample collection by indicating the appropriate depth of insertion (e.g., into a nasal cavity).

In some embodiments, the sample-collecting component is a breakable swab comprising a swab element and a stem element. In some embodiments, the stem element comprises a breakable section.

In some embodiments, the diagnostic system comprises one or more reagents (e.g., matrix resolving agents, lysis reagents, nucleic acid amplification reagents, or CRISPR/Cas detection reagents). In some instances, at least one reagent is contained within a diagnostic device (e.g., a cartridge, a blister pack, a “chimney” detection component) of a diagnostic system. In some instances, at least one reagent is provided separately from the diagnostic device. In certain cases, for example, the diagnostic system comprises one or more reaction tubes comprising the at least one reagent. In some embodiments, at least one of the one or more reagents is in liquid form (e.g., in solution). In some embodiments, at least one of the one or more reagents is in solid form (e.g., lyophilized, dried, crystallized, air jetted).

In some embodiments, the one or more lysis reagents comprise an RNase inhibitor (e.g., a murine RNase inhibitor). In some embodiments, the one or more lysis reagents comprise a protease (e.g., proteinase K).

In some embodiments, the one or more reagents comprise one or more reagents to reduce or eliminate potential carryover contamination from prior tests (e.g., prior tests conducted in the same area). In some embodiments, the one or more reagents comprise thermolabile uracil DNA glycosylase (UDG).

In certain embodiments, the one or more reagents comprise one or more reverse transcription reagents. In some cases, a target pathogen has RNA as its genetic material. In certain instances, for example, a target pathogen is an RNA virus (e.g., a coronavirus, an influenza virus). In some such cases, the target pathogen's RNA may need to be reverse transcribed to DNA prior to amplification.

In some embodiments, the one or more reagents comprise one or more nucleic acid amplification reagents. A nucleic acid amplification reagent generally refers to a reagent that facilitates a nucleic acid amplification method. In some embodiments, the nucleic acid amplification method is an isothermal nucleic acid amplification method. In certain embodiments, the one or more nucleic acid amplification reagents comprise LAMP reagents, RPA reagents, or NEAR reagents.

In some embodiments, the one or more reagents comprise one or more additives that enhance reagent stability (e.g., protein stability).

In some embodiments, the one or more reagents comprise one or more buffers. Non-limiting examples of suitable buffers include phosphate-buffered saline (PBS) and Tris. In some embodiments, the one or more buffers have a relatively neutral pH. In some embodiments, the one or more buffers have a pH in a range from 5.0 to 6.0, 5.0 to 7.0, 5.0 to 8.0, 5.0 to 9.0, 6.0 to 7.0, 6.0 to 8.0, 6.0 to 9.0, 7.0 to 8.0, 7.0 to 9.0, or 8.0 to 9.0. In some embodiments, the one or more reagents comprise one or more salts. Non-limiting examples of suitable salts include magnesium acetate tetrahydrate, potassium acetate, and potassium chloride.

In some embodiments, at least one reagent is not contained within a diagnostic device, and a diagnostic system comprises one or more reaction tubes. The one or more reaction tubes may contain any reagent(s) described above. In some embodiments, the one or more reaction tubes comprise at least one reagent in liquid form. In some embodiments, the one or more reaction tubes comprise at least one reagent in solid form.

The reaction tubes, in some embodiments, further comprise at least one cap. In some embodiments, the reaction tube comprises a partially removable cap (e.g., a hinged cap) or one or more wholly removable caps (e.g., one or more screw-top caps, one or more stoppers). In some embodiments, the one or more caps comprise reagents in solid form (e.g., lyophilized, dried, crystallized, air jetted reagents). In some embodiments, a diagnostic system or kit comprises the one or more caps separate from one or more reaction tubes. In some embodiments, a diagnostic system or kit comprises instructions for applying them to one or more reaction tubes as part of a method for using a diagnostic device of the system or kit. For example, in some embodiments a first cap comprises a first set of reagents (e.g., lysis reagents, diluent (e.g., comprising a matrix resolving agent), and/or a matrix resolving agent) and a second cap comprises a second set of reagents (e.g., nucleic acid amplification reagents).

In some embodiments, the fluidic contents of the reaction tube (e.g., diluent) comprise a reaction buffer. In certain instances, the reaction buffer comprises one or more buffers. Non-limiting examples of suitable buffers include phosphate-buffered saline (“PBS”) and Tris. In certain instances, the reaction buffer comprises one or more salts. Non-limiting examples of suitable salts include magnesium acetate tetrahydrate, potassium acetate, and potassium chloride.

In some embodiments, the reaction buffer comprises Tween (e.g., Tween 20, Tween 80). In some embodiments, the reaction buffer comprises an RNase inhibitor. In certain instances, Tween and/or an RNase inhibitor may facilitate cell lysis.

In a particular, non-limiting embodiment, the reaction buffer comprises 25 mM Tris buffer, 5% (w/v) poly(ethylene glycol) 35,000 kDa, 14 mM magnesium acetate tetrahydrate, 100 mM potassium acetate, and greater than 85% volume nuclease free water.

In some embodiments, the reaction buffer has a relatively neutral pH. In some embodiments, the reaction buffer has a pH in a range from 5.0 to 6.0, 5.0 to 7.0, 5.0 to 8.0, 5.0 to 9.0, 6.0 to 7.0, 6.0 to 8.0, 6.0 to 9.0, 7.0 to 8.0, 7.0 to 9.0, or 8.0 to 9.0.

In some embodiments, the diagnostic device comprises a colorimetric assay. In certain embodiments, the colorimetric assay comprises a cartridge comprising a central sample chamber in fluidic communication with a plurality of peripheral chambers (e.g., at least four peripheral chambers). In some embodiments, each peripheral chamber comprises isothermal nucleic acid amplification reagents comprising a unique set of primers (e.g., primers specific for one or more target nucleic acid sequences, primers specific for a positive test control, primers specific for a negative test control).

In operation, a sample may be deposited in the central sample chamber. In some cases, the sample may be combined with a reaction buffer in the central sample chamber. In certain cases, the central sample chamber may be heated to lyse cells within the sample. In some cases, the lysate may be directed to flow from the central sample chamber to the plurality of peripheral chambers comprising unique primers. In some cases, a colorimetric reaction may occur in each peripheral chamber, resulting in varying colors in the peripheral chambers. In some cases, the results within each peripheral chamber may be visible (e.g., through a clear film or other covering).

In some embodiments, a device may comprise one or more reagent reservoirs connected via one or more fluidic channels.

The diagnostic system, in some embodiments, comprises a heater. In certain embodiments, the heater is integrated with the diagnostic device. In some embodiments, the diagnostic system comprises a separate heater (i.e., a heater that is not integrated with other system components). In some embodiments, the heater is configured to receive a reaction tube.

In some embodiments, the heater is pre-programmed with one or more protocols. In some embodiments, for example, the heater is pre-programmed with a lysis heating protocol and/or an amplification heating protocol. A lysis heating protocol generally refers to a set of one or more temperatures and one or more time periods that facilitate lysis of the sample. An amplification heating protocol generally refers to a set of one or more temperatures and one or more time periods that facilitate nucleic acid amplification. In some embodiments, the heater comprises an auto-start mechanism that corresponds to the temperature profile needed for lysis and/or amplification. That is, a user may insert a reaction tube into the heater, and the heater may automatically run a lysis and/or amplification heating protocol. In some embodiments, the heater is controlled by a mobile application.

In some embodiments, the device may further comprise a pumping device, e.g., configured to facilitate fluid flow to and from one or more chambers and/or reagent reservoirs or to facilitate fluid flow to and from one or more reaction tubes.

In some embodiments, the device may be a disposable, single-use device. In some cases, the device may further comprise at least one container in which the device is stored before being used in a test procedure. In some cases, the at least one container may be sealed to prevent contamination of the device.

In some embodiments, the device, e.g., the housing, may be comprised of a window through which the lateral flow assay strip is visible.

In some embodiments, one or more components of a diagnostic system comprise a unique label. In some cases, this may advantageously allow multiple samples to be run in parallel. For example, one or more components of the diagnostic system (e.g., reaction tube cap, detection component) may be labeled with the same label. In some embodiments, a copy of the label is given to a tested subject, so that the subject may later receive the results using the unique label. In this way, multiple tests (one for each unique subject) may be run in parallel without mixing up the samples.

The disclosure is directed, in part, to methods of detecting a target nucleic acid, comprising combining a biological sample with a diluent to produce a sample-containing fluid. The disclosure is further directed, in part, to methods for detecting a target nucleic acid, comprising combining a biological sample with a transfer fluid and a matrix resolving agent to produce a sample-containing fluid. In some embodiments, the method comprises contacting a lateral flow assay strip having a first end and a second end with the sample-containing fluid. In some embodiments, the method comprises combining the biological sample or sample-containing fluid with one or more lysis reagents. In some embodiments, the method comprises amplifying the sample (e.g., a nucleic acid, e.g., a target nucleic, in the sample) by permitting the sample-containing fluid to interact with at least one amplification reagent, e.g., in an amplification chamber of a test device. In some embodiments, combining the biological sample with a matrix resolving agent or diluent (e.g., comprising the matrix resolving agent) occurs prior to a lysis step, an amplification step, or both. In some embodiments, one or more steps of a method described herein is achieved using a device (e.g., a rapid test device) described herein.

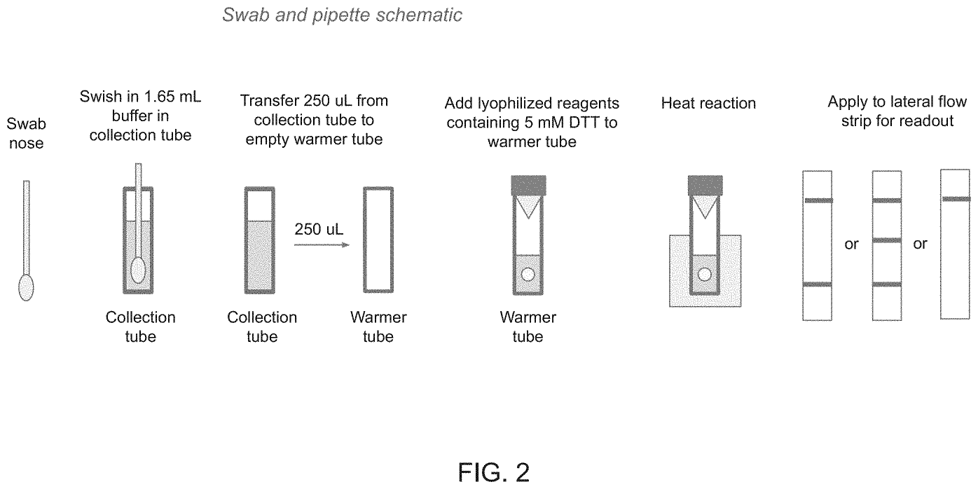

FIG. 2 shows a schematic of an exemplary device and exemplary method for applying, in part, the discovery of the present disclosure. A sample-collecting element, e.g., a swab, may be used to collect a biological sample comprising a mucous matrix containing sample, e.g., a nasal secretion sample. The biological sample may be deposited into a chamber of a device or a reaction tube comprising diluent comprising a matrix resolving agent to create sample-containing fluid. Without wishing to be bound by theory, such a step in an exemplary method and/or device may resolve the mucous matrix of the biological sample, freeing analytes (e.g., nucleic acids) to participate in downstream steps. The sample-containing fluid may be transferred to another chamber of the device or another tube to be subjected to one or more downstream steps, e.g., heating, lysis, amplification, or contact with a lateral flow assay strip.

### Instructions & Software

In some embodiments, a diagnostic system comprises instructions for using a diagnostic device and/or otherwise performing a diagnostic test method. The instructions may include instructions for the use, assembly, and/or storage of the diagnostic device and any other components associated with the diagnostic system. The instructions may be provided in any form recognizable by one of ordinary skill in the art as a suitable vehicle for containing such instructions. For example, the instructions may be written or published, verbal, audible (e.g., telephonic), digital, optical, visual (e.g., videotape, DVD, etc.) or electronic communications (including Internet or web-based communications).

In some embodiments, the instructions are provided as part of a software-based application. In certain cases, the application can be downloaded to a smartphone or device, and then guides a user through steps to use the diagnostic device. In some embodiments, the instructions instruct a user when to add certain reagents and how to do so. For example, in certain instances, the instructions may instruct a user when to add a matrix resolving agent or diluent (e.g., comprising a matrix resolving agent) to a biological sample, and/or how to mix a biological sample with a matrix resolving agent or diluent (e.g., comprising a matrix resolving agent). As a further example, in certain instances, the instructions may instruct a user when to change reaction tube caps and how to release reagents (e.g., a matrix resolving agent) from the reaction tube caps (e.g., by depressing a button, twisting a portion of the reaction tube cap, etc.).

In some embodiments, a software-based application may be connected (e.g., via a wired or wireless connection) to one or more components of a diagnostic system. In some embodiments, a diagnostic systems comprises or is associated with software to read, transmit, communicate, and/or analyze test results. In some embodiments, a device (e.g., a camera, a smartphone) is used to generate an image of a test result (e.g., one or more lines detectable on a lateral flow assay strip).

### Reaction Mixtures

The disclosure is further directed, in part, to a sample preparation mixture comprising one or more reagents that which prepare a biological sample, e.g., for a method or device described herein. In some embodiments, a sample preparation mixture comprises one or more matrix resolving agents (e.g., a matrix resolving agent described herein), and one or more lysis reagents (e.g., a lysis reagent described herein). In some embodiments, the sample preparation mixture further comprises a diluent. In some embodiments, the components of the sample preparation mixture are comprised in separate sealed containers (e.g., as a kit). In some embodiments, the sample preparation mixture is accompanied with instructions for applying the sample preparation mixture to a biological sample (e.g., comprising a mucous matrix).

### EXAMPLES

### Example 1: Comparing Detection Rates Under Various Sample Processing Protocols

Lateral flow immunoassays are simple to use diagnostic assays that can quickly and visibly report the presence or absence of a target analyte (e.g., a virus or pathogen). Thus, they are attractive and commonly used components of affordable point-of-care diagnostic tests. Biological samples obtained from subjects can comprise mucous matrices, which may comprise insoluble components or viscoelastic components which can interfere with lysis of cells, dispersion of target analytes, amplification of nucleic acids, and/or flow of amplified nucleic acid along the lateral flow assay strip. For example, a sample from the nasal cavity, e.g., a nasal secretion, e.g., an anterior nares specimen, can comprise nasal matrix, which can decrease the accessibility of cells to lysis reagents, the dispersion of target nucleic acids from lysed cells, and the amplification of said target nucleic acids.

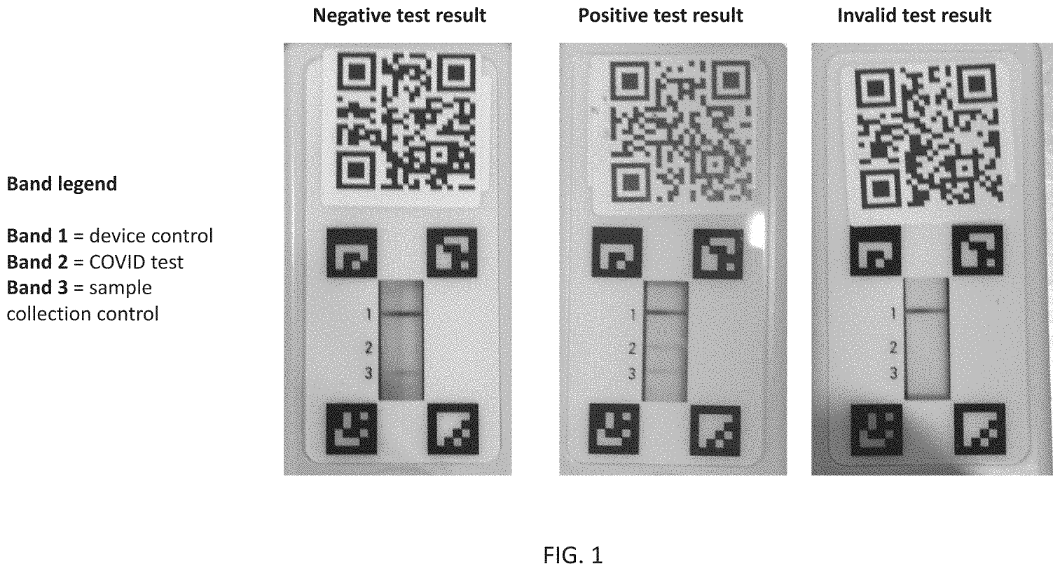

In some aspects, the disclosure relates to a SARS-CoV-2 test where patient material is combined with amplification reagents to amplify a target analyte. The amplification reaction produces amplicons only in the presence of SARS-CoV-2 nucleic acid, as indicated by a visual nucleic acid lateral flow immunoassays NALFIA result (FIG. 1). Although there are advantages to utilizing accessible subject samples like nasal secretions in such a SARS-CoV-2 test, use of a test sample comprising mucous matrices (e.g., nasal matrix) can produce false negative or invalid test results (see, e.g., FIG. 1) despite the presence of SARS-CoV-2 nucleic acid in the test sample. Resolution of the mucous matrix to increase dispersion of cells and target analyte in the subject sample, improve lysis, and/or improve amplification of target analyte would represent a considerable improvement to the accuracy and rate of detection a diagnostic kit for SARS-CoV-2.

This example describes combining nasal secretion samples with a diluent comprising a matrix resolving agent prior to lysis and prior to amplification of the target analyte, and comparing the detection rate of SARS-CoV-2 with samples treated under different protocols. Nasal secretion samples were taken from human subjects. The nasal secretion samples were subjected to different treatments prior to being applied to a method of the disclosure using a device of the disclosure to detect a human control nucleic acid in the sample. Detection rates of the method and device were calculated for each treatment and data were compared to standard operating procedure to obtain p-values (Table 1).

TABLE 1

| | # human | # nasal | Human control | Statistically |
|---|---|---|---|---|
| | control | swab | detection | different from SOP |
| Method | detected | samples | rate (%) | (p-value s 0.05) |
| Standard operating protocol | 65 | 112 | 58% | |
| Elevated temperature | | | | |
| Pre-heat to 75 C., 10 min (prior to bead addition) | 6 | 10 | 60% | 0.260 |
| Pre-heat to 85 C., 10 min (prior to bead addition) | 8 | 10 | 80% | 0.115 |
| Additional Bst polymerase | | | | |
| Twice our SOP amount of Warmstart Bst2.0 | 3 | 9 | 33% | 0.102 |
| Five times our SOP amount of Warmstart Bst2.0 | 5 | 9 | 56% | 0.268 |
| Additional RNase inhibitors | | | | |
| Supplemental RNase inhibitor cocktail | 4 | 12 | 33% | 0.066 |
| Supplemental murine RNase inhibitor to 1 U/μL | 8 | 21 | 38% | 0.047 |
| Supplemental murine RNase inhibitor to 1.5 U/μL | 5 | 8 | 63% | 0.282 |
| PVSA in collection buffer 150 μg/mL | 5 | 9 | 56% | 0.268 |
| PVSA in collection buffer 500 μg/mL | 4 | 9 | 44% | 0.199 |
| PVSA in collection buffer 750 μg/mL | 16 | 20 | 80% | 0.036 |
| PVSA in collection buffer 1 mg/mL | 2 | 4 | 50% | 0.363 |
| Reducing agents | | | | |
| 2 mM DTT added with lyophilized reagents | 11 | 14 | 79% | 0.081 |
| 5 mM DTT added with lyophilized reagents | 78 | 97 | 80% | 0.000 |
| 10 mM DTT added with lyophilized reagents | 4 | 7 | 57% | 0.302 |
| 5 mM TCEP added with lyophilized reagents | 7 | 9 | 78% | 0.153 |
| Rinse and swab transfer (RAST) | | | | |
| Rinse swab in 100 μL, then transfer to sample collection buffer (RAST) | 3 | 5 | 60% | 0.352 |
| Rinse swab in 200 μL, then transfer to sample collection buffer (RAST) | 1 | 4 | 25% | 0.181 |
| Rinse swab in 330 μL, then transfer to sample collection buffer (RAST) | 23 | 34 | 68% | 0.098 |
| Rinse swab in 400 μL, then transfer to sample collection buffer (RAST) | 4 | 4 | 100% | 0.121 |
| Rinse swab in 500 μL, then transfer to sample collection buffer (RAST) | 7 | 12 | 58% | 0.240 |
| Rinse swab in 600 μL, then transfer to sample collection buffer (RAST) | 2 | 4 | 50% | 0.363 |
| Rinse swab in 800 μL, then transfer to sample collection buffer (RAST) | 3 | 4 | 75% | 0.336 |
| Swab and Pipette transfer (SAP) | | | | |
| Swab into 330 μL, use pipet to transfer to make 2X dilution | 3 | 6 | 50% | 0.298 |
| Swab into 330 μL, use pipet to transfer to make 3X dilution | 32 | 35 | 91% | 0.000 |
| Swab into 330 μL, use pipet to transfer to make 4X dilution | 21 | 24 | 88% | 0.004 |
| Swab into 330 μL, use pipet to transfer to make 5X dilution | 46 | 57 | 81% | 0.002 |
| Swab into 330 μL, use pipet to transfer to make 10X dilution | 29 | 32 | 91% | 0.000 |
| Swab into 330 μL, use pipet to transfer to make 50X dilution | 26 | 32 | 81% | 0.009 |
| Swab into 330 μL, use pipet to transfer to make 100X dilution | 9 | 10 | 90% | 0.038 |
| Swab and Pipette transfer + liquid additives (SAP+) | | | | |
| Swab into 330 μL, use pipet to transfer to make 2X dilution + 5 mM DTT | 28 | 28 | 100% | 0.000 |
| Swab into 330 μL, use pipet to transfer to make 3X dilution + 5 mM DTT | 27 | 27 | 100% | 0.000 |
| Swab into 330 μL, use pipet to transfer to make 5X dilution + 5 mM DTT | 105 | 108 | 97% | 0.000 |
| Swab into 1.65 mL, transfer 250 μL + 1 mM DTT | 10 | 12 | 83% | 0.061 |
| Swab into 1.65 mL, transfer 250 μL + 2 mM DTT | 10 | 12 | 83% | 0.061 |
| Swab into 1.65 mL, transfer 250 μL + 5 mM DTT | 107 | 109 | 98% | 0.000 |
| Swab into 330 μL, use pipet to transfer to make 2X dilution + 750 μg/ | 7 | 10 | 70% | 0.209 |
| mL PVSA | | | | |
| Swab into 330 μL, use pipet to transfer to make 5X dilution + 750 μg/ | 7 | 10 | 70% | 0.209 |
| mL PVSA | | | | |
| Swab and Pipette transfer + lyophilized additives in beads (SAP+) | | | | |
| Swab into 1.65 mL, transfer 250 μL + 5 mM lyophilized DTT in | 37 | 39 | 95% | 0.000 |
| lyophilized bead | | | | |
| Swab into 1.65 mL, transfer 250 μL + 10 mM lyophilized DTT in | 37 | 39 | 95% | 0.000 |
| lyophilized bead | | | | |
| Alternative swabbing mechanisms | | | | |
| Clear nose with rayon swab first, then use Detect swab | 19 | 28 | 68% | 0.112 |
| Reduced flocking “O2” swab + 5 mM DTT | 24 | 29 | 83% | 0.008 |
| Additional strategies | | | | |
| PVSA in collection buffer (750 μg/mL) + 5 mM DTT | 5 | 8 | 63% | 0.282 |
| PVSA in collection buffer (750 μg/mL) + 10 mM DTT | 6 | 8 | 75% | 0.200 |
| 200 μM EGTA in collection buffer + 5 mM DTT | 8 | 10 | 80% | 0.115 |
| Swab into 330 μl, use pipet to transfer to make 2X dilution + 5 mM DTT + 1 | 11 | 14 | 79% | 0.081 |
| U/ul superase-in | | | | |

The data in Table 4 show that treating a nasal secretion sample with an exemplary matrix resolving agent, DTT, significantly increased the detection rate of the method and the device. The matrix resolving agent could be provided in solid (e.g., lyophilized) form. In addition, the data show that treating the nasal secretion sample with a diluent significantly increased the detection rate of the method and the device. Dilution as great as 100× showed significant improvement in detection rate. Further, the data showed that treating the sample with both an exemplary matrix resolving agent (DTT) and a diluent provided the most significant improvement to detection rate observed. Such improvements in detection rate may allow more accurate detection of target nucleic acids in subject samples containing mucous matrices, enabling more accurate, point-of-care diagnoses of diseases like SARS-CoV-2.

### Example 2: Evaluating Forms of Exemplary Matrix Resolving Agent DTT

This example examines whether there is a difference in the effectiveness of DTT, an exemplary matrix resolving agent examined in Example 1, based upon whether the DTT is provided in solid, lyophilized form or added aqueously to a sample to be processed.

Biological samples were obtained and treated as in Example 1 (Table 1), and DTT was either prepared lyophilized with one or more lysis reagents or added to the rehydrated one or more lysis reagents prior to treatment of the sample. The data show no significant difference in the detection rate obtained between lyophilizing the DTT or adding the DTT to rehydrated reagents (Tables 2 and 3).

TABLE 2

| | | | | Human |
|---|---|---|---|---|
| | | # human | # nasal | control |
| Reducing | | control | swab | detection |
| agent | Date | detected | samples | rate (%) |
| DTT added | 11-Sep | 19 | 20 | 95% |
| fresh to | 11-Sep | 10 | 10 | 100% |
| rehydrated | 13-Sep | 9 | 10 | 90% |
| lyophilized | 14-Sep | 20 | 20 | 100% |
| reagents | 18-Sep | 12 | 12 | 100% |
| | 10-Sep | 9 | 9 | 100% |
| | 11-Sep | 18 | 19 | 95% |
| | 14-Sep | 16 | 17 | 94% |
| | 14-Sep | 2 | 3 | 67% |
| | 4-Sep | 29 | 30 | 97% |
| | 7-Sep | 10 | 10 | 100% |
| | 7-Sep | 5 | 5 | 100% |
| | 8-Sep | 18 | 18 | 100% |
| | SUM | 177 | 183 | 97% |
| DTT | 15-Sep | 8 | 8 | 100% |
| lyophilized | 15-Sep | 21 | 22 | 95% |
| along with | 17-Sep | 8 | 9 | 89% |
| reagents | 22-Oct | 19 | 20 | 95% |
| | SUM | 56 | 59 | 95% |

TABLE 3

| | | Not | |
|---|---|---|---|
| | Detected | detected | |
| Fresh reducing agent | 177 | 6 | 183 |
| lyophilized reducing agent | 56 | 3 | 59 |
| | 233 | 9 | |

This example demonstrates that lyophilized or freshly prepared DTT may be used effectively as a matrix resolving agent in the methods or devices of the disclosure.

### Example 3: Evaluating Chelators as Matrix Resolving Agents

Metal ions are potent inhibitors of LAMP. This Example demonstrates that addition of chelators to diluent (e.g., to collection buffer) relieves inhibition of methods of detecting target nucleic acids by metal ions. Exemplary chelator EGTA was investigated at 200 uM and 500 uM as a matrix resolving agent in qLAMP of target nucleic acids from nasal matrix-containing samples. qLAMP (quantitative Loop-mediated Isothermal Amplification) is a method whereby amplification is tracked through real-time fluorescence monitoring that indicates the presence and/or accumulation of amplified nucleic acid products.

Four subjects swabbed two nasal samples each into 2 separate tubes containing either (1) 3% Tween, 1.5% Tween, 200 μM EGTA, or 500 μM EGTA or (2) no matrix resolving additive and then qLAMP was performed to detect endogenous RNase P. Each sample was split into 3 technical replicates. EGTA and Tween were added to buffer prior to insertion of swabs. Beads, primers and Evagreen were added after addition of nasal samples to buffer.

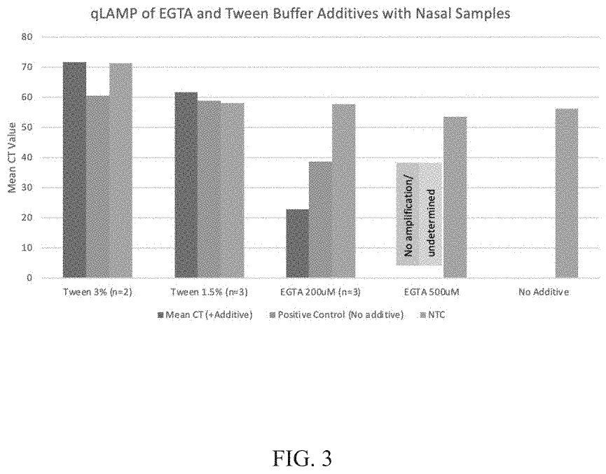

Results showed that 200 μM EGTA decreased mean CT values relative to Tween or controls (FIG. 3, and reduced CT value by 41% relative to the same subject controls.

200 μM EGTA was combined with reducing agent (5 mM DTT) and target nucleic acid detection rate was assessed using 5c/μL Accuplex (a synthetic SARS-CoV-2 virus sample comprising synthetic RNA surrounded by viral capsid sold by Seracare) as an exemplary target nucleic acid in LAMP detected by LFA strip. Use of 200 μM EGTA resulted in 100% RNase P (RP) target nucleic acid detection and exemplary SARS-CoV-2 target nucleic acid (Accuplex) detection on LFA strip readout with nasal matrix. The results showed that EGTA addition to sample-containing fluids improved the detection of exemplary target nucleic acids.

TABLE 4

| | RP Detection Rate | CoV Detection Rate |
|---|---|---|
| 5 mM DTT + 200 μM EGTA | 9/9 (100%) | 9/9 (100%) |
| 5 mM DTT-EGTA | 8/9 (88.9%) | 7/9 (77.8%) |

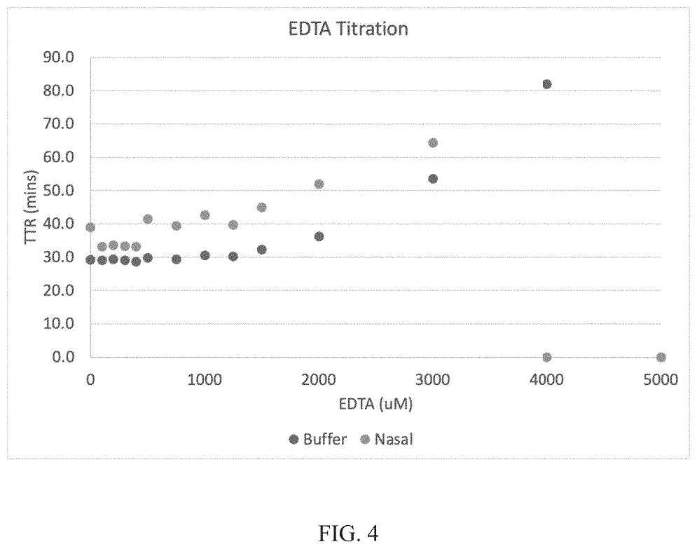

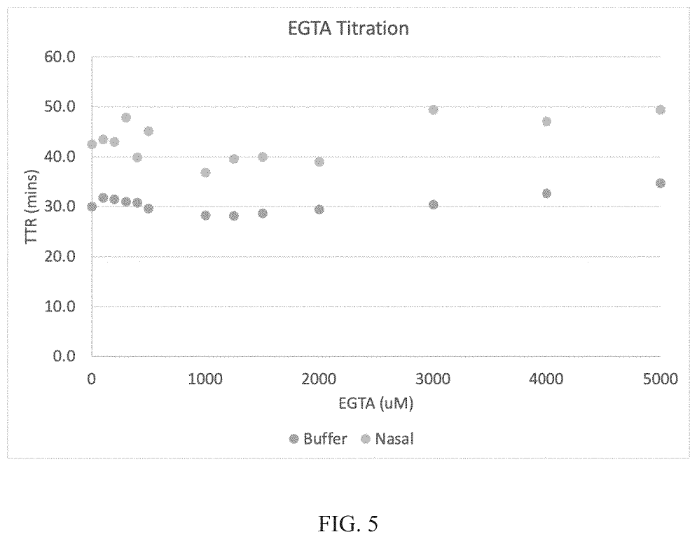

Titration curves measuring time to result (TTR) for EDTA or EGTA in samples containing nasal matrix were produced to evaluate the exemplary chelator's effects on qLAMP. 1250 copies of SARS-CoV-2 nucleic acid target were used in qLAMP reactions. A range of EDTA and EGTA concentrations from 0-5000 μM were tested. EDTA appeared most effective at reducing TTR in nasal samples between 100-500 μM (FIG. 4). EGTA appeared most effective at 1000 μM (FIG. 5). Data are averages from multiple qLAMP experiments with multiple patient samples. However, EDTA has a negative impact on SARS-CoV-2 TTR in a dose dependent manner (data not shown) whereas EGTA does not. The results showed that EGTA may be most effective at 1000 μM in matrix containing samples.

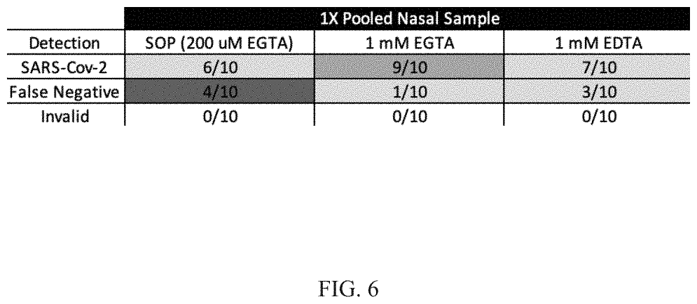

A study was conducted to evaluate EDTA and EGTA effects on detection of SARS-CoV-2 target nucleic acids from frozen swab nasal matrix containing samples. 200 μM EGTA, 1000 μM EGTA, and 1000 μM EDTA were evaluated. 1× pooled nasal samples were made with 2,500 genomic copies/reaction of heat-inactivated SARS-CoV-2 virus input. SARS-CoV-2 target nucleic acids were detected by LAMP LFA. 1000 μM EGTA reactions had improved true positive and false positive rates relative to 200 μM EGTA reactions and 1000 μM EDTA reactions (FIG. 6). The results showed that EGTA at 1000 μM improves detection of a target nucleic acid relative to other concentrations of EGTA and other chelators.

A limit of detection (LOD) experiment was performed to evaluate the effect of 1000 μM EGTA on detection of SARS-CoV-2 target nucleic acid in 1× nasal matrix containing samples using LAMP LFA.

TABLE 5

| | 1 mM EGTA | | |
|---|---|---|---|
| | 4785 Copies/ | 2500 Copies/ | 625 Copies/ |
| Detection | Reaction | Reaction | Reaction |
| SARS-Cov-2 | 20/20 | 20/20 | 7/10 |
| False Negative | 0/20 | 0/20 | 3/10 |
| Invalid | 0/20 | 0/20 | 0/10 |

The results showed that reactions containing 1000 μM EGTA accurately detected SARS-CoV-2 target nucleic acid. In addition, 200 μM EGTA at 625 copies/reaction resulted in 3/10 detection (data not shown). These data show that 1000 μM EGTA is superior to 200 μM EGTA with regard to limit of detection of SARS-CoV-2 target nucleic acid in 1× nasal matrix.

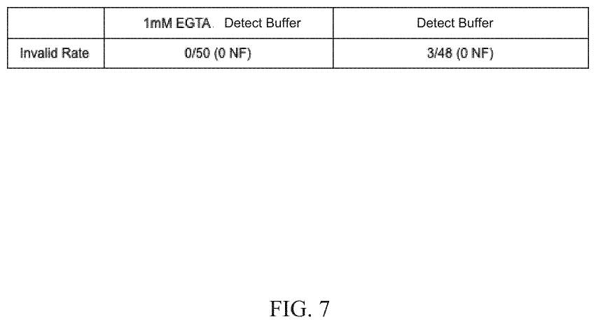

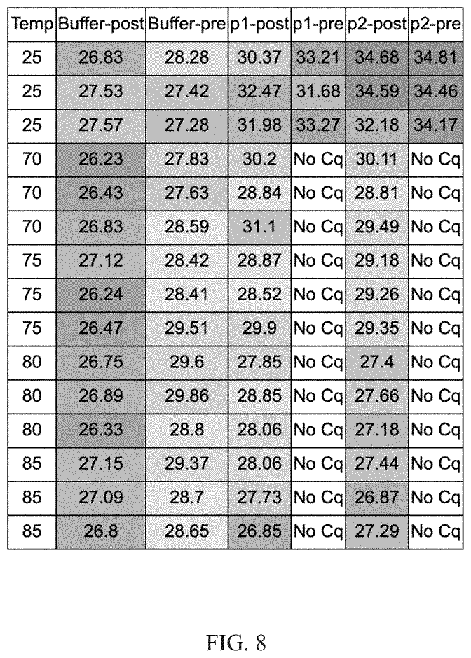

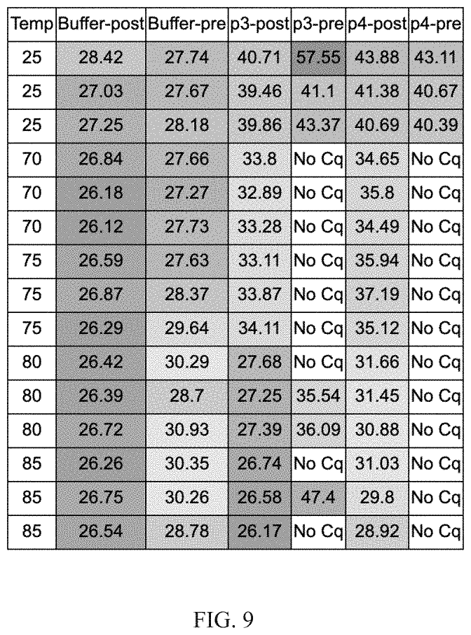

A study was performed to compare the invalid sample rate of LAMP LFA assays detecting SARS-CoV-2 target nucleic acid spiked in nasal matrix containing samples having 1000 μM EGTA vs. 200 μM EGTA (FIG. 7). The results show that 1000 μM EGTA is as good or superior to 200 μM EGTA with respect to invalidity rate in detecting an exemplary target nucleic acid.

The preceding data in this Example suggested that an increase in EGTA, e.g., to 1000 μM, could allow for a shorter incubation time of a biological sample or sample-containing fluid containing a mucous matrix in a method to detect a target nucleic acid (e.g., a SARS-CoV-2 target nucleic acid). A study was performed in duplicate to compare the invalidity rate in a LAMP LFA assay against SARS-CoV-2 target nucleic acid using 200 μM vs 1000 μM EGTA both at a 30 minute incubation time. After both studies it was found that 200 μM EGTA had a 7.9% invalid rate vs 1000 μM EGTA at 3.4% invalid rate with a 30 minute incubation period. These results showed that increasing EGTA to 1000 μM can enable a significant reduction in assay incubation time while keeping the invalid rate below 5%.

### Study 1:

200 μM EGTA: 4/52 (7.7%) Invalid (4 RP neg, 0 No flow)
1000 μM EGTA: 1/52 (1.9%) Invalid (1 RP neg, 0 No flow)
Note: “RP neg.” refers to an invalid result when the RNase P human control gene is not detected, while “No flow” refers to an invalid result when the sample fails to flow down the lateral flow strip.

### Study 2:

200 μM EGTA: 3/37 (8.1%) Invalid (3 RP neg, 0 No flow)
1000 μM EGTA: 2/37 (5.4%) Invalid (2 RP neg, 0 No flow)
Overall from Both Studies:
200 μM EGTA: 7/89 (7.9%) Invalid (7 RP neg, 0 No flow)
1000 μM EGTA: 3/89 (3.4%) Invalid (3 RP neg, 0 No flow)

### Example 4: Evaluating Pre-Heating Samples to Resolve Matrices

Studies were conducted to determine whether pre-heating biological samples or sample-containing fluids could improve the time to determination (e.g., to a negative or positive result) for a quantitative loop mediated isothermal amplification test (qLAMP) in mucous matrix containing samples. Here, “pre” heating refers to heating a sample matrix prior to the addition of one or more reagents required for RT-LAMP and initiation of nucleic acid amplification.

Biological samples obtained from real subjects (e.g., patients) may contain both a sample matrix and, if “positive”, a nucleic acid target of interest (e.g., from an infectious pathogen). In this Example, the ability to separate sample matrix from target analyte in a laboratory setting was leveraged to create samples by adding sample matrix and exemplary nucleic acid target separately, allowing examination of the role of either component in the effects of pre-heating on target nucleic acid detection. Studies were conducted to investigate whether amplification of an exemplary nucleic acid target from a sample matrix resolved by treatment with heat depended on whether that nucleic acid target was present in the sample matrix prior to heating or added subsequently once the matrix had already been heated.

Nasal swabs were sourced from four separate donors, “p1,” “p2,” “p3” or “p4.” Nasal swabs were eluted in a lysis/amplification reagent and the matrix was either (1) mixed with heat inactivated SARS-CoV-2 virus and subsequently heated at a designated temperature (“p #-pre”) OR (2) heated at the designated temperature, followed by the addition of heat inactivated SARS-CoV-2 virus (“p #-post”). In a control condition, the lysis/amplification reagent alone was heated as a mixture with the nucleic acid target (“buffer-pre”) or heated separately prior to the addition of heat-inactivated SARS-COV-2 nucleic acid (“buffer-post”). This sample preparation step of heating and mixing with the nucleic acid target was followed in all cases by the subsequent addition of RT-LAMP reagents and measurement of SARs-CoV-2 amplification time (in minutes) by qLAMP. The results showed that heating a sample matrix and then adding a nucleic acid target to the resolved matrix material decreased the time to reach detected levels of the amplified target nucleic acid material. In contrast, when a sample containing both the sample matrix and the exemplary nucleic acid target were heated together prior to amplification, no amplification was detected. This suggested that the timing of pre-heating is important to obtaining the benefits of applying heat and suggested that a component of the lysis and/or amplification reagents may interfere with amplification and/or accelerate the degradation of a nucleic acid target only when it is present during heating but not if the nucleic acid is added after heating.

Studies were performed to determine which specific component in the lysis/amplification buffer interfered with the stability and/or amplification of the nucleic acid target when that nucleic acid was heated together with a sample matrix as a contrived positive sample in advance of preparing a nucleic acid amplification reaction.

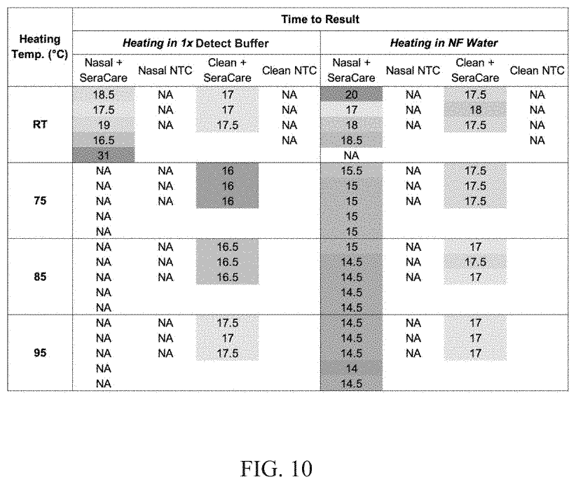

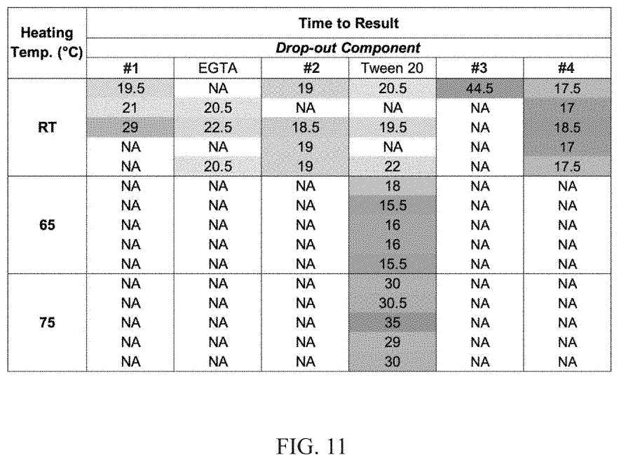

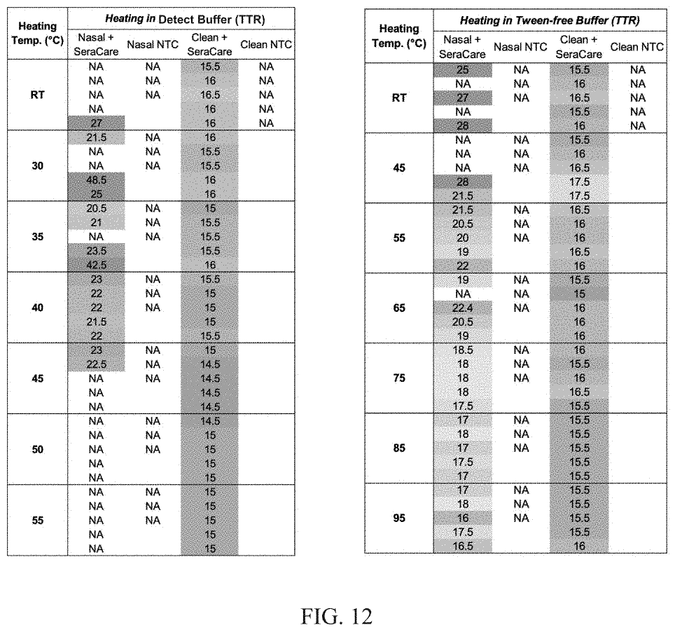

In this experiment, a human sample was eluted into a lysis/amplification buffer, a target nucleic acid template was added to the eluted sample and then the mixture was heated at the designated temperature for 5 minutes followed by the addition of RT-LAMP reagents and qLAMP amplification. qLAMP to detect SeraCare synthetic encapsulated SARS-CoV-2 virus target nucleic acid (or no template control (NTC)) mixed with or without nasal matrix were performed either preheating to various temperatures in the presence of a lysis/amplification buffer or in nuclease-free water (NF water) (FIG. 10). The results showed that heating a sample containing an encapsulated exemplary nucleic acid target in water shows improved speed and sensitivity of amplification of a target nucleic acid relative to pre-heating in the lysis/amplification buffer. A further experiment was performed excluding individual buffer components (FIG. 11) from the experiment conducted in FIG. 10 (buffer components 1, 2, 3, 4, EGTA, or Tween 20). In this experiment, a human sample was eluted into a lysis/amplification buffer, a target nucleic acid template was added to the eluted sample and then the mixture was heated at the designated temperature for 5 minutes followed by the addition of RT-LAMP reagents and qLAMP amplification. The lysis/amplification buffer lacked the indicated component (“Drop out”). The results showed that pre-heating a sample matrix containing a nucleic acid target of interest in the presence of Tween 20 interfered with subsequent amplification of that nucleic acid target and that excluding Tween 20 or the buffer containing it (e.g., by pre-heating in water) improves amplification. Without wishing to be bound by theory, it is possible that Tween affects the stability and/or integrity of the encapsulated nucleic acid target such that it is degraded during the heating process. Further experiments were performed to examine additional temperatures and determine whether pre-heating with Tween-free buffer (omitting just the Tween from the lysis/amplification buffer) could improve amplification (FIG. 12). The results showed that pre-heating as low as 65° C. could improve the speed and sensitivity of amplification.

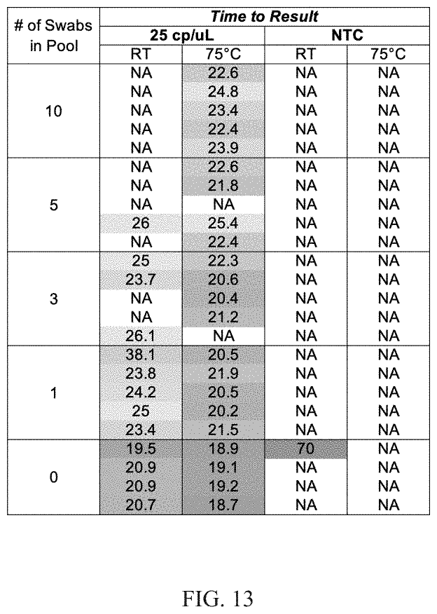

The COVID-19 pandemic has stressed organizations and health systems, including their ability to rapidly screen a large number of biological samples accurately and sensitively. One common practice to more efficiently screen large numbers of samples is sample pooling, however pooling can exacerbate the interfering effects of mucous matrices that might be present in the samples. To evaluate whether pre-heating might improve the speed and sensitivity of amplification of a target nucleic acid in pooled samples, various numbers of nasal matrix containing swab samples were pooled, spiked with Seracare synthetic SARS-COV-2 virus target nucleic acid (or no template for the no template control (NTC)) and either preheated to 75° C. or kept at room temperature (RT), and the time to reach detectable levels of amplified target nucleic acid was measured using qLAMP LFA (FIG. 13). The results showed that pre-heating enabled amplification of target nucleic acid in pools of 10 nasal matrix containing samples or 5, and improved time to detectable levels of amplified target nucleic acid in pools of 3 samples. These results suggested that pre-heating may improve, and indeed enable, sample pooling in methods of target nucleic acid detection described herein.

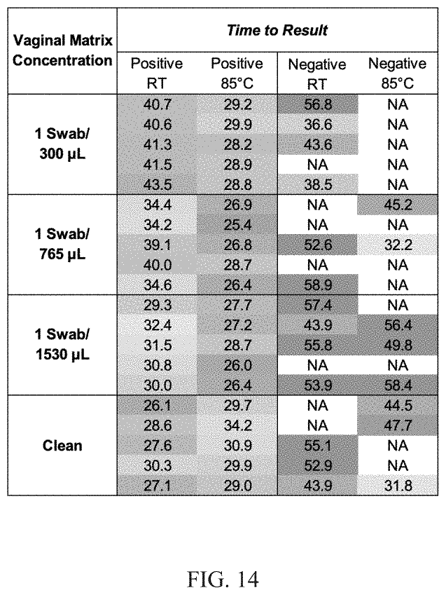

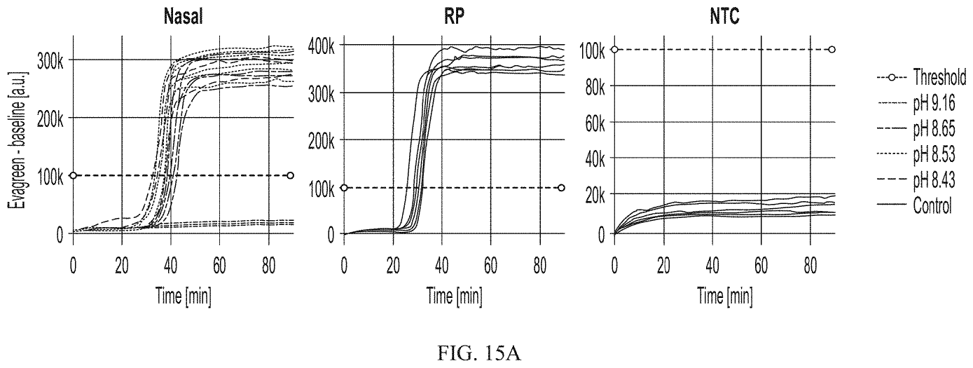

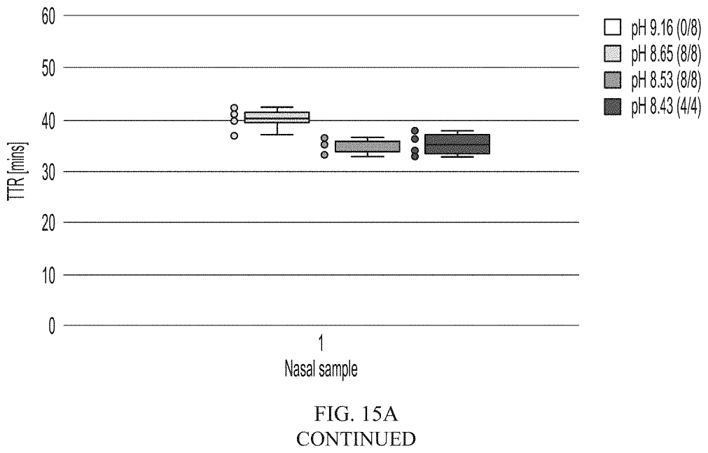

Experiments were conducted to determine whether pre-heating would improve the speed or sensitivity of amplification of a target nucleic acid in samples comprising vaginal matrices. A vaginal matrix sample was dispersed in varying diluent volumes, with or without spiking with Seracare synthetic viral target nucleic acid (positive) or non-target control nucleic acid (negative) and either preheated to 85° C. or not (RT) and the time to reach detectable levels of amplified target nucleic acid was measured through qLAMP (FIG. 14). The results showed that pre-heating can improve the speed and sensitivity of amplification of samples containing vaginal matrices, particularly in samples dispersed in smaller volumes (300 μL and 765 μL), suggesting that the benefits of matrix resolution, e.g., by pre-heating, extend to any matrix containing sample.

### Example 5: Evaluating the Role of Diluent pH in Resolving Matrices

Experiments were conducted to determine the effect of sample-containing fluid pH on time to result (TTR) of qLAMP of target nucleic acid in the presence of an exemplary mucous matrix, nasal matrix.

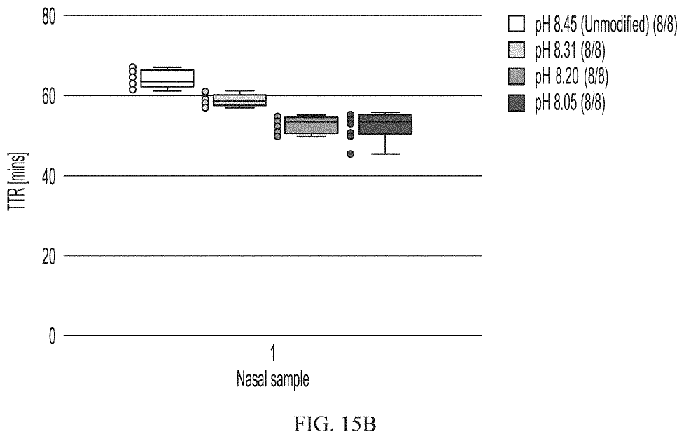

In one experiment, the pH of a pooled nasal sample was evaluated and then modified sample-containing fluids were produced at different pH. Unmodified nasal sample with amplification reagents (enzymes and primers) had a pH of 8.35 in this experiment and the buffer was measured at 8.58. Four different modified sample-containing fluid pHs were selected: 9.16, 8.65, 8.53, and 8.43. Samples were run with 8 technical replicates on qLAMP for 90 minutes, or 4 technical replicates for pH 8.43. The TTR of qLAMP at each pH was determined (FIG. 15A). At pH 9.16, no amplification was detected (data not shown). Samples at pHs 8.65, 8.53, and 8.43 exhibited similar TTR of qLAMP to unadjusted control sample. Adjusting the pH to 8.65 produced a slower TTR of qLAMP than pHs 8.53 or 8.43. These results suggest that decreasing the pH of a sample in the range tested improves the efficiency of amplification and detection of a target nucleic acid.

In a further experiment, samples were prepared by taking frozen swabs and creating a large 2× nasal matrix (double the standard concentration) containing master solution. To this 2× mix, a 2× reaction mix was added and pH measured. The sample was then aliquoted and pH adjusted to the indicated value. Samples were run with 8 technical replicates on qLAMP for 90 minutes (FIG. 15B). The results showed that, over the range of pH tested (8.05-8.45), adjusting the sample pH to 8.20 or 8.05 improved the speed of amplification of a target nucleic acid in samples comprising nasal matrices.

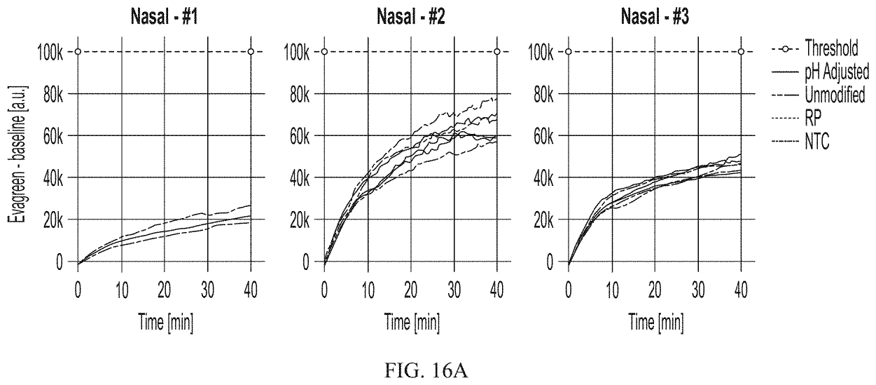

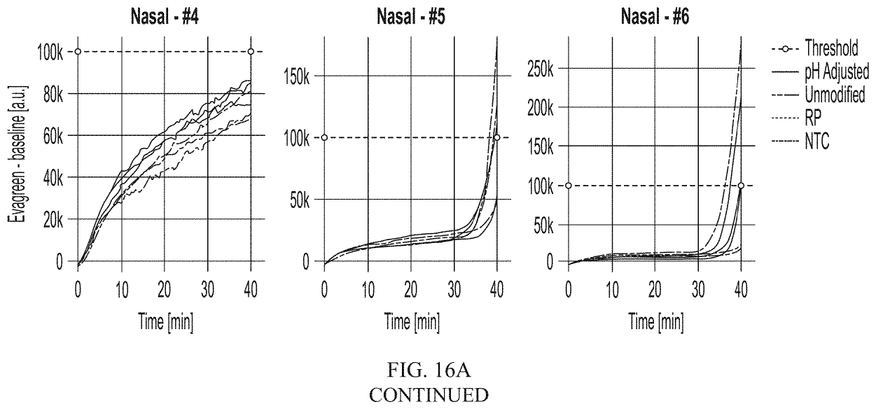

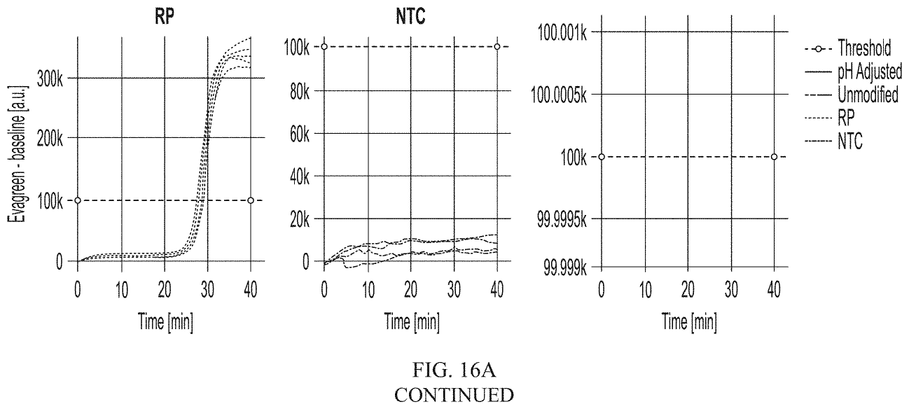

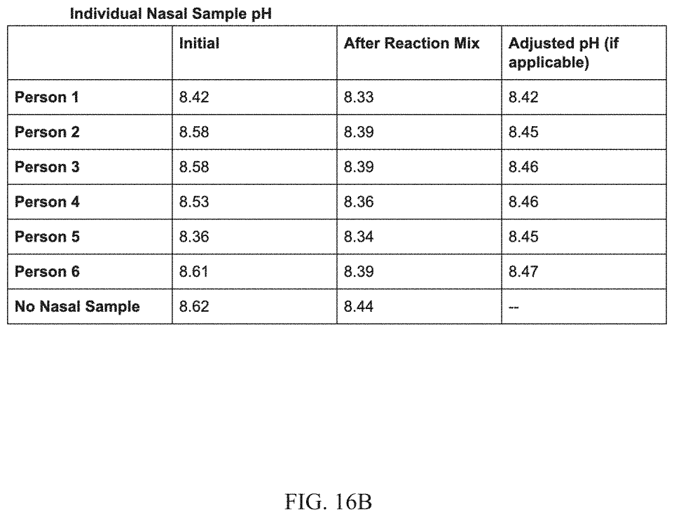

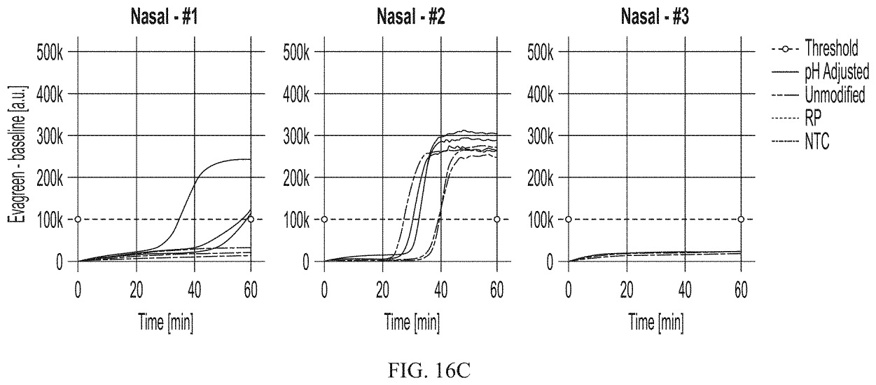

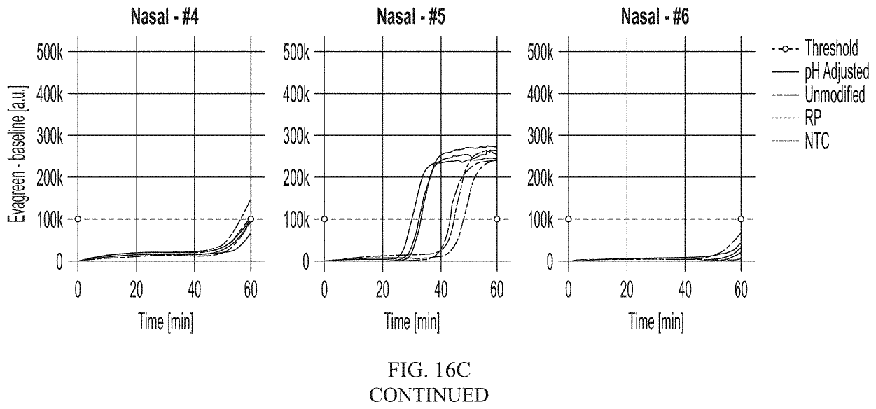

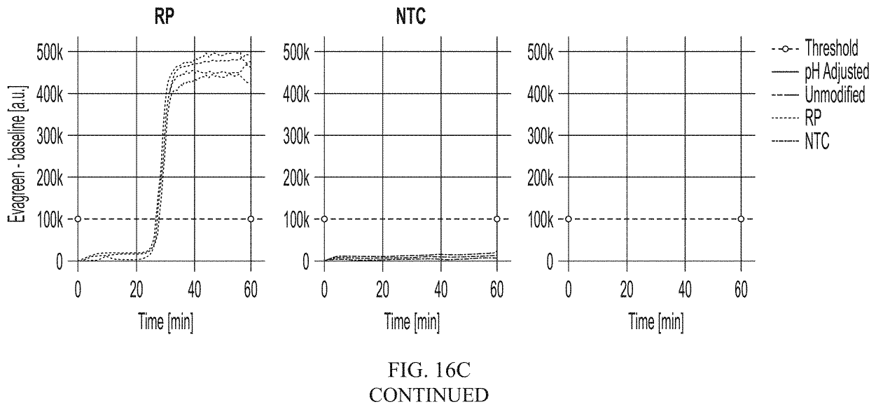

In a further experiment, six individual nasal matrix-containing samples were evaluated for pH before and after having added 2× reaction mix (FIG. 16B). The results showed that the final sample pH was primarily dependent on the pH of the reaction mix. For example, one nasal sample had an initial pH of 8.58 and another had an initial pH of 8.36, and when the reaction mix was added to each of these samples the pH of the two converged somewhat to 8.39 and 8.34, respectively. This suggests that while different noses can produce nasal matrix-containing samples of differing pH, the addition of a buffer can help to normalize resulting sample-containing fluids for these differences. Each individual's sample was split into two aliquots: one aliquot had its pH adjusted to the pH of collection buffer and reaction mix, and the other aliquot for each person was left unmodified. qLAMP was performed on the adjusted and unmodified aliquots (FIG. 16A). The results showed that samples 1-4 did not produce an amplification reaction while samples 5 and 6 did. In reactions that did occur, there was no difference in TTR between unadjusted and adjusted samples. The results for qLAMP reactions where amplification occurred showed that the small adjustment in pH between unadjusted and adjusted aliquots did not produce a change in amplification efficiency

Additional experiments were performed to evaluate the effects of adjusting sample-containing fluid pH to 8. qLAMP was performed to detect a target nucleic acid in 6 nasal matrix containing samples after adjusting the sample-containing fluid pH to 8 by addition of diluent at suitable pH or leaving the sample-containing fluid unmodified by adding a diluent that did not modify pH. qLAMP reactions were run for 60 minutes with monitoring of sample fluorescence (Evagreen fluorescence) (FIG. 16C). The results showed no amplification in 3/6 samples; pH modification increased the speed at which amplification generated detectable product in 2/6 samples; and pH modification rescued the lack of amplification in 1/6 samples. Overall the results from the 6 samples showed that adjusting the pH of a matrix containing sample, e.g., by controlling the pH of the diluent added to the sample (e.g., to produce a pH of about 8), can improve detection of amplification of a target nucleic acid, e.g., the speed of amplification of a target nucleic acid in samples comprising nasal matrices.

Overall, the results showed that as pH decreases from about 9 to about 8, the detection of amplification (e.g., the efficiency of RT-LAMP) in the presence of nasal matrix improves. The data shows that pH of biological samples varied significantly from sample to sample, and that in general lower pH (e.g., a pH about 8) in a sample-containing fluid is as good or better than higher pH in regards to detection of amplification in nasal matrix-containing samples.

### Example 6: Evaluating RNase Inhibitors in Matrix Resolution

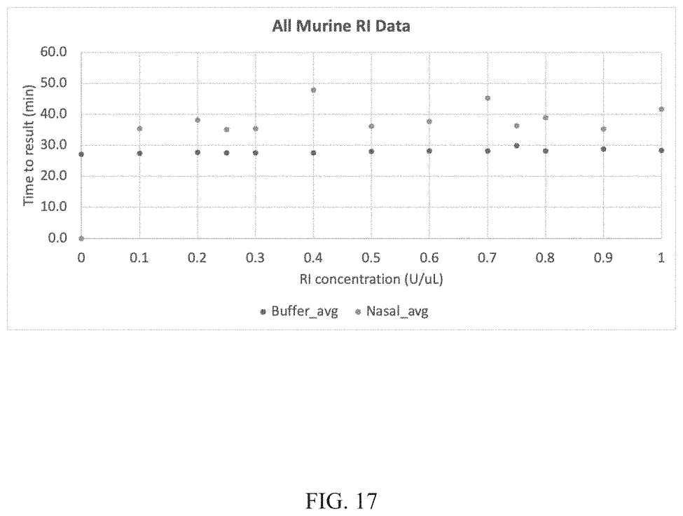

Methods and compositions of the disclosure can be used to detect a target nucleic acid comprising RNA. Target RNAs may be vulnerable to RNase enzymes present in samples, e.g., in mucous matrix containing samples. However, RNase inhibitors can be costly and so inclusion of excess inhibitor is not desirable. Experiments were performed to determine whether including an exemplary murine RNase inhibitor in qLAMP samples containing nasal matrices and SARS-CoV-2 target nucleic acid (synthetic SARS-CoV-2 RNA Full Genome (supplied by Twist BioScience)) can improve detection of the target nucleic acid. Promega RNasin Plus was used as an exemplary murine RNase inhibitor. RNase inhibitor was added to sample-containing fluid, qLAMP reactions were run, and time to result (TTR) was measured and averaged across 4 different nasal matrix containing samples at varying concentrations of RNase inhibitor (FIG. 17). The results showed that all samples lacking RNase inhibitor failed to detect the target RNA, whereas all concentrations of RNase inhibitor tested (as low as 0.1 U/μL) were sufficient to enable detection of target RNA, showing that RNase inhibitor improves detection of the target nucleic acid.

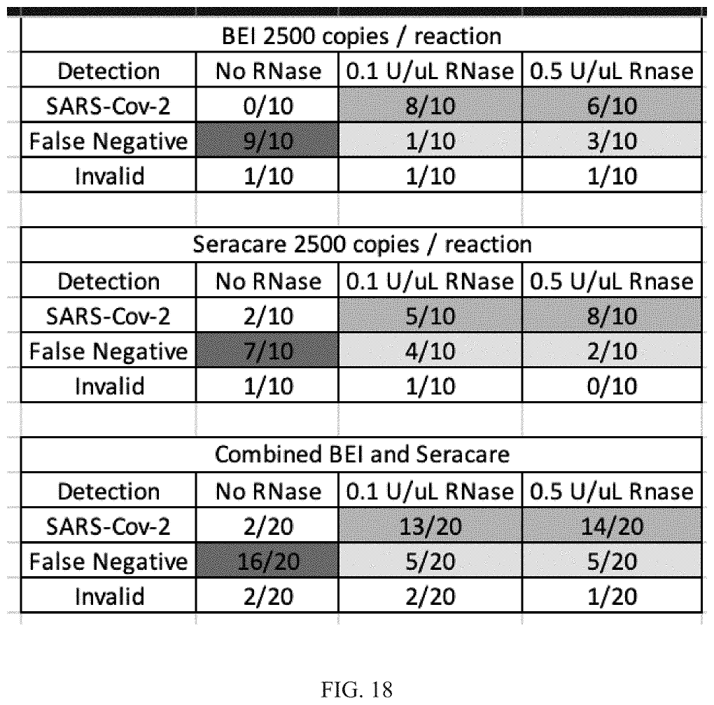

LAMP LFA experiments were performed on 10 different nasal matrix containing samples spiked with either heat-inactivated SARS-CoV-2 virus (BEI) or Seracare synthetic encapsulated SARS-CoV-2 virus and including RNase inhibitor at two different concentrations (FIG. 18). The results showed that detection of SARS-CoV-2 target nucleic acid was significantly lower in samples that did not include RNase inhibitor and that there was little meaningful difference between 0.1 U/μL and 0.5 U/μL of RNase inhibitor.

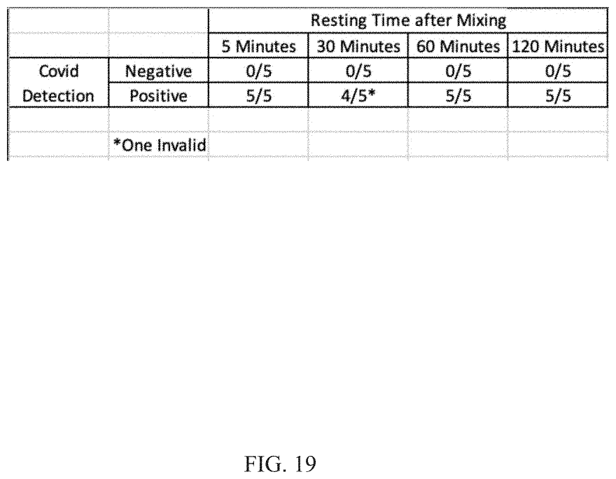

Pooled nasal samples were used to determine if 0.1 U/μL RNase inhibitor was sufficient to allow various room temperature rest periods for sample-containing fluids without degradation of the target nucleic acids (FIG. 19). The results showed that target nucleic acid was detected in almost all samples (1 invalid time point at 30 minutes), demonstrating that 0.1 U/μL RNase inhibitor sufficiently inhibits any RNase present in nasal matrix samples to enable effective detection of a target nucleic acid, even with rest times of up to two hours.

Together these results show that detection of target nucleic acid is improved by inclusion of an RNase inhibitor in samples containing a mucous matrix.

### Example 7: Blood Matrices and Urine Matrices

A series of experiments were performed to demonstrate the applicability of methods and compositions of the disclosure to samples containing blood matrices or urine matrices. Blood and urine samples are both attractive options for assays detecting target analytes, e.g., target nucleic acids, particularly those which are inaccessible or have low availability in samples from orifices such as nose, mouth, and ears.

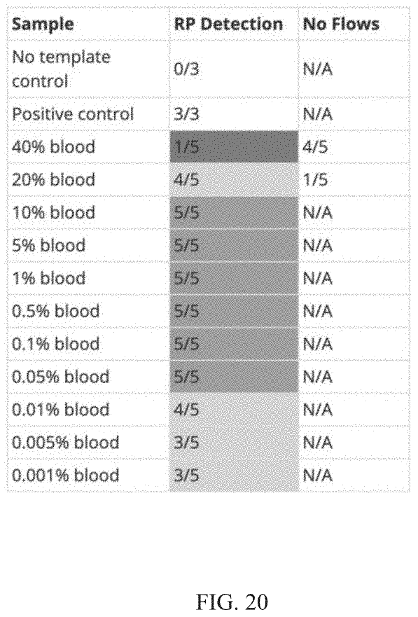

To determine both the extent of dilution necessary (e.g., to dilute blood mucous matrices sufficiently) for amplification and detection of a target nucleic acid and the dilution limit of detection, a blood sample was diluted to varying degrees (40%, 20%, 10%, 5%, 1%, 0.5%, 0.1%, 0.05%, 0.01%, 0,005%, or 0.001% v/v), separated into aliquots, and LAMP was conducted for a target nucleic acid encoding RNase P (RP). Amplified nucleic acid was detected using lateral flow assay (LFA) (FIG. 20). The results showed that flow issues at 20% and 40% dilutions prevented LFA detection of 1/5 and 4/5 samples, respectively, but that the remaining samples detected RP. 10% dilution aliquots all detected RP successfully without flow problems, demonstrating that biological samples containing blood matrices can be analyzed at up to at least 10% v/v dilution. Dilution down to 0.05% detected RP in all aliquots tested, with 4/5 RP positive aliquots at 0.01% and 3/5 aliquots at each of 0.005% and 0.001% dilutions. These results showed that exemplary target nucleic acid encoding RP can be detected in blood with high reliability down to 0.05% dilution and detected with some reliability down at least 0.001% dilution. The results showed that for methods of detecting a target nucleic acid in a blood sample containing blood matrices and comprising contacting an LFA strip, a 10% dilution or greater enables consistent accurate detection of a target nucleic acid.

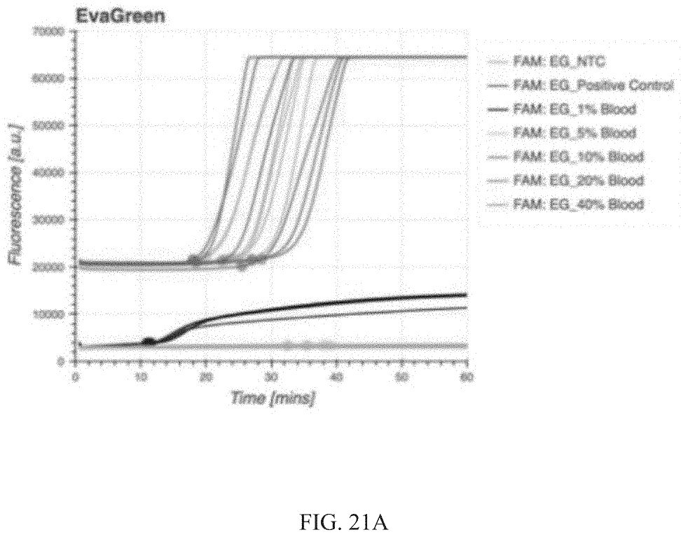

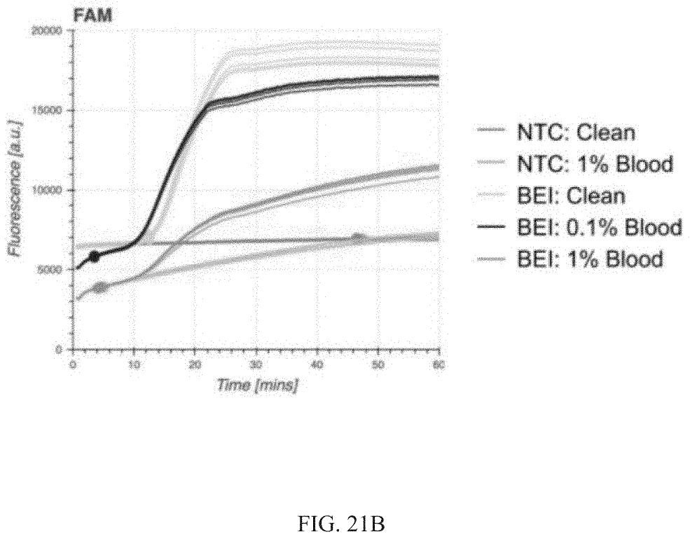

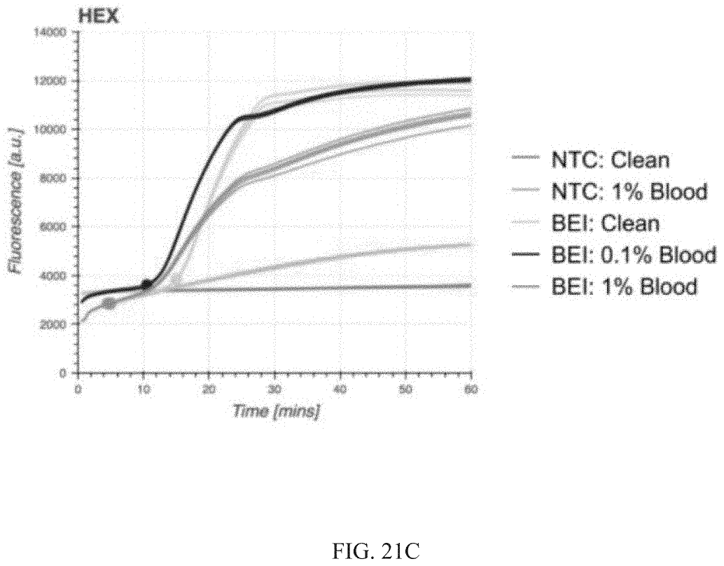

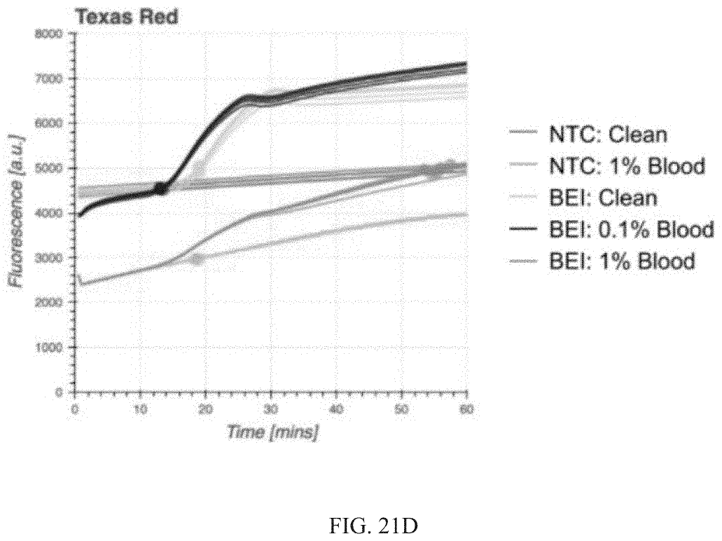

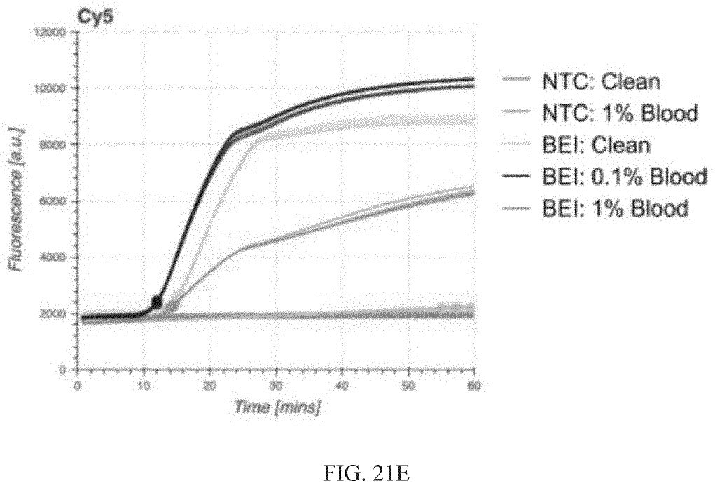

Additional experiments were conducted to detect RP target nucleic acid in a blood sample using qLAMP. EvaGreen intercalating fluorescent dye (FIG. 21A) was used to detect qLAMP amplification RP target nucleic acid at a variety of dilutions of a blood sample (40%, 20%, 10%, 5%, and 1%). Amplification was only detected at 1% dilution. Without wishing to be bound by theory, blood matrices may inhibit fluorescence and/or fluorescence detection of dyes relied upon by quantitative real-time amplification monitoring techniques at all but significant dilution levels. The results showed, however, that amplification of a target nucleic acid using a blood sample containing blood matrices can be successfully achieved at a 1% dilution or greater. A series of fluorophore-conjugated probes (FAM: FIG. 21B; HEX: FIG. 21C; Texas Red: FIG. 21D; Cy5: FIG. 21E) were used to detect amplification of heat-inactivated SARS-CoV-2 virus input (BEI) target nucleic acid mixed with blood sample blood matrices at 1% and 0.1% dilutions. The results showed that 0.1% and 1% dilutions enabled detection of SARS-CoV-2 target nucleic acids from heat-inactivated samples using fluorescence-based quantitative real-time amplification monitoring techniques such as qLAMP.

To determine the extent of dilution necessary (e.g., to dilute urine mucous matrices sufficiently) for amplification and detection of a target nucleic acid, 6 different urine samples (3 female, 2 male) were diluted to varying degrees, separated into aliquots, and LAMP was conducted for a target nucleic acid encoding RNase P (RP) (endogenous or spiked) or spiked SARS-COV-2 amplicon (either RNA or DNA). Amplified nucleic acid was detected using a lateral flow assay (LFA) and data for the least dilution to detect RP is provided in the table below. The results showed that RP was detected in 4/6 samples at 20% dilution and 2/6 samples at 10%, spiked SARS-CoV-2 RNA amplicon was detected in 2/6 samples, and that spiked SARS-CoV-2 DNA amplicon was detected in 4/6 samples. The results showed that exemplary target nucleic acid encoding RP or SARS-CoV-2 spike can be detected in urine at 10% or 20% dilution. The results showed that for methods of detecting a target nucleic acid in a urine sample containing urine matrices and comprising contacting an LFA strip, a 10% dilution or greater enables detection of a target nucleic acid.

TABLE 6

| | | pH | Tolerance | | | Spiked | Spiked | Covid |
|---|---|---|---|---|---|---|---|---|
| | | of 20% | in the | | RP | Covid | Covid | DNA |
| | | urine in | LAMP | Endogenous | detected | RNA | DNA | LOD |
| Urine | pH of | Detect | reaction | RP | when | amplicon | amplicon | (copies/ |
| pool | urine | buffer | (% v/v) | detected? | spiked? | detected? | detected? | reaction) |
| Female 1 | 6.45 | 7.24 | 20 | 5/5 | 5/5 (2,940 | 0/5 (294,000 | No (8,232 | N/A |
| | | | | | copies/ | copies/ | copies/ | |
| | | | | | reaction) | reaction) | reaction) | |
| Female 2 | 5.92 | 6.91 | 10 | 1/3 | 3/3 (14,700 | 3/3 (5,880 | Yes | 4,116 |
| | | | | | copies/ | copies/ | | |
| | | | | | reaction) | reaction) | | |
| Female 3 | 5.74 | 7.44 | 20 | 3/3 | 2/3 (14,700 | 0/3 (5,880 | Yes | 4,116 |
| | | | | | copies/ | copies/ | | |
| | | | | | reaction) | reaction) | | |
| Male 1 | 7.68 | 7.69 | 20 | 2/3) | 2/3 (14,700 | 0/3 (5,880 | Yes | 8,232 |
| | | | | | copies/ | copies/ | | |
| | | | | | reaction) | reaction) | | |
| Male 2 | 8.41 | 7.98 | 10 | 2/3 | 3/3 (14,700 | 0/3 (5,880 | No (8,232 | 8,232 |
| | | | | | copies/ | copies/ | copies/ | |
| | | | | | reaction) | reaction) | reaction) | |
| Male 3 | 6.78 | 7.83 | 20 | 2/3 | 3/3 (at | 3/3 (5,880 | Yes | 2,499 |
| | | | | | 14,700 | copies/ | | |
| | | | | | copies/ | reaction) | | |
| | | | | | reaction) | | | |

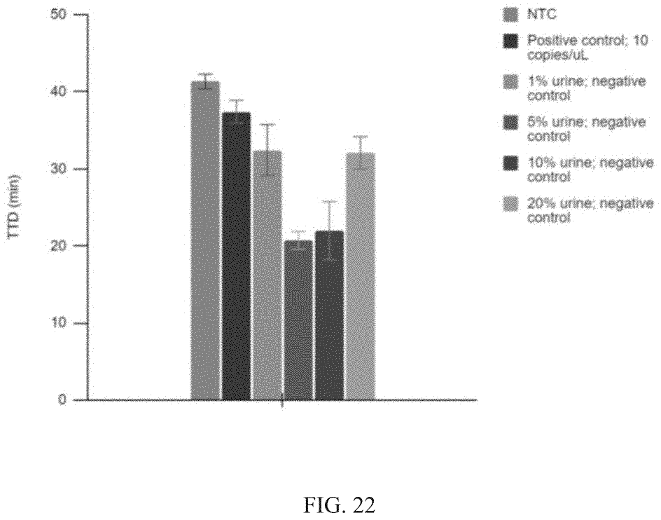

Additional experiments were conducted to detect endogenous RP target nucleic acid in a urine sample using qLAMP. EvaGreen intercalating fluorescent dye (FIG. 22) was used to detect qLAMP amplification of RP target nucleic acid at a variety of dilutions of a urine sample (40%, 20%, 10%, 5%, and 1%). NTC indicates a no urine sample and no template (RP target nucleic acid) control; Positive control indicates added template (added RP target nucleic acid); Negative control indicates no added template (i.e., detection of endogenous RP target nucleic acid). Amplification was not detected at 40% dilution (data not shown). Amplification was detected at 20%, 10%, 5%, and 1% dilutions, with the lowest time to determination (TTD) seen for 5% and 10% dilutions. The results showed that amplification of a target nucleic acid using a urine sample containing urine matrices can be successfully achieved at a 20% dilution or greater. The results further showed that 5% and 10% dilutions provided improved speed of detection of an exemplary target nucleic acid using fluorescence-based quantitative real-time amplification monitoring techniques such as qLAMP.

1. A method for detecting a target nucleic acid, the method comprising:
combining a biological sample with a diluent and/or at least one matrix resolving agent to produce a sample-containing fluid; and
contacting a lateral flow assay strip having a first end and a second end with the sample-containing fluid, the lateral flow assay strip comprising:
an absorbent substrate having a first end and a second end; and
an indicator region arranged on the substrate and configured to indicate the presence of the target nucleic acid.

2. The method of claim 1, further comprising allowing the sample-containing fluid to move from the first end of the lateral flow assay strip to the indicator region.

3. A method for detecting a target nucleic acid, the method comprising:
combining a biological sample with a diluent and/or at least one matrix resolving agent to produce a sample-containing fluid; and
contacting a nucleic acid detection device with the sample-containing fluid and using the nucleic acid detection device to detect the target nucleic acid.

4. A rapid test system configured to detect a target nucleic acid, the system comprising:
a housing;
a diluent and/or at least one matrix resolving agent;
at least one lysis reagent;
at least one amplification reagent; and
either:
a lateral flow assay strip accommodated in the housing and arranged to receive an amplified sample, the lateral flow assay strip comprising:
an absorbent substrate having a first end and a second end, and
an indicator region arranged on the substrate and configured to indicate the presence or absence of the target nucleic acid by interaction with the amplified sample; or
a nucleic acid detection device.

5. The method of claim 1, wherein the diluent comprises the matrix resolving agent.

6-7. (canceled)

8. The method of claim 1, wherein the biological sample comprises a mucous matrix.

9. The method of claim 8, wherein the mucous matrix is a nasal matrix.

10. The method of claim 1, wherein the biological sample is an anterior nares specimen and/or comprises a nasal secretion.

11. The method of claim 1, wherein the at least one matrix resolving agent comprises a reducing agent.

12. The method of claim 11, wherein the reducing agent is selected from the group consisting of DTT (dithiothreitol), glutathione, DTE (dithioerythritol), TCEP, 2-mercaptoethanol, and any combination thereof.

13. The method of claim 12, wherein the reducing agent is DTT.

14. The method of claim 1, wherein the at least one matrix resolving agent comprises a mucolytic agent.

15. The method of claim 1, wherein the at least one matrix resolving agent comprises one or more enzymes.

16. The method of claim 15, wherein the one or more enzymes comprises a metalloproteinase, a disintegrin and metalloproteinase with thromospondin motifs (ADAMTS) family protein, a functional fragment or variant of any thereof, an RNase inhibitor, and/or a protease.

17-18. (canceled)

19. The method of claim 1, wherein the at least one matrix resolving agent comprises a chelator.

20. The method of claim 19, wherein the chelator comprises EGTA.

21. The method of claim 1, wherein the diluent has a pH of about 8 or 8.1.

22. The method of claim 1, wherein the diluent further comprises one or more lysis reagents, and/or wherein the method further comprises combining the biological sample or sample-containing fluid with one or more lysis reagents.

23. (canceled)

24. The method of claim 22, wherein the one or more lysis reagents comprise:
an enzyme chosen from lysozyme, lysostaphin, zymolase, cellulase, protease, glycanase, or any combination thereof; and/or
a detergent comprising sodium dodecyl sulphate (SDS), Tween (e.g., Tween 20 or 80), 3-[(3-cholamidopropyl)dimethylammonio]-1-propanesulfonate (CHAPS), 3-[(3-cholamidopropyl)dimethylammonio]-2-hydroxy-1-propanesulfonate (CHAPSO), Triton X-100, and/or NP-40.

25-28. (canceled)

29. The method of claim 1, comprising applying heat to the biological sample prior to addition of diluent, matrix resolving agent, or other reagent to the biological sample.

30-33. (canceled)
